(19) United States
(12) Patent Application Publication
(10) Pub. No.: US 2025/0074985 A1
(43) Pub. Date: Mar. 6, 2025

(54) STABLE FORMULATIONS OF ANTI-CTLA4 ANTIBODIES ALONE AND IN COMBINATION WITH PROGRAMMED DEATH RECEPTOR 1 (PD-1) ANTIBODIES AND METHODS OF USE THEREOF

(71) Applicant: Merck Sharp & Dohme LLC, Rahway, NJ (US)

(72) Inventors: Soumendu Bhattacharya, East Windsor, NJ (US); Chakravarthy Nachu Narasimhan, Scotch Plains, NJ (US); Manoj K. Sharma, Littleton, MA (US); Xiaoyu Yang, Basking Ridge, NJ (US); Arnab De, Burlington, MA (US); Rubi Burlage, Florham Park, NJ (US); Jason K. Cheung, Westfield, NJ (US)

(73) Assignee: Merck Sharp & Dohme LLC, Rahway, NJ (US)

(21) Appl. No.: 18/882,177

(22) Filed: Sep. 11, 2024

Related U.S. Application Data
(63) Continuation of application No. 16/609,671, filed on Oct. 30, 2019.
(60) Provisional application No. 62/500,268, filed on May 2, 2017.

(51) Int. Cl. C07K 16/28; A61K 9/00; A61K 9/19; A61K 39/00; A61K 47/18; A61K 47/26

(52) U.S. Cl. CPC C07K 16/2818; A61K 47/183; A61K 47/26; A61K 9/0019; A61K 9/19; A61K 2039/507; C07K 2317/565

(57) ABSTRACT

The invention relates to stable formulations comprising antibodies or antigen binding fragments thereof that bind to cytotoxic T lymphocyte associated antigen 4 (CTLA4), optionally further containing an anti-human programmed death receptor 1 (PD-1) antibody or antigen binding fragment thereof. Also provided are methods of treating various cancers and chronic infections with the formulations of the invention.

### CROSS-REFERENCE TO RELATED APPLICATIONS

This application is a continuation of U.S. patent application Ser. No. 16/609,671 filed Oct. 30, 2019, as a 371 National Stage Entry of International Patent Application No. PCT/US2018/030420 filed May 1, 2018, which claims the benefit of U.S. Provisional Patent Application No. 62/500,268, filed May 2, 2017, the entire contents of which are herein incorporated by reference.

### REFERENCE TO SEQUENCE LISTING SUBMITTED ELECTRONICALLY

The contents of the electronic sequence listing (24449-US-CNT-SEQLIST-11SEPT2024.xml; Size: 147,456 bytes; and Date of Creation: Sep. 10, 2024) are herein incorporated by reference in their entirety.

### FIELD OF THE INVENTION

The invention relates to formulations of therapeutic antibodies and their use in treating various disorders. In one aspect, the invention relates to formulations comprising antibodies or antigen binding fragments thereof that bind to cytotoxic T lymphocyte associated antigen 4 (CTLA4). In another aspect, such formulation further comprises an anti-human programmed death receptor 1 (PD-1) antibody or antigen binding fragment thereof. Also provided are methods of treating various cancers and chronic infections with the formulations described herein.

### BACKGROUND OF THE INVENTION

Antibody drugs for use in humans may differ somewhat in the amino acid sequence of their constant domains, or in their framework sequences within the variable domains, but they typically differ most dramatically in the CDR sequences. Even antibodies binding to the same protein, the same polypeptide, or even potentially the same epitope may comprise entirely different CDR sequences. Therapeutic antibodies for use in human beings can also be obtained from human germline antibody sequence or from non-human (e.g., rodent) germline antibody sequences, such as in humanized antibodies, leading to yet further diversity in potential CDR sequences. These sequence differences result in different stabilities in solution and different responsiveness to solution parameters. In addition, small changes in the arrangement of amino acids or changes in one or a few amino acid residues can result in dramatically different antibody stability and susceptibility to sequence-specific degradation pathways. As a consequence, it is not possible at present to predict the solution conditions necessary to optimize antibody stability. Each antibody must be studied individually to determine the optimum solution formulation. Bhambhani et al. (2012) J. Pharm. Sci. 101:1120.

Antibodies are also relatively high molecular weight proteins (˜150,000 Da), for example as compared with other therapeutic proteins such as hormones and cytokines. As a consequence, it is frequently necessary to dose with relatively high weight amounts of antibody drugs to achieve the desired molar concentrations of drug. In addition, it is often desirable to administer antibody drugs subcutaneously, as this enables self-administration. Self-administration avoids the time and expense associated with visits to a medical facility for administration, e.g., intravenously. Subcutaneous delivery is limited by the volume of solution that can be practically delivered at an injection site in a single injection, which is generally about 1 to 1.5 ml. Subcutaneous self-administration is typically accomplished using a pre-filled syringe or autoinjector filled with a liquid solution formulation of the drug, rather than a lyophilized form, to avoid the need for the patient to re-suspend the drug prior to injection. Antibody drugs must be stable during storage to ensure efficacy and consistent dosing, so it is critical that whatever formulation is chosen supports desirable properties, such as high concentration, clarity and acceptable viscosity, and that also maintains these properties and drug efficacy over an acceptably long shelf-life under typical storage conditions.

CTLA4 has very close relationship with the CD28 molecule in gene structure, chromosome location, sequence homology and gene expression. Both of them are receptors for the co-stimulative molecule B7, mainly expressed on the surface of activated T cells. After binding to B7, CTLA4 can inhibit the activation of mouse and human T cells, playing a negative regulating role in the activation of T cells.

CTLA4 mAbs or CTLA4 ligands can prevent CTLA4 from binding to its native ligands, thereby blocking the transduction of the T cell negative regulating signal by CTLA4 and enhancing the responsiveness of T cells to various antigens. In this aspect, results from in vivo and in vitro studies are substantially in concert. At present, there are some CTLA4 mAbs being tested in clinical trials for treating prostate cancer, bladder cancer, colorectal cancer, cancer of gastrointestinal tract, liver cancer, malignant melanoma, etc. (Grosso et al., CTLA-4 blockade in tumor models: an overview of preclinical and translational research. Cancer Immun. 13:5 (2013)).

As important factors affecting the function of T cells, CTLA4 and CTLA4 mAbs can produce specific therapeutic effect on diseases by interfering with the immune microenvironment in the body. They have high efficacy and remedy the deficiency of traditional medication, opening a novel pathway of gene therapy. CTLA4 and CTLA4 mAbs are being tested in experiments and various stages of clinical trials. For example, in autoimmune diseases, they effectively inhibited airway hyperresponsiveness in an animal model of asthma, prevented the development of rheumatic diseases, mediated immune tolerance to an allograft in the body, and the like. On the other hand, although biological gene therapy has not shown any adverse effect in short term clinical trials, attention should be paid to the potential effect after long term application. For example, excessive blockade of CTLA4-B7 signaling by CTLA4 mAbs may result in the development of autoimmune diseases. As antibodies can specifically bind to their antigens and induce the lysis of target cells or block the progress of pathology, development and utilization of drugs based on antibodies, especially humanized antibodies have important significance in the clinical treatment of malignant tumors and other immune diseases in humans.

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245). It has been proposed that the efficacy of anti-PD-1 antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. One such agent that has been tested in combination with antagonists of PD-1 is cytotoxic T lymphocyte associated antigen 4 (abbreviated CTLA4).

The need exists for stable formulations of anti-CTLA4 antibodies for pharmaceutical use, e.g., for treating various cancers and infectious diseases, as well as for stable formulations of anti-CTLA4 antibodies co-formulated with anti-human PD-1 antibodies. Preferably, such formulations will exhibit a long shelf-life, be stable when stored and transported, and will preferably exhibit stability over months to years under conditions typical for storage of drugs for self-administration, i.e., at refrigerator temperature in a syringe, resulting in a long shelf-life for the corresponding drug product.

### SUMMARY OF THE INVENTION

In one aspect, the invention includes a formulation of an anti-CTLA4 antibody, or antigen binding fragment thereof, comprising (i) an anti-CTLA4 antibody, or antigen binding fragment thereof; (ii) a buffer, (iii) a non-reducing sugar; (iv) a non-ionic surfactant; and an antioxidant. In another embodiment, the formulation further comprises an anti-PD-1 antibody, e.g., pembrolizumab or nivolumab. In one embodiment, the formulation further comprises a chelator.

In an embodiment, the formulation comprises (i) about 10 mg/ml to about 200 mg/ml of an anti-CTLA4 antibody, or antigen binding fragment thereof; (ii) about 5 mM to about 20 mM buffer; (iii) about 6% to about 8% weight/volume (w/v) non-reducing sugar; (iv) about 0.01% to about 0.10% non-ionic surfactant; and (v) about 1 mM to about 20 mM anti-oxidant. In another embodiment, the formulation further comprises an anti-PD-1 antibody, e.g., pembrolizumab or nivolumab. In another embodiment, the formulation further comprises a chelator. In one embodiment the chelator is present in amount of about 1 μM to about 50 μM. chelator is DTPA. In one embodiment, the formulation has a pH between 4.5-6.5. In particular embodiments, the pH of the formulation is from about pH 5.0 to about pH 6.0. In a further embodiment, the pH of the formulation is from about pH 5.3 to about pH 5.8. In another embodiment, the pH is 5.3. In another embodiment, the pH is 5.4. In one embodiment, the pH is 5.5. In one embodiment, the pH is 5.6. In a further embodiment, the pH is 5.7. In an embodiment, the pH is 5.8.

In one embodiment of the formulation, the buffer is L-histidine buffer or sodium acetate buffer, the non-reducing sugar is sucrose, the non-ionic surfactant is polysorbate 80, and the anti-oxidant is methionine, or a pharmaceutically acceptable salt thereof. In one embodiment, the anti-oxidant is L-methionine. In another embodiment, the anti-oxidant is a pharmaceutically acceptable salt of L-methionine, such as, for example, methionine HCl.

In another embodiment, formulation comprises (i) about 10 mg/ml to about 200 mg/ml of an anti-CTLA4 antibody, or antigen binding fragment thereof; (ii) about 5 mM to about 20 mM of L-histidine or about 5 mM to about 20 mM of sodium acetate buffer; (iii) about 6% to about 8% w/v sucrose; (iv) about 0.01% to about 0.10% w/v polysorbate 80; and (v) about 1 mM to about 20 mM L-methionine. In another embodiment, the formulation further comprises an anti-PD-1 antibody, e.g., pembrolizumab or nivolumab. In an embodiment, the formulation further comprises a chelator. In one embodiment, the chelator is present in an amount of about 1 μM to about 50 μM. In one embodiment, the chelator is DTPA. In one embodiment the buffer is a L-histidine buffer. In one embodiment, the formulation comprises about 8 mM to about 12 mM of L-histidine. In another embodiment, the formulation comprises about 5 mM to about 10 mM of L-methionine. In a further embodiment, the formulation comprises polysorbate 80 at a weight ratio of approximately 0.02% w/v. In one embodiment, the anti-CTLA4 formulation comprises sucrose at a weight ratio of about 7% (w/v).

In embodiments of the formulation, the concentration of the anti-CTLA4 antibody or antigen binding fragment thereof is from about 10 mg/ml to about 100 mg/ml. In another embodiment, the concentration of the anti-CTLA4 antibody or antigen binding fragment thereof is about 10 mg/ml, 12.5 mg/ml, 15 mg/ml, 20 mg/ml, 25 mg/ml, 50 mg/ml, 75 mg/ml or 100 mg/ml. In one embodiment, the concentration of the anti-CTLA4 antibody or antigen binding fragment thereof is 25 mg/mL. In an additional embodiment, the concentration of the anti-CTLA4 antibody or antigen binding fragment thereof is about 50 mg/ml. In another embodiment, the concentration of the anti-CTLA antibody or antigen binding fragment thereof is about 75 mg/mL. In a further embodiment, the concentration of the anti-CTLA4 antibody or antigen binding fragment thereof is 100 mg/mL.

In one aspect, provided is a formulation comprising about 25 mg/mL of an anti-CTLA4 antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 50 mg/mL of an anti-CTLA4 antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 75 mg/mL of an anti-CTLA4 antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 100 mg/mL of an anti-CTLA4 antibody or antigen binding fragment thereof, 10 mM L-histidine buffer, about 7% w/v sucrose, about 0.02% w/v polysorbate 80, and about 10 mM L-methionine.

In one aspect of any of the above formulations, the formulation has a pH of about 5.3 to 5.8. In another aspect, the formulation has a pH of about 5.5 to about 5.6. In another aspect, the formulation has a pH of about 5.5. In another aspect, the formulation has a pH of about 5.6.

In one aspect of any of the above formulations, the formulation comprises an anti-PD1 antibody or antigen binding fragment thereof. In one aspect the anti-PD1 antibody is pembrolizumab. In another aspect, the anti-PD1 antibody is nivolumab.

In another aspect, the formulation may further comprise a chelator. In one embodiment, the chelator is DTPA. In one embodiment, the chelator is EDTA. In one aspect, the chelator is present in an amount from about 1 μM to about 50 μM. In one embodiment, the formulation comprises about 5 μM of the chelator. In one embodiment, the formulation comprises about 10 UM of the chelator. In one embodiment, the formulation comprises about 15 μM of the chelator. In one embodiment, the formulation comprises about 20 μM of the chelator. In one embodiment, the formulation comprises about 25 μM of the chelator. In one embodiment, the formulation comprises about 30 μM of the chelator. In one embodiment, the formulation comprises about 35 UM of the chelator. In one embodiment, the formulation comprises about 40 μM of the chelator. In one embodiment, the formulation comprises about 45 μM of the chelator. In one embodiment, the formulation comprises about 50 μM of the chelator. In one embodiment, the chelating agent is DTPA, which is present at any of the amounts stated above. In another embodiment, the chelating agent is EDTA which is present at any of the amounts stated above.

In one embodiment, the formulation is contained in a glass vial. In another embodiment, the formulation is contained in an injection device. In another embodiment, the formulation is a liquid formulation. In one aspect, the formulation is frozen to at least below-70° C. In another embodiment, the formulation is a reconstituted solution from a lyophilized formulation.

In certain embodiments, the formulation is stable at refrigerated temperature (2-8° C.) for at least 3 months, preferably 6 months, and more preferably 1 year, and even more preferably up to through 2 years. In one embodiment of the formulation, after 12 months at 5° C. the % monomer of the anti-CTLA4 antibody is ≥90% as determined by size exclusion chromatography. In another embodiment of the formulation, after 12 months at 5° C. the % monomer of the anti-CTLA4 antibody is ≥95% as determined by size exclusion chromatography. In another embodiment of the formulation, after 12 months at 5° C. the % heavy chain and light chain of the anti-CTLA4 antibody is ≥90% as determined by reduced CE-SDS. In another embodiment of the formulation, after 12 months at 5° C. the % heavy chain and light chain of the anti-CTLA4 antibody is ≥95% as determined by reduced CE-SDS. In another embodiment of the formulation, after 12 months at 5° C. the % intact IgG of the anti-CTLA4 antibody is ≥90% as determined by non-reduced CE-SDS. In another embodiment of the formulation, after 12 months at 5° C. the % intact IgG of the anti-CTLA4 antibody is ≥95% as determined by non-reduced CE-SDS.

In one aspect of any of the formulations described above, the formulation comprises an anti-CTLA4 antibody or antigen-binding fragment thereof comprising three light chain CDRs and three heavy chain CDRs, wherein the light chain CDRs comprise CDRL1 of SEQ ID NO: 38, CDRL2 of SEQ ID NO:39, CDRL3 of SEQ ID NO:40 and the heavy chain CDRs comprise CDRH1 of SEQ ID NO: 35, CDRH2 of SEQ ID NO: 36, and CDHR3 of SEQ ID NO: 37. In another aspect, the formulation comprises an anti-CTLA4 antibody or antigen binding fragment thereof comprising a heavy chain variable region comprising SEQ ID NO: 88 and a light chain variable region comprising SEQ ID NO: 48. In another aspect, the formulation comprises an anti-CTLA4 antibody or antigen binding fragment thereof comprising a heavy chain comprising SEQ ID NO: 99 and a light chain comprising SEQ ID NO: 100.

In one aspect, the present invention provides a co-formulation of an anti-CTLA4 antibody, or antigen binding fragment thereof and an anti-human PD-1 antibody, or antigen binding fragment thereof, comprising (i) an anti-CTLA4 antibody, or antigen binding fragment thereof; (ii) an anti-human PD-1 antibody, or antigen binding fragment thereof, (ii) a buffer, (iii) a non-reducing sugar; (iv) a non-ionic surfactant; and an antioxidant. In an embodiment, the co-formulation further comprises a chelator. In one embodiment the chelator is EDTA. In another embodiment, the chelator is DTPA. In one embodiment of the co-formulation, the ratio of the anti-human PD-1 antibody to the anti-CTLA4 antibody is 1:2. In another embodiment of the co-formulation, the ratio of the anti-human PD-1 antibody to the anti-CTLA4 antibody is 1:1. In a further embodiment of the co-formulation, the ratio of the anti-human PD-1 antibody to the anti-CTLA4 antibody is 2:1. In another embodiment of the co-formulation, the ratio of the anti-human PD-1 antibody to the anti-CTLA4 antibody is 10:1. In a further embodiment of the co-formulation, the ratio of the anti-human PD-1 antibody to the anti-CTLA4 antibody is 1:10. In another embodiment, the ratio of the anti-human PD-1 antibody to the anti-CTLA4 antibody is 8:1. In a further embodiment, the ratio of the anti-human PD-1 antibody to the anti-CTLA4 antibody is 8:3.

In an embodiment of the invention, the co-formulation comprises (i) about 1 mg/ml to about 100 mg/ml of an anti-CTLA4 antibody, or antigen binding fragment thereof; (ii) about 1 mg/ml to about 100 mg/ml of an anti-human PD-1 antibody (ii) about 5 mM to about 20 mM buffer; (iii) about 6% to about 8% weight/volume (w/v) non-reducing sugar; (iv) about 0.01% to about 0.10% non-ionic surfactant; and (v) about 1 mM to about 20 mM anti-oxidant. In an embodiment, the co-formulation further comprises a chelator. In one embodiment, the chelator is DTPA. In one embodiment of the co-formulation, the ratio of the anti-human PD-1 antibody to the anti-CTLA4 antibody is 1:2. In another embodiment of the co-formulation, the ratio of the anti-human PD-1 antibody to the anti-CTLA4 antibody is 1:1. In a further embodiment of the co-formulation, the ratio of the anti-human PD-1 antibody to the anti-CTLA4 antibody is 2:1. In another embodiment of the co-formulation, the ratio of the anti-human PD-1 antibody to the anti-CTLA4 antibody is 10:1. In a further embodiment of the co-formulation, the ratio of the anti-human PD-1 antibody to the anti-CTLA4 antibody is 1:10. In another embodiment, the ratio of the anti-human PD-1 antibody to the anti-CTLA4 antibody is 8:1. In a further embodiment, the ratio of the anti-human PD-1 antibody to the anti-CTLA4 antibody is 8:3. In one embodiment, the co-formulation has a pH between 4.5 and 6.5. In other embodiments, the pH of the formulation is from about pH 5.0 to about pH 6.0. In a further embodiment, the pH of the formulation is from about pH 5.3 to about pH 5.8.

In one embodiment of the co-formulation, the buffer is a histidine buffer or sodium acetate buffer, the non-reducing sugar is sucrose, the non-ionic surfactant is polysorbate 80, and the anti-oxidant is methionine or a pharmaceutically acceptable salt thereof. In one embodiment, anti-oxidant is L-methionine. In another embodiment, anti-oxidant is a pharmaceutically acceptable salt of L-methionine, such as, for example, methionine HCl.

In another aspect, the co-formulation comprises (i) about 1 mg/ml to about 100 mg/ml of an anti-CTLA4 antibody, or antigen binding fragment thereof; (ii) about 1 mg/ml to about 100 mg/ml of an anti-human PD-1 antibody or antigen binding fragment thereof; (iii) about 5 mM to about 20 mM of L-histidine buffer or about 5 mM to about 20 mM of sodium acetate buffer; (iv) about 6% to about 8% w/v sucrose; (v) about 0.01% to about 0.10% w/v polysorbate 80; and (vi) about 1 mM to about 20 mM L-methionine. In an embodiment, the co-formulation further comprises a chelator. In one embodiment, the chelator is DTPA. In one embodiment, the buffer is L-histidine buffer. In one embodiment, the co-formulation comprises about 8 mM to about 12 mM of L-histidine buffer. In another embodiment, the co-formulation comprises about 5 mM to about 10 mM of L-methionine. In a further embodiment, the co-formulation comprises polysorbate 80 at a weight ratio of approximately 0.02% w/v. In one embodiment, co-formulation comprises sucrose at a weight ratio of about 7% (w/v).

In embodiments of the co-formulation, the concentration of the anti-CTLA4 antibody or antigen binding fragment thereof is from about 1 mg/mL to about 100 mg/mL. In another embodiment, the concentration of the anti-CTLA4 antibody is from about 10 mg/ml to about 100 mg/ml. In another embodiment, the concentration of the anti-CTLA4 antibody or antigen binding fragment thereof is about 10 mg/ml. In another embodiment, the concentration of the anti-CTLA4 antibody or antigen binding fragment thereof is 1.25 mg/ml. In another embodiment, the concentration of the anti-CTLA4 antibody or antigen binding fragment thereof is 2.5 mg/ml. In another embodiment, the concentration of the anti-CTLA4 antibody or antigen binding fragment thereof is 5 mg/ml. In another embodiment, the concentration of the anti-CTLA4 antibody or antigen binding fragment thereof is 12.5 mg/ml. In a further embodiment, the concentration of the anti-CTLA4 antibody or antigen binding fragment thereof is 25 mg/ml. In a further embodiment, the concentration of the anti-CTLA4 antibody or antigen binding fragment thereof is 50 mg/ml. In another embodiment, the anti-CTLA4 antibody or antigen biding fragment thereof is 75 mg/ml. In another embodiment, the concentration of the anti-CTLA4 antibody or antigen binding fragment thereof is 100 mg/ml. In an additional embodiment, the concentration of the anti-CTLA4 antibody or antigen binding fragment thereof is about 50 mg/ml. In another embodiment, the concentration of the anti-CTLA4 antibody is 2.9 mg/mL. In another embodiment, the concentration of the anti-CTLA4 antibody is 7.9 mg/mL.

In embodiments of the co-formulation, the concentration of the anti-human PD-1 antibody is from about 1 mg/mL to about 100 mg/mL. In another embodiment, the concentration of the anti-human PD-1 antibody is about 10 mg/ml to about 100 mg/ml. In another embodiment, the concentration of the anti-human PD-1 antibody is about 25 mg/ml. In another embodiment, the concentration of the anti-human PD-1 antibody is about 22.7 mg/ml. In another embodiment, the concentration of the anti-human PD-1 antibody is about 2.27 mg/ml. In another embodiment, the concentration of the anti-human PD-1 antibody is about 21.1 mg/ml. In another embodiment, the concentration of the anti-human PD-1 antibody is about 23.5 mg/ml.

In one embodiment, the co-formulation comprises about 25 mg/mL of the anti-PD1 antibody, about 12.5 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 25 mg/mL of the anti-PD1 antibody, about 25 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 25 mg/mL of the anti-PD1 antibody, about 50 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 22.72 mg/mL of the anti-PD1 antibody, about 2.3 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 2.27 mg/mL of the anti-PD1 antibody, about 22.7 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 23.5 mg/mL of the anti-PD1 antibody, about 2.9 mg/mL of the anti-CTLA4 antibody, 10 mM L-histidine buffer, about 7% w/v sucrose, about 0.02% w/v polysorbate 80, and about 10 mM L-methionine.

In one embodiment, the co-formulation comprises about 21.1 mg/mL of the anti-PD1 antibody, about 7.9 mg/mL of the anti-CTLA4 antibody, 10 mM L-histidine buffer, about 7% w/v sucrose, about 0.02% w/v polysorbate 80, and about 10 mM L-methionine.

In one aspect of any of the formulations described above, the formulation comprises an anti-CTLA4 antibody or antigen-binding fragment thereof comprising three light chain CDRs and three heavy chain CDRs, wherein the light chain CDRs comprise CDRL1 of SEQ ID NO: 38, CDRL2 of SEQ ID NO:39, CDRL3 of SEQ ID NO:40 and the heavy chain CDRs comprise CDRH1 of SEQ ID NO: 35, CDRH2 of SEQ ID NO: 36, and CDHR3 of SEQ ID NO: 37. In another aspect, the formulation comprises an anti-CTLA4 antibody or antigen binding fragment thereof comprising a heavy chain variable region comprising SEQ ID NO: 88 and a light chain variable region comprising SEQ ID NO: 48. In another aspect, the formulation comprises an anti-CTLA4 antibody or antigen binding fragment thereof comprising a heavy chain comprising SEQ ID NO: 99 and a light chain comprising SEQ ID NO: 100. In one aspect of any of the formulations described above, the anti-human PD-1 antibody or antigen binding fragment thereof comprises three light chain CDRs and three heavy chain CDRs, wherein the light chain CDRs comprise CDRL1 of SEQ ID NO: 1, CDRL2 of SEQ ID NO:2, CDRL3 of SEQ ID NO:3 and the heavy chain CDRs comprise CDRH1 of SEQ ID NO: 6, CDRH2 of SEQ ID NO: 7, and CDHR3 of SEQ ID NO: 8. In another aspect, the formulations comprise an anti-human PD1 antibody or antigen binding fragment thereof comprising a light chain variable region comprising SEQ ID NO: 4 and a heavy chain variable region comprising SEQ ID NO: 9. In another aspect, the formulations comprise an anti-human PD1 antibody or antigen binding fragment thereof comprising a light chain comprising SEQ ID NO: 5 and a heavy chain comprising SEQ ID NO: 10. In one aspect of any of the formulations described above, the anti-human PD-1 antibody or antigen binding fragment thereof is pembrolizumab. In another aspect, the anti-human PD-1 antibody or antigen binding fragment thereof is nivolumab.

In one aspect of any of the co-formulations described above, the formulation comprises (i) an anti-CTLA4 antibody or antigen-binding fragment thereof comprising three light chain CDRs and three heavy chain CDRs, wherein the light chain CDRs comprise CDRL1 of SEQ ID NO: 38, CDRL2 of SEQ ID NO:39, CDRL3 of SEQ ID NO:40 and the heavy chain CDRs comprise CDRH1 of SEQ ID NO: 35, CDRH2 of SEQ ID NO: 36, and CDHR3 of SEQ ID NO: 37 and (ii) an anti-human PD-1 antibody or antigen binding fragment thereof comprising three light chain CDRs and three heavy chain CDRs, wherein the light chain CDRs comprise CDRL1 of SEQ ID NO: 1, CDRL2 of SEQ ID NO:2, CDRL3 of SEQ ID NO:3 and the heavy chain CDRs comprise CDRH1 of SEQ ID NO: 6, CDRH2 of SEQ ID NO: 7, and CDHR3 of SEQ ID NO: 8.

In one aspect of any of the co-formulations described above, the formulation comprises (i) an anti-CTLA4 antibody or antigen binding fragment thereof comprising a heavy chain variable region comprising SEQ ID NO: 88 and a light chain variable region comprising SEQ ID NO: 48 and (ii) an anti-human PD1 antibody or antigen binding fragment thereof comprising a light chain variable region comprising SEQ ID NO: 4 and a heavy chain variable region comprising SEQ ID NO: 9.

In another aspect of any of the co-formulations described above, the formulation comprises (i) an anti-CTLA4 antibody or antigen binding fragment thereof comprising a heavy chain comprising SEQ ID NO: 99 and a light chain comprising SEQ ID NO: 100 and (ii) an anti-human PD1 antibody or antigen binding fragment thereof comprising a light chain comprising SEQ ID NO: 5 and a heavy chain comprising SEQ ID NO: 10.

In one embodiment, the formulation is contained in a glass vial. In another embodiment, the formulation is contained in an injection device. In another embodiment, the formulation is a liquid formulation. In one aspect, the formulation is frozen to at least below-70° C. In another embodiment, the formulation is a reconstituted solution from a lyophilized formulation.

In one aspect, provided are methods of treating chronic infection or cancer in a mammalian subject (e.g., a human) in need thereof comprising: administering an effective amount of the anti-CTLA4 formulation or the co-formulation set forth herein.

### BRIEF DESCRIPTION OF THE DRAWINGS

FIG. 1A shows the UV A350 absorbance of the formulation A1 at 5°, 25°, and 40° C. over 8 weeks. FIG. 1B shows the UV A350 absorbance of the formulation A2 at 5°, 25°, and 40° C. over 8 weeks.

FIG. 2 shows the UV A350 absorbance of the formulations A1 and A2 for freeze thaw, agitation and light stress studies.

FIGS. 3A and 3B show % HMW, as determined by UP-SEC, vs. Time data at 5°, 25°, and 40° C. storage conditions for Formulations A1 and A2, respectively.

FIGS. 4A and 4B show % monomer, as determined by UP-SEC, vs. Time data at 5°, 25°, and 40° C. storage conditions for Formulations A1 and A2, respectively.

FIG. 5 shows % HMW, as determined by UP-SEC, for Formulations A1 and A2 for freeze thaw, agitation and light stress studies.

FIG. 6 shows % monomer, as determined by UP-SEC, for Formulations A1 and A2 for freeze thaw, agitation and light stress studies.

FIGS. 7A and 7B shows % Acidic, as determined by HP-IEX, vs. Time data at 5°, 25°, and 40° C. storage conditions for Formulations A1 and A2, respectively.

FIGS. 8A and 8B shows % Basic, as determined by HP-IEX, vs. Time data at 5°, 25°, and 40° C. storage conditions for Formulations A1 and A2, respectively.

FIGS. 9A and 9B shows % Main, as determined by HP-IEX, vs. Time data at 5°, 25°, and 40° C. storage conditions for Formulations A1 and A2, respectively.

FIG. 10 shows % Acidic, as determined by HP-IEX, for Formulations A1 and A2 for freeze thaw, agitation and light stress studies.

FIG. 11 shows % Basic, as determined by HP-IEX, for Formulations A1 and A2 for freeze thaw, agitation and light stress studies.

FIG. 12 shows % Main, as determined by HP-IEX, for Formulations A1 and A2 for freeze thaw, agitation and light stress studies.

FIG. 13 shows the percent oxidation of LC-M4 (methionine oxidation) as determined by peptide mapping for formulations A1 and A2.

FIG. 14 shows the percent oxidation of HC-M34 (methionine oxidation) as determined by peptide mapping for formulations A1 and A2.

FIG. 15 shows the percent oxidation of HC-M250 (methionine oxidation) as determined by peptide mapping for formulations A1 and A2.

FIG. 16 shows the percent oxidation of HC-M426 (methionine oxidation) as determined by peptide mapping for formulations A1 and A2.

FIG. 17A shows the UV A350 absorbance of the formulation B1 at 5°, 25°, and 40° C. over 8 weeks. FIG. 17B shows the UV A350 absorbance of the formulation B2 at 5°, 25°, and 40° C. over 8 weeks.

FIG. 18 shows the UV A350 absorbance of the formulations B1 and B2 for freeze thaw, agitation and light stress studies.

FIGS. 19A and 19B show % HMW, as determined by UP-SEC, vs. Time data at 5°, 25°, and 40° C. storage conditions for Formulations B1 and B2, respectively.

FIGS. 20A and 20B show % monomer, as determined by UP-SEC, vs. Time data at 5°, 25°, and 40° C. storage conditions for Formulations B1 and B2, respectively.

FIG. 21 shows % HMW, as determined by UP-SEC, for Formulations B1 and B2 for freeze thaw, agitation and light stress studies.

FIG. 22 shows % monomer, as determined by UP-SEC, for Formulations B1 and B2 for freeze thaw, agitation and light stress studies.

FIGS. 23A and 23B shows % Acidic, as determined by HP-IEX, vs. Time data at 5°, 25°, and 40° C. storage conditions for Formulations B1 and B2, respectively.

FIGS. 24A and 24B shows % Basic, as determined by HP-IEX, vs. Time data at 5°, 25°, and 40° C. storage conditions for Formulations B1 and B2, respectively.

FIGS. 25A and 25B shows % Main, as determined by HP-IEX, vs. Time data at 5°, 25°, and 40° C. storage conditions for Formulations B1 and B2, respectively.

FIG. 26 shows % Acidic, as determined by HP-IEX, for Formulations B1 and B2 for freeze thaw, agitation and light stress studies.

FIG. 27 shows % Basic, as determined by HP-IEX, for Formulations B1 and B2 for freeze thaw, agitation and light stress studies.

FIG. 28 shows % Main, as determined by HP-IEX, for Formulations B1 and B2 for freeze thaw, agitation and light stress studies.

FIG. 29 shows the percent oxidation of LC-M4 (methionine oxidation) as determined by peptide mapping for formulations B1 and B2.

FIG. 30 shows the percent oxidation of HC-M34 (methionine oxidation) as determined by peptide mapping for formulations B1 and B2.

FIG. 31 shows the percent oxidation of HC-M250 (methionine oxidation) as determined by peptide mapping for formulations B1 and B2.

FIG. 32 shows the percent oxidation of HC-M426 (methionine oxidation) as determined by peptide mapping for formulations B1 and B2.

FIG. 33 shows the KD data for the co-formulations, indicating that the formulations are stable at three different pH values (5.0, 5.5, and 6.0).

FIG. 34 shows amino acid sequences of the heavy and light chains for ipilimumab (SEQ ID NOs: 84 and 85, respectively).

### DETAILED DESCRIPTION OF THE INVENTION

In one aspect, the invention provides formulations comprising anti-CTLA4 antibodies and antigen-binding fragments thereof comprising methionine. Also provided are co-formulations of an anti-CTLA4 antibody or antigen binding fragment thereof and an anti-human PD-1 antibody or antigen binding fragment thereof comprising methionine. In each case, the formulation and co-formulation optionally comprises a chelating agent.

### I. Definitions and Abbreviations

As used throughout the specification and appended claims, the following abbreviations apply:
API active pharmaceutical ingredient
CDR complementarity determining region in the immunoglobulin variable regions, defined using the Kabat numbering system, unless otherwise indicated
CHO Chinese hamster ovary
CI confidence interval
CTLA4 cytotoxic T lymphocyte associated antigen 4
DTPA diethylenetriaminepentaacetic acid
EC50 concentration resulting in 50% efficacy or binding
ELISA enzyme-linked immunosorbant assay
FFPE formalin-fixed, paraffin-embedded
FR framework region
HRP horseradish peroxidase
HNSCC head and neck squamous cell carcinoma
IC50 concentration resulting in 50% inhibition
IgG immunoglobulin G
ICH International Conference of Harmonization
IHC immunohistochemistry or immunohistochemical mAb monoclonal antibody
MES 2-(N-morpholino) ethanesulfonic acid
NCBI National Center for Biotechnology Information
NSCLC non-small cell lung cancer
PCR polymerase chain reaction
PD-1 programmed death 1 (a.k.a. programmed cell death-1 and programmed death receptor 1)
PD-L1 programmed cell death 1 ligand 1
PD-L2 programmed cell death 1 ligand 2
PS80 polysorbate 80
TNBC triple negative breast cancer
V_{H} immunoglobulin heavy chain variable region
VK immunoglobulin kappa light chain variable region
V_{L} immunoglobulin light chain variable region
v/v volume per volume
WFI water for injection
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the invention to a subject having an immune condition or cancerous condition, or diagnosed with a cancer or pathogenic infection (e.g., viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, inhibiting the growth or survival of tumor cells, eliminating or reducing the size of one or more cancerous lesions or tumors, decreasing the level of one or more tumor markers, ameliorating, reducing the severity or duration of the cancer, prolonging the survival of a patient relative to the expected survival in a similar untreated patient.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the invention is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the present invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient.

The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic antibody.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human CTLA4 or human PD-1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

The term “pharmaceutically effective amount” or “effective amount” means an amount whereby sufficient therapeutic composition or formulation is introduced to a patient to treat a diseased or condition. One skilled in the art recognizes that this level may vary according the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of +0.1%, 0.5%, 1%, 2%, 3%, 4%, 5%, or 10%.

As used herein, “x % (w/v)” is equivalent to x g/100 ml (for example 5% w/v equals 50 mg/ml).

Formulations of the present invention include antibodies and fragments thereof that are biologically active when reconstituted or in liquid form.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Chothia” means an antibody numbering system described in A1-Lazikani et al., JMB 273:927-948 (1997).

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

A “growth inhibitory agent” when used herein refers to a compound or composition which inhibits growth of a cell, especially cancer cell over expressing any of the genes identified herein, either in vitro or in vivo. Thus, the growth inhibitory agent is one which significantly reduces the percentage of cells over expressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine) taxanes, and topo II inhibitors such as doxorubicin, epirubicin, daunorubicin, and etoposide. Those agents that arrest G1 also spill over into S-phase arrest, for example, DNA alkylating agents such as dacarbazine, mechlorethamine, and cisplatin. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995).

The terms “CTLA4 binding fragment,” “antigen binding fragment thereof,”, “binding fragment thereof” or “fragment thereof” encompass a fragment or a derivative of an antibody that still substantially retains its biological activity of binding to antigen (human CTLA4) and inhibiting its activity (e.g., blocking the binding of human CTLA4 to its native ligands). Therefore, the term “antibody fragment” or CTLA4 binding fragment refers to a portion of a full length antibody, generally the antigen binding or variable region thereof. Examples of CTLA4 antibody fragments include Fab, Fab′, F(ab′)₂, and Fv fragments. Typically, a binding fragment or derivative retains at least 10% of its CTLA4 inhibitory activity. In some embodiments, a binding fragment or derivative retains at least 25%, 50%, 60%, 70%, 80%, 90%, 95%, 99% or 100% (or more) of its CTLA4 inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 10⁹ liters/mol, as determined, e.g., by Scatchard analysis. Munsen et al. (1980) Analyt. Biochem. 107:220-239. It is also intended that a CTLA4 binding fragment can include variants having conservative amino acid substitutions that do not substantially alter its biologic activity.

The terms “PD-1 binding fragment,” “antigen binding fragment thereof,” “binding fragment thereof” or “fragment thereof” encompass a fragment or a derivative of an antibody that still substantially retains its biological activity of binding to antigen (human PD-1) and inhibiting its activity (e.g., blocking the binding of PD-1 to PDL1 and PDL2). Therefore, the term “antibody fragment” or PD-1 binding fragment refers to a portion of a full length antibody, generally the antigen binding or variable region thereof. Examples of antibody fragments include Fab, Fab′, F(ab′)₂, and Fv fragments. Typically, a binding fragment or derivative retains at least 10% of its PD-1 inhibitory activity. In some embodiments, a binding fragment or derivative retains at least 25%, 50%, 60%, 70%, 80%, 90%, 95%, 99% or 100% (or more) of its PD-1 inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 10⁹ liters/mol, as determined, e.g., by Scatchard analysis. Munsen et al. (1980) Analyt. Biochem. 107:220-239. It is also intended that a PD-1 binding fragment can include variants having conservative amino acid substitutions that do not substantially alter its biologic activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

“Fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only. A fully human antibody may be generated in a human being, in a transgenic animal having human immunoglobulin germline sequences, by phage display or other molecular biological methods.

“Hypervariable region” refers to the amino acid residues of an antibody that are responsible for antigen-binding. The hypervariable region comprises amino acid residues from a “complementarity determining region” or “CDR” (e.g., residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196:901-917). As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

“Conservatively modified variants” or “conservative substitution” refers to substitutions of amino acids are known to those of skill in this art and may be made generally without altering the biological activity of the resulting molecule, even in essential regions of the polypeptide. Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

The term “buffer” encompasses those agents which maintain the solution pH of the formulations of the invention in an acceptable range, or, for Lyophilized formulations of the invention, provide an acceptable solution pH prior to lyophilization.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g., parenteral administration), and may optionally be suitable for subcutaneous administration.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e., % high molecular weight species as determined, for example, by HP-SEC.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

### II. Formulations and Co-Formulations of the Invention.

In one aspect, the invention provides stable biological formulations comprising anti-CTLA4 antibodies or antigen binding fragments thereof which specifically bind to human CTLA4 as the active pharmaceutical ingredient. Inclusion of methionine in such formulations reduces the oxidation of methionine residues present in Fc region of the anti-CTLA4 antibody.

In one aspect, the invention also provides a co-formulation of an anti-CTLA4 antibody with an anti-PD-1 antibody. The major degradation pathways of pembrolizumab included oxidation of methionine 105 (Met105) in the heavy chain CDR3 (e.g., M105 of SEQ ID NO: 10) upon peroxide stress and oxidation of Met105 and Fc methionine residues when exposed to light. Pembrolizumab maintained its bioactivity under most stress conditions for the degradation levels tested. However, reduction in affinity to PD-1 was observed for peroxide stressed samples by Surface Plasmon Resonance (SPR). An exposed methionine residue or a methionine residue in the CDR of an antibody has the potential of impacting the biological activity of the antibody through oxidation. The addition of methionine is able to reduce oxidation of Met105 within the pembrolizumab heavy chain CDR.

### Anti-PD-1 Antibodies and Antigen-Binding Fragments Thereof

In one aspect, the invention provides stable biological formulations comprising anti-CTLA4 antibodies or antigen binding fragments thereof, co-formulated with an anti-human PD-1 antibodies or antigen binding fragments thereof which specifically bind to human PD-1 (e.g., a human or humanized anti-PD-1 antibody) as the active pharmaceutical ingredient (PD-1 API), as well as methods for using the formulations of the invention. Any anti-PD-1 antibody or antigen binding fragment thereof can be used in the co-formulations and methods of the invention. In particular embodiments, the PD-1 API is an anti-PD-1 antibody, which is selected from pembrolizumab and nivolumab. In specific embodiments, the anti-PD-1 antibody is pembrolizumab. In alternative embodiments, the anti-PD-1 antibody is nivolumab. Table 2 provides amino acid sequences for exemplary anti-human PD-1 antibodies pembrolizumab and nivolumab. Alternative PD-1 antibodies and antigen-binding fragments that are useful in the co-formulations and methods of the invention are shown in Table 3.

As used herein, “Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA for the treatment of patients with unresectable or metastatic melanoma and for the treatment of certain patients with recurrent or metastatic head and neck squamous cell cancer (HNSCC), classical Hodgkin lymphoma (cHL), urothelial carcinoma, gastric cancer, microsatellite instability-high (MSI-H) cancer and non-small cell lung cancer, as described in the Prescribing Information for KEYTRUDA™ (Merck & Co., Inc., Whitehouse Station, NJ USA; initial U.S. approval 2014, updated September 2017).

In some embodiments, an anti-human PD-1 antibody or antigen binding fragment thereof for use in the co-formulations of the invention comprises three light chain CDRs of CDRL1, CDRL2 and CDRL3 and/or three heavy chain CDRs of CDRH1, CDRH2 and CDRH3.

In one embodiment of the invention, CDRL1 is SEQ ID NO: 1 or a variant of SEQ ID NO: 1, CDRL2 is SEQ ID NO:2 or a variant of SEQ ID NO:2, and CDRL3 is SEQ ID NO: 3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO:8.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO:13.

In one embodiment, CDRH1 is SEQ ID NO:16 or a variant of SEQ ID NO:16, CDRH2 is SEQ ID NO:17 or a variant of SEQ ID NO:17, and CDRH3 is SEQ ID NO:18 or a variant of SEQ ID NO:18.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment, the three light chain CDRs are SEQ ID NO:11, SEQ ID NO: 12, and SEQ ID NO: 13 and the three heavy chain CDRs are SEQ ID NO:16, SEQ ID NO: 17 and SEQ ID NO:18.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO: 24, CDRH2 is SEQ ID NO: 25 or a variant of SEQ ID NO:25, and CDRH3 is SEQ ID NO: 26 or a variant of SEQ ID NO:26.

In another embodiment, the three light chain CDRs are SEQ ID NO:21, SEQ ID NO: 22, and SEQ ID NO:23 and the three heavy chain CDRs are SEQ ID NO:24, SEQ ID NO: 25 and SEQ ID NO:26.

Some anti-human PD-1 antibody and antigen binding fragments of the invention comprise a light chain variable region and a heavy chain variable region. In some embodiments, the light chain variable region comprises SEQ ID NO:4 or a variant of SEQ ID NO: 4, and the heavy chain variable region comprises SEQ ID NO:9 or a variant of SEQ ID NO: 9. In further embodiments, the light chain variable region comprises SEQ ID NO:14 or a variant of SEQ ID NO:14, and the heavy chain variable region comprises SEQ ID NO:19 or a variant of SEQ ID NO:19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO: 29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the co-formulations of the invention, the anti-human PD-1 antibody or antigen binding fragment comprises a light chain variable region comprising or consisting of SEQ ID NO:4 and a heavy chain variable region comprising or consisting SEQ ID NO: 9. In a further embodiment, the anti-human PD-1 antibody or antigen binding fragment comprises a light chain variable region comprising or consisting of SEQ ID NO:14 and a heavy chain variable region comprising or consisting of SEQ ID NO:19. In one embodiment of the formulations of the invention, the anti-human PD-1 antibody or antigen binding fragment comprises a light chain variable region comprising or consisting of SEQ ID NO: 28 and a heavy chain variable region comprising or consisting SEQ ID NO:27. In a further embodiment, the anti-human PD-1 antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO: 27.

In another embodiment, the co-formulations of the invention comprise an anti-human PD-1 antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment, the anti-human PD-1 antibody or antigen binding protein of the co-formulations of the invention comprises V_{L} and V_{H} domains having up to 1, 2, 3, 4, or 5 or more amino acid substitutions, and exhibits specific binding to PD-1.

In any of the embodiments above, the PD-1 API may be a full-length anti-PD-1 antibody or an antigen binding fragment thereof that specifically binds human PD-1. In certain embodiments, the PD-1 API is a full-length anti-PD-1 antibody selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the PD-1 API is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the PD-1 API is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 API is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 API is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 API is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some co-formulations of the invention, the PD-1 API is pembrolizumab or a pembrolizumab biosimilar. In some co-formulations of the invention, the PD-1 API is nivolumab or a nivolumab biosimilar.

Ordinarily, amino acid sequence variants of the anti-PD-1 antibodies and antigen binding fragments of the invention or the anti-CTLA4 antibodies and antigen binding fragments of the invention will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 2
Exemplary PD-1 Antibody Sequences

| | | |
|---|---|---|
| | | SEQ |
| Antibody | | ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYL | 4 |
| Region | HWYQQKPGQAPRLLIYLASYLESGVPARFSGSGSGTD | |
| | FTLTISSLEPEDFAVYYCQHSRDLPLTFGGGTKVEIK | |
| Light | EIVLTQSPATLSLSPGERATLSCRASKGVSTSGYSYL | 5 |
| Chain | HWYQQKPGQAPRLLIYLASYLESGVPARFSGSGSGTD | |
| | FTLTISSLEPEDFAVYYCQHSRDLPLTFGGGTKVEIK | |
| | RTVAAPSVFIFPPSDEQLKSGTASVVCLLNNFYPREA | |
| | KVQWKVDNALQSGNSQESVTEQDSKDSTYSLSSTLTL | |
| | SKADYEKHKVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDS | |
| | STTTAYMELKSLQFDDTAVYYCARRDYRFDMGFDYWG | |
| | QGTTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDS | |
| | STTTAYMELKSLQFDDTAVYYCARRDYRFDMGFDYWG | |
| | QGTTVTVSSASTKGPSVFPLAPCSRSTSESTAALGCL | |
| | VKDYFPEPVTVSWNSGALTSGVHTFPAVLQSSGLYSL | |
| | SSVVTVPSSSLGTKTYTCNVDHKPSNTKVDKRVESKY | |
| | GPPCPPCPAPEFLGGPSVFLFPPKPKDTLMISRTPEV | |
| | TCVVVDVSQEDPEVQFNWYVDGVEVHNAKTKPREEQF | |
| | NSTYRVVSVLTVLHQDWLNGKEYKCKVSNKGLPSSIE | |
| | KTISKAKGQPREPQVYTLPPSQEEMTKNQVSLTCLVK | |
| | GFYPSDIAVEWESNGQPENNYKTTPPVLDSDGSFFLY | |
| | SRLTVDKSRWQEGNVFSCSVMHEALHNHYTQKSLSLS | |
| | LGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWYQ | 14 |
| Region | QKPGQAPRLLIYDASNRATGIPARFSGSGSGTDFTLT | |
| | ISSLEPEDFAVYYCQQSSNWPRTFGQGTKVEIK | |
| Light | EIVLTQSPATLSLSPGERATLSCRASQSVSSYLAWYQ | 15 |
| Chain | QKPGQAPRLLIYDASNRATGIPARFSGSGSGTDFTLT | |
| | ISSLEPEDFAVYYCQQSSNWPRTFGQGTKVEIKRTVA | |
| | APSVFIFPPSDEQLKSGTASVVCLLNNFYPREAKVQW | |
| | KVDNALQSGNSQESVTEQDSKDSTYSLSSTLTLSKAD | |
| | YEKHKVYACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHWV | 19 |
| Region | RQAPGKGLEWVAVIWYDGSKRYYADSVKGRFTISRDN | |
| | SKNTLFLQMNSLRAEDTAVYYCATNDDYWGQGTLVTV | |
| | SS | |
| Heavy | QVQLVESGGGVVQPGRSLRLDCKASGITFSNSGMHWV | 20 |
| Chain | RQAPGKGLEWVAVIWYDGSKRYYADSVKGRFTISRDN | |
| | SKNTLFLQMNSLRAEDTAVYYCATNDDYWGQGTLVTV | |
| | SSASTKGPSVFPLAPCSRSTSESTAALGCLVKDYFPE | |
| | PVTVSWNSGALTSGVHTFPAVLQSSGLYSLSSVVTVP | |
| | SSSLGTKTYTCNVDHKPSNTKVDKRVESKYGPPCPPC | |
| | PAPEFLGGPSVFLFPPKPKDTLMISRTPEVTCVVVDV | |
| | SQEDPEVQFNWYVDGVEVHNAKTKPREEQFNSTYRVV | |
| | SVLTVLHQDWLNGKEYKCKVSNKGLPSSIEKTISKAK | |
| | GQPREPQVYTLPPSQEEMTKNQVSLTCLVKGFYPSDI | |
| | AVEWESNGQPENNYKTTPPVLDSDGSFFLYSRLTVDK | |
| | SRWQEGNVFSCSVMHEALHNHYTQKSLSLSLGK | |

TABLE 3
Additional PD-1 Antibodies and Antigen Binding
Fragments Useful in the Co-Formulations, Methods
and Uses of the Invention.
A. Antibodies and antigen binding fragments
comprising light and
heavy chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

C. Antibodies and antigen binding fragments
comprising the mature h109A heavy chain variable
region and one of the mature K09A
light chain variable regions in WO 2008/156712

| | |
|---|---|
| Heavy chain | SEQ ID NO: 27 |
| VR | |
| Light chain | SEQ ID NO: 28 or SEQ ID NO: 29 |
| VR | or SEQ ID NO: 30 |

D. Antibodies and antigen binding fragments
comprising the mature 409 heavy chain and one of
the mature K09A light chains in WO 2008/156712

| | |
|---|---|
| Heavy chain | SEQ ID NO: 31 |
| Light chain | SEQ ID NO: 32 or SEQ ID NO: 33 |
| | or SEQ ID NO: 34 |

In some embodiments of the co-formulation of the invention, the PD-1 API (i.e., the anti-PD-1 antibody or antigen binding fragment thereof) is present in a concentration of from about 25 mg/mL to about 100 mg/mL. In alternative embodiments, the API is present in a concentration of about 10 mg/mL, about 25 mg/mL, about 50 mg/mL, about 75 mg/mL, or about 100 mg/mL.

### Anti-CTLA4 Antibodies and Antigen-Binding Fragment Thereof

The invention provides stable biological formulations comprising anti-CTLA4 antibodies or antigen binding fragments thereof which specifically bind to human CTLA4 (e.g., a human or humanized anti-CTLA4 antibody) as the active pharmaceutical ingredient (CTLA4 API), as well as methods for using the formulations of the invention.

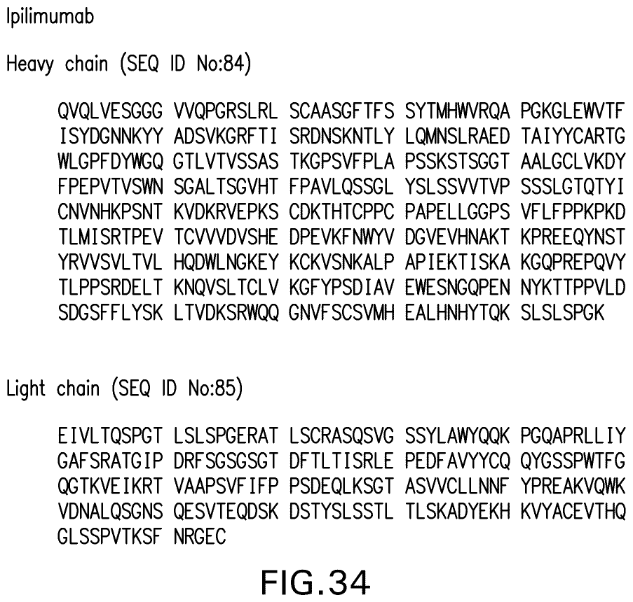

The invention also provides stable biological co-formulations comprising (i) anti-CTLA4 antibody or antigen binding fragment thereof which specifically bind to human CTLA4 (e.g., a human or humanized anti-CTLA4 antibody) and (ii) an anti-human PD-1 antibody or antigen binding fragment thereof which specifically binds to human PD-1. Any anti-CTLA4 antibody or antigen binding fragment thereof can be used in the formulation, including the co-formulation, and methods of the invention. Tables 4-8 and FIG. 34 provide amino acid sequences for exemplary anti-CTLA4 antibodies and antigen-binding fragments that are useful in the formulations, including co-formulations and methods of the invention.

In one embodiment of the formulations, including the co-formulation, the anti-CTLA-4 antibody is the human monoclonal antibody 10D1, now known as ipilimumab, and marketed as Yervoy™, which is disclosed in U.S. Pat. No. 6,984,720 and WHO Drug Information 19 (4): 61 (2005). In another embodiment, the anti-CTLA-4 antibody is tremelimumab, also known as CP-675,206) which is an IgG2 monoclonal antibody which is described in U.S. Patent Application Publication No. 2012/263677, or PCT International Application Publication Nos. WO 2012/122444 or 2007/113648 A2.

In one of the formulation, including the co-formulation, anti-CTLA-4 antibody is a monoclonal antibody that comprises a heavy chain having the amino acid sequence set forth in SEQ ID NO:84 and a light chain comprising the amino acid sequence set forth in SEQ ID NO: 85. In some embodiments, the CTLA4 antibody is an antigen binding fragment of SEQ ID NO: 84 and/or SEQ ID NO:85, wherein the antigen binding fragment specifically binds to CTLA4.

In one embodiment of the formulations, including the co-formulation, of the invention the anti-CTLA-4 antibody is any of the anti-CTLA-4 antibodies, or antigen binding fragments thereof, disclosed in International Application Publication No. WO 2016/015675 A1. In one embodiment, the anti-CTLA4 antibody is a monoclonal antibody which comprises the following CDR's:

HCDR1 comprising the amino acid sequence
(SEQ ID NO: 35)
GFTFSDNW
HCDR2 comprising the amino acid sequence
(SEQ ID NO: 36)
IRNKPYNYET
HCDR3 comprising the amino acid sequence
(SEQ ID NO: 37)
TAQFAY
and/or
LCDR1 comprising the amino acid sequence
(SEQ ID NO: 38)
ENIYGG
LCDR2 comprising the amino acid sequence
(SEQ ID NO: 39)
GAT
LCDR3 comprising an amino acid sequence
selected from:
(SEQ ID NO: 40)
QNVLRSPFT;
(SEQ ID NO: 41)
QNVLSRHPG;
OR
(SEQ ID NO: 42)
QNVLSSRPG

In one embodiment of the formulations, including the co-formulation, of the invention, the anti-CTLA4 antibody or antigen binding fragment thereof comprises a variable heavy chain and a variable light chain. In one embodiment, the variable heavy and variable light chain comprises the VH and VL sequences of 8D2/8D2 (RE) or a variant thereof. In another embodiment, the variable heavy and variable light chain comprises the VH and VL sequences of 8D2H1L1 or a variant thereof. In a further embodiment, the variable heavy chain and the variable light chain comprise the VH and VL sequences of 8D2H2L2 or a variant thereof. In another embodiment, the variable heavy chain and the variable light chain comprise the VH and the VL sequences of 8D3H3L3 or a variant thereof. In a further embodiment, the variable heavy chain and the variable light chain comprises the VH and VL sequences of 8D2H2117 or a variant thereof. In one embodiment, the methionine at position 18 of the variant of any of 8D2/8D2 (RE), 8D2H1L1, 8D2H2L2, 8D2H2L15, or 8D2H2L17 is independently substituted with an amino acid selected from: leucine, valine, isoleucine, and alanine. In another embodiment of the variant, the methionine at position 18 of the variant of any of 8D2/8D2 (RE), 8D2H1L1, 8D2H2L2, 8D2H2L15, or 8D2H2L17 is substituted with leucine.

In one embodiment of the formulation, including the co-formulation, the anti-CTLA4 antibody or antigen binding fragment thereof is 8D2H2L2 or a variant thereof, wherein the methionine at position 18 in the variable heavy (VH) chain amino acid sequence of the 8D2H2L2 variant is independently substituted with an amino acid selected from: leucine, valine, isoleucine, and alanine.

TABLE 4
Exemplary sequences of anti-CTLA4 antibodies

| Antibody | V_{H} | V_{L} |
|---|---|---|
| 8D2/8D2 | EVKLDETGGGLVQPGRPMKLSCVASGF | DIQMTQSPASLSASVGETVTITCGTSE |
| (RE) | TFSDNWMNWVRQSPEKGLEWLAQIRNK | NIYGGLNWYQRKQGKSPQLLIFGATN |
| | PYNYETYYSDSVKGRFTISRDDSKSSVY | LADGMSSRFSGSGSGRQYSLKISSLHP |
| | LQMNNLRGEDMGIYYCTAQFAYWGQG | DDVATYYCQNVLRSPFTFGSGTKLEI |
| | TLVTVSA | (SEQ ID NO: 44) |
| | (SEQ ID NO: 43) | |
| 8D2/8D2 | EVKLDETGGGLVQPGRPIKLSCVASGFT | DIQMTQSPASLSASVGETVTITCGTSE |
| RE | FSDNWMNWVRQSPEKGLEWLAQIRNKP | NIYGGLNWYQRKQGKSPQLLIFGATN |
| VARIANT | YNYETYYSDSVKGRFTISRDDSKSSVYL | LADGMSSRFSGSGSGRQYSLKISSLHP |
| 1 | QMNNLRGEDMGIYYCTAQFAYWGQGT | DDVATYYCQNVLRSPFTFGSGTKLEI |
| | LVTVSA | (SEQ ID NO: 44) |
| | (SEQ ID NO: 86) | |
| 8D2H1L1 | EVQLVESGGGLVQPGGSMRLSCAASGFT | DIQMTQSPSSLSASVGDRVTITCRT |
| | FSDNWMNWVRQAPGKGLEWLAQIRNK | SENIYGGLNWYQRKQGKSPKLLIY |
| | PYNYETYYSDSVKGRFTISRDDSKNSVY | GATNLASGMSSRFSGSGSGTDYTL |
| | LQMNSLKTEDTGVYYCTAQFAYWGQG | KISSLHPDDVATYYCQNVLRSPFTF |
| | TLVTVSS | GSGTKLEIK |
| | (SEQ ID NO: 45) | (SEQ ID NO: 46) |
| 8D2H1L1 | EVQLVESGGGLVQPGGSIRLSCAASG | DIQMTQSPSSLSASVGDRVTITCRT |
| VARIANT | FTFSDNWMNWVRQAPGKGLEWLAQ | SENIYGGLNWYQRKQGKSPKLLIY |
| 1 | IRNKPYNYETYYSDSVKGRFTISRDD | GATNLASGMSSRFSGSGSGTDYTL |
| | SKNSVYLQMNSLKTEDTGVYYCTAQ | KISSLHPDDVATYYCQNVLRSPFTF |
| | FAYWGQGTLVTVSS | GSGTKLEIK |
| | (SEQ ID NO: 87) | (SEQ ID NO: 46) |
| 8D2H2L2 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLRSPFTF |
| | QFAYWGQGTLVTVSS | GSGTKLEIK |
| | (SEQ ID NO: 47) | (SEQ ID NO: 48) |
| 8D2H2L2 | EVQLVESGGGLVQPGGSLRLSCAASG | DIQMTQSPSSLSASVGDRVTITCRT |
| VARIANT | FTFSDNWMNWVRQAPGKGLEWLAQ | SENIYGGLNWYQRKPGKSPKLLIY |
| 1 | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLRSPFTF |
| | FAYWGQGTLVTVSS | GSGTKLEIK |
| | (SEQ ID NO: 88) | (SEQ ID NO: 48) |
| 8D3H3L3 | EVQLVESGGGLVQPGGSLRLSCAASG | DIQMTQSPSSLSASVGDRVTITCRA |
| | FTFSDNWMNWVRQAPGKGLEWVAQ | SENIYGGLNWYQQKPGKAPKLLIY |
| | IRNKPYNYETEYAASVKGRFTISRDD | GATSLASGVPSRFSGSGSGTDYTLT |
| | SKNSAYLQMNSLKTEDTAVYYCTAQ | ISSLQPEDFATYYCQNVLRSPFTFG |
| | FAYWGQGTLVTVSS | SGTKLEIK |
| | (SEQ ID NO: 49) | (SEQ ID NO: 50) |
| 8D2H2L15 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSRHPGF |
| | QFAYWGQGTLVTVSS | GSGTKLEIK |
| | (SEQ ID NO: 51) | (SEQ ID NO: 52) |
| 8D2H2L15 | EVQLVESGGGLVQPGGSIRLSCAASG | DIQMTQSPSSLSASVGDRVTITCRT |
| VARIANT | FTFSDNWMNWVRQAPGKGLEWLAQ | SENIYGGLNWYQRKPGKSPKLLIY |
| 1 | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLSRHPGF |
| | FAYWGQGTLVTVSS | GSGTKLEIK |
| | (SEQ ID NO: 89) | (SEQ ID NO: 52) |
| 8D2H2L17 | EVQLVESGGGLVQPGGSMRLSCAAS | DIQMTQSPSSLSASVGDRVTITCRT |
| | GFTFSDNWMNWVRQAPGKGLEWLA | SENIYGGLNYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSSRPGF |
| | QFAYWGQGTLVTVSS | GSGTKLEIK |
| | (SEQ ID NO: 53) | (SEQ ID NO: 54) |
| QD2H2L17 | EVQLVESGGGLVQPGGSIRLSCAASG | DIQMTQSPSSLSASVGDRVTITCRT |
| VARIANT | FTFSDNWMNWVRQAPGKGLEWLAQ | SENIYGGLNWYQRKPGKSPKLLIY |
| 1 | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLSSRPGF |
| | FAYWGQGTLVTVSS | GSGTKLEIK |
| | (SEQ ID NO: 90) | (SEQ ID NO: 54) |
| Antibody | Full Heavy Chain | Full Light Chain |
| 8D2H2L2 | EVQLVESGGGLVQPGGSLRLSCAASG | DIQMTQSPSSLSASVGDRVTITCRT |
| VARIANT | FTFSDNWMNWVRQAPGKGLEWLAQ | SENIYGGLNWYQRKPGKSPKLLIY |
| 1 | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLRSPFTF |
| | FAYWGQGTLVTVSSASTKGPSVFPLA | GSGTKLEIKRTVAAPSVFIFPPSDEQ |
| | PSSKTSTGGTAALGCLVKDYFPEPVT | LKSGTASVVCLLNNFYPREAKVQ |
| | VSWNSGALTSGVHTFPAVLQSSGLYS | WKVDNALQSGNSQESVTEQDSKD |
| | LSSVVTVPSSSLGTQTYICNVNHKPS | STYSLSSTLTLSKADYEKHKVYAC |
| | NTKVDKKVEPKSCDKTHTCPPCPAPE | EVTHQGLSSPVTKSFNRGEC |
| | LLGGPSVFLFPPKPKDTLMISRTPEVT | (SEQ ID NO: 100) |
| | CVVVDVSHEDPEVKFNWYVDGVEV | |
| | HNAKTKPREEQYNSTYRVVSVLTVL | |
| | HQDWLNGKEYKCKVSNKALPAPIEK | |
| | TISKAKGQPREPQVYTLPPSRDELTK | |
| | NQVSLTCLVKGFYPSDIAVEWESNGQ | |
| | PENNYKTTPPVLDSDGSFFLYSKLTV | |
| | DKSRWQQGNVFSCSVMHEALHNHY | |
| | TQKSLSLSPGK | |
| | (SEQ ID NO: 99) | |

In another embodiment of the formulations, including the co-formulation, of the invention, the a anti-CTLA4 antibody or antigen binding fragment thereof comprises the VH and VL chain sequences of 8D2/8D2 (RE) variant 1. In a further embodiment, the anti-CTLA4 antibody or antigen binding fragment thereof comprises the VH and VL chain sequences of 8D2H1L1 variant 1. In another embodiment, the anti-CTLA4 antibody or antigen binding fragment thereof comprises the VH and VL chain sequences of 8D2H2L2 variant 1. In a further embodiment, the anti-CTLA4 antibody or antigen binding fragment thereof comprises the VH and VL chain sequences of variant of 8D2H2L15. In a another embodiment, the anti-CTLA4 antibody or antigen binding fragment thereof comprises the VH and VL chain sequences or a variant of 8D2H2117.

In one embodiment of the formulations, including the co-formulation, of the invention the anti-CTLA4 antibody is any of the anti-CTLA4 antibodies, or antigen binding fragments thereof, described in PCT International Application No. PCT/CN2016/096357, filed Aug. 23, 2016. In one embodiment, the anti-CTLA4 antibody is mouse antibody 4G10, comprising the following VH chain and VL chain amino sequences, and humanized versions of this antibody.

TABLE 5
murine anti-CTLA4 antibody

| Antibody | V_{H} | V_{L} |
|---|---|---|
| 4G10 | QVKLQESGPELVKPGASM | QAVVTQESALTTSPGETVT |
| murine | KISCKASGYSFTGYTMNW | LTCRSSTGAVTTSNFANWV |
| | VKQSHGKNLEWIGLINPY | QEKPDHLFTSLIGGTNNRA |
| | NNITNYNQKFMGKATFTV | PGVPARFSGSLIGDKAALT |
| | DKSSSTAYMELLRLTSED | ITGAQTEDEAIYFCALWYS |
| | SGVYFCARLDYRSYWGQG | NHWVFGGGTKLTVLGQPKS |
| | TLVTVSA | SPSVTLFQGQFC |
| | (SEQ ID NO: 55) | (SEQ ID NO: 56) |

In one embodiment of the formulations, including the co-formulation, of the invention, the anti-CTLA4 antibody is a monoclonal antibody which comprises the following CDR's:
HCDR1 comprising the amino acid sequence selected from GYSFTGYT (SEQ ID NO: 57) or GYTX₁N (SEQ ID NO:58), wherein X₁ is M, V, L, I, G, A, S, T.
HCDR2 comprising the amino acid sequence selected from INPYNX₁IX₂, (SEQ ID NO: 59) wherein X₁ is N, D or E, and X₂ is T, D, E, G or A; or LINPYNX₁IX₂NYX₃QKFX₄G (SEQ ID NO:60), wherein X₁ is N, D; X₂ is T, D, E, G, or A; X₃ is A or N; and X₄ is Q or M.
HCDR3 comprising the amino acid sequence selected from LDYRSY (SEQ ID NO: 61) or ARLDYRSY (SEQ ID NO:62) and/or
LCDR1 comprising the amino acid sequence selected from TGAVTTSNF (SEQ ID NO: 63), or GSSTGAVTTSNFXIN (SEQ ID NO:64), wherein X₁ is P or A;
LCDR2 comprising the amino acid sequence selected from GTN, or GTNNX₁AX₂ (SEQ ID NO:65), wherein X₁ is K, R or any amino acid except M or C; and X₂ is S or P;
LCDR3 comprising an amino acid sequence selected from ALX₁YSNHX₂ (SEQ ID NO: 66), wherein X₁ is W or any amino acid except M or C and X₂ is W or any amino acid except M or C; or ALX₁YSNHX₂V (SEQ ID NO:67) wherein X₁ is W or any amino acid except M or C and X₂ is W or any amino acid except M or C.

In another embodiment, the humanized VH sequences of the 4G10 antibody comprises any of the following VH sequences:

TABLE 6
Exemplary anti-CTLA4 antibody sequences

| Antibody | V_{H} |
|---|---|
| 4G10H1 | QVQLVESGAELVKPGASMKISCKASGYSFTGYTMNW |
| humanized | VKQAPGQGLEWIGLINPYNNITNYNQKFMGKATFTV |
| | DKSISTAYMELSRLTSDDSGVYFCARLDYRSYWGQG |
| | TLVTVSA |
| | (SEQ ID NO: 68) |
| 4G10H3 | QVQLVESGAEVKKPGASVKVSCKASGYSFTGYTMNW |
| humanized | VRQAPGQGLEWIGLINPYNNITNYAQKFQGRVTFTV |
| | DTSISTAYMELSRLRSDDTGVYFCARLDYRSYWGQG |
| | TLVTVSA |
| | (SEQ ID NO: 69) |
| 4G10H4 | QVQLVESGAEVKKPGASVKVSCKASGYSFTGYTMNW |
| humanized | VRQAPGQGLEWIGLINPYNDITNYAQKFQGRVTFTV |
| | DTSISTAYMELSRLRSDDTGVYFCARLDYRSYWGQG |
| | TLVTVSA |
| | (SEQ ID NO: 70) |
| 4G10H5 | QVQLVESGAEVKKPGASVKVSCKASGYSFTGYTMNW |
| humanized | VRQAPGQGLEWIGLINPYNNIDNYAQKFQGRVTFTV |
| | DTSISTAYMELSRLRSDDTGVYFCARLDYRSYWGQG |
| | TLVTVSA |
| | (SEQ ID NO: 71) |
| 4G10H | QVQLVESGAEX₁KKPGASX₂KX₃SCKASGYSFTGYT |
| consensus | X₄NWVX₅QAPGQGLEWIGLINPYNX₆IX₇NYX₈QKF |
| humanized | X₉GX₁₀X₁₁TFTVDX₁₂SISTAYMELSRLX₁₃SDDX₁₄ |
| | GVYFCARLDYRSYWGQGTLVTVSA |
| | (SEQ ID NO: 72) |
| | X₁ = V or L |
| | X₂ = V or M |
| | X₃ = V or I |
| | X₄ = M, V, L, I, G, A, S, T |
| | X₅ = R or K |
| | X₆ = N or D or E |
| | X₇ = T or D or E or G or A |
| | X₈ = A or N |
| | X₉ = Q or M |
| | X₁₀ = R or K |
| | X₁₁ = V or A |
| | X₁₂ = T or K |
| | X₁₃ = R or T |
| | X₁₄ = T or S |

In other embodiments of the formulations, including the co-formulation, of the invention, the humanized VL sequences of the 4G10 antibody comprises any of the following VL sequences:

TABLE 7
Exemplary anti-CTLA4 antibody sequences

| Antibody | V_{L} |
|---|---|
| 4G10L1 | QAVVTQEPSLTVSPGGTVTLTCGSSTGAVTT |
| humanized | SNFANWVQEKPGQAFRSLIGGTNNRASWVPA |
| | RFSGSLLGGKAALTISGAQPEDEAEYFCALW |
| | YSNHWVFGGGTKLTVL |
| | (SEQ ID NO: 73) |
| 4G10L3 | QAVVTQEPSLTVSPGGTVTLTCGSSTGAVTT |
| humanized | SNFPNWVQQKPGQAPRSLIGGTNNKASWTPA |
| | RFSGSLLGGKAALTISGAQPEDEAEYYCALW |
| | YSNHWVFGGGTKLTVL |
| | (SEQ ID NO: 74) |
| 4G10Lconsensus | QAVVTQEPSLTVSPGGTVTLTCGSSTGAVTT |
| humanized | SNFX₁NWVQX₂KPGQAX₃RSLIGGTNNX₄AX₅W |
| | X₆PARFSGSLLGGKAALTISGAQPEDEAEYX₇ |
| | CALX₈YSNHX₉VFGGGTKLTVL |
| | (SEQ ID NO: 75) |
| | X₁ = P or A |
| | X₂ = Q or E |
| | X₃ = P or F |
| | X₄ = K or R or any other amino |
| | acid except for M or C |
| | X₅ = S or P |
| | X₆ = T or V |
| | X₇ = Y or F |
| | X₈ = W or any amino acid except |
| | M or C |
| | X₉ = W or any amino acid except |
| | M or C |

In some embodiments, the anti-CTLA4 antibody comprises a variable heavy chain and a variable light chain sequence corresponding to the VH and VL sequence of 4G10H1L1. In another embodiment, the anti-CTLA4 antibody comprises a variable heavy chain and a variable light chain sequence corresponding to the VH and VL sequence of 4G10H3L3. In one embodiment, the anti-CTLA4 antibody comprises a variable heavy chain and a variable light chain sequence corresponding to the VH and VL sequence of 4G10H3L3. In another embodiment, the anti-CTLA4 antibody comprises a variable heavy chain and a variable light chain sequence corresponding to the VH and VL sequence of 4G10H5L3.

TABLE 8
Exemplary anti-CTLA4 antibody sequences

| Antibody | V_{H} | V_{L} |
|---|---|---|
| 4G10H1L1 | QVQLVESGAELVKPGASM | QAVVTQEPSLTVSPGGT |
| | KISCKASGYSFTGYTMNW | VTLTCGSSTGAVTTSNF |
| | VKQAPGQGLEWIGLINPY | ANWVQEKPGQAFRSLIG |
| | NNITNYNQKFMGKATFTV | GTNNRASWVPARFSGSL |
| | DKSISTAYMELSRLTSDD | LGGKAALTISGAQPEDE |
| | SGVYFCARLDYRSYWGQG | AEYFCALWYSNHWVFGG |
| | TLVTVSA | GTKLTVL |
| | (SEQ ID NO: 76) | (SEQ ID NO: 77) |
| 4G10H3L3 | QVQLVESGAEVKKPGASV | QAVVTQEPSLTVSPGGT |
| | KVSCKASGYSFTGYTMNW | VTLTCGSSTGAVTTSNF |
| | VRQAPGQGLEWIGLINPY | PNWVQQKPGQAPRSLIG |
| | NNITNYAQKFQGRVTFTV | GTNNKASWTPARFSGSL |
| | DTSISTAYMELSRLRSDD | LGGKAALTISGAQPEDE |
| | TGVYFCARLDYRSYWGQG | AEYYCALWYSNHWVFGG |
| | TLVTVSA | GTKLTVL |
| | (SEQ ID NO: 78) | (SEQ ID NO: 79) |
| 4G10H4L3 | QVQLVESGAEVKKPGASV | QAVVTQEPSLTVSPGGT |
| | KVSCKASGYSFTGYTMNW | VTLTCGSSTGAVTTSNF |
| | VRQAPGQGLEWIGLINPY | PNWVQQKPGQAPRSLIG |
| | NDITNYAQKFQGRVTFTV | GTNNKASWTPARFSGSL |
| | DTSISTAYMELSRLRSDD | LGGKAALTISGAQPEDE |
| | TGVYFCARLDYRSYWGQG | AEYYCALWYSNHWVFGG |
| | TLVTVSA | GTKLTVL |
| | (SEQ ID NO: 80) | (SEQ ID NO: 81) |
| 4G10H5L3 | QVQLVESGAEVKKPGASV | QAVVTQEPSLTVSPGGT |
| | KVSCKASGYSFTGYTMNW | VTLTCGSSTGAVTTSNF |
| | VRQAPGQGLEWIGLINPY | PNWVQQKPGQAPRSLIG |
| | NNIDNYAQKFQGRVTFTV | GTNNKASWTPARFSGSL |
| | DTSISTAYMELSRLRSDD | LGGKAALTISGAQPEDE |
| | TGVYFCARLDYRSYWGQG | AEYYCALWYSNHWVFGG |
| | TLVTVSA | GTKLTVL |
| | (SEQ ID NO: 82) | (SEQ ID NO: 83) |

TABLE 9
Additional anti-human CTLA4 antibodies

| A. Comprises light and heavy chain | |
|---|---|
| CDRs of Ipilimumab | |
| CDRL1 | RASQSVGSSYLA (SEQ ID NO: 91) |
| CDRL2 | GAFSRAT (SEQ ID NO: 92) |
| CDRL3 | QQYGSSPWT (SEQ ID NO: 93) |
| CDRH1 | SYTMH (SEQ ID NO: 94) |
| CDRH2 | FISYDGNNKYYADSVKG (SEQ ID NO: 95) |
| CDRH3 | TGWLGPFDY (SEQ ID NO: 96) |
| C. Comprises the mature heavy chain variable | |
| region and the mature light chain variable | |
| region of Ipilimumab | |
| Heavy | QVQLVESGGGVVQPGRSLRLSCAASGFTFSSYTMHWVRQA |
| chain | PGKGLEWVTFISYDGNNKYYADSVKGRFTISRDNSKNTLY |
| VR | LQMNSLRAEDTAIYYCARTGWLGPFDYWGQGTLVTVSS |
| | (SEQ ID NO: 97) |
| Light | EIVLTQSPGT LSLSPGERATLSCRASQSVGSSYLAWYQQK |
| chain | PGQAPRLLIYGAFSRATGIPDRFSGSGSGTDFTLTISRLE |
| VR | PEDFAVYYCQQYGSSPWTFGQGTKVEIK |
| | (SEQ ID NO: 98) |
| D. Comprises the mature heavy chain and the | |
| mature light chain of Ipilimumab | |
| Heavy | SEQ ID NO: 84 |
| chain | |
| Light | SEQ ID NO: 85 |
| chain | |

In another embodiment of the formulations, including the co-formulation, of the invention, the anti-CTLA-4 antibody is an antibody, or antigen binding fragment thereof, which cross-competes for binding to human CTLA-4 with, or binds to the same epitope region of human CTLA-4 as does ipilimumab, tremelimumab, or any of the above described antibodies, including 8D2/8D2 (RE) or 8D2/8D2 (RE) variant 1, 8D2H1L1 or 8D2H1L1 variant 1, 8D2H2L2 or 8D2H2L2 variant 1, 8D3H3L3, 8D2H2L15 or 8D2H2L15 variant 1 thereof, 8D2H2L17 or 8D2H2L17 variant 1, 4G10H1L1 or variant thereof, 4G10H3L3 or variant thereof, 4G10H3L3 or variant thereof, and 4G10H5L3 or variant thereof.

### Formulations

In some aspects of the invention, the formulations described herein minimize the formation of antibody aggregates (high molecular weight species) and particulates, high and low molecular weight species, minimize oxidation of methionine residues, and insure that the antibody retains biological activity over time.

In one aspect, the invention includes various formulations of an anti-CTLA4 antibody, or antigen binding fragment thereof. For example, the present invention includes formulations comprising (i) an anti-CTLA4 antibody or antigen binding fragment thereof, (ii) a buffer (e.g., L-histidine or acetate), (iii) a non-reducing sugar (e.g., sucrose); (iv) a non-ionic surfactant (e.g., polysorbate 80); and (v) an antioxidant (e.g., L-methionine). In one aspect, the formulation further comprises an anti-PD-1 antibody. In one aspect, the formulation may further comprise a chelator. In one embodiment, the chelator is diethylenetriaminepentaacetic acid (DTPA).

In one aspect, the invention also includes various co-formulations of an anti-CTLA4 antibody, or antigen binding fragment thereof and an anti-human PD-1 antibody, or antigen binding fragment thereof. In one embodiment, the present invention includes formulations comprising (i) an anti-CTLA4 antibody, or antigen binding fragment thereof, (ii) an anti-human PD-1 antibody or antigen binding fragment thereof, (iii) a buffer (e.g., L-histidine or acetate), (iv) a non-reducing sugar (e.g., sucrose), (v) a non-ionic surfactant (e.g., polysorbate 80), and (vi) an antioxidant (e.g., L-methionine). In one embodiment, the formulation may further comprise a chelator (e.g., DTPA).

Pharmaceutical formulations described herein may include buffers. The term “buffer” encompasses those agents which maintain the solution pH of the liquid formulations described herein in an acceptable range, or, for lyophilized formulations described herein, provide an acceptable solution pH prior to lyophilization and/or after reconstitution.

Buffers that are useful in the pharmaceutical formulations and methods of the invention include succinate (sodium or potassium), L-histidine, phosphate (sodium or potassium), Tris (tris (hydroxymethyl) aminomethane), diethanolamine, citrate (sodium), acetate (sodium) and the like. In an embodiment of the invention, buffer is present in the formulation at a concentration of about 1-20 mM (1, 2, 3, 4, 5, 6, 7, 8, 9, 10, 11, 12, 13, 14, 15, 16, 17, 18, 19 and 20 mM). In specific embodiments of the invention, the buffer is histidine buffer. In another embodiment, the buffer is L-histidine buffer.

In one embodiment, the buffer has a pH in the range from about 4.5 to about 6.5. In another embodiment, the pH is in the range from about 5.0-6.0. In a further embodiment, the pH range is from about 5.3-5.8. In another embodiment, the pH is about 5.5. In arriving at the exemplary formulation, histidine and acetate buffers in the pH range of 5.0-6.0 were explored for suitability. When a range of pH values is recited, such as “a pH between pH 5.5 and 6.0,” the range is intended to be inclusive of the recited values. For example, a range from about 5.0 to about 6.0 includes 5.0, 5.1, 5.2, 5.3, 5.4, 5.5, 5.6, 5.7, 5.8, 5.9, and 6.0. For lyophilized formulations, unless otherwise indicated, the pH refers to the pH after reconstitution. pH is typically measured at 25° C. using standard glass bulb pH meter. As used herein, a solution comprising “histidine buffer at pH X” refers to a solution at pH X and comprising the histidine buffer, i.e., the pH is intended to refer to the pH of the solution. In some embodiments of the co-formulation in which the co-formulation contains a higher concentration of anti-human PD-1 antibody as compared to anti-CTLA4 antibody, the pH of the co-formulation is about 5.0.

In an embodiment of the invention, the anti-CTLA4 formulation and the co-formulation of anti-CTLA4 and anti-human PD-1 comprises a non-reducing sugar. As used herein, “non-reducing sugar” is a sugar not capable of acting as a reducing agent because it does not contain or cannot be converted to contain a free aldehyde group or a free ketone group. Examples of non-reducing sugars include but are not limited to disaccharides such as sucrose and trehalose. In an embodiment, the non-reducing sugar is present in an amount of from about 1-10% (w/v) (1, 2, 3, 4, 5, 6, 7, 8, 9 or 10%). In another embodiment, the non-reducing sugar is present in an amount from about 6% to about 8% (w/v) (6, 7, or 8%). In a further embodiment, the non-reducing sugar is present in an amount of about 6% (w/v). In a further embodiment, the non-reducing sugar is present in an amount of about 7% (w/v). In a further embodiment, the non-reducing sugar is present in an amount of about 8% (w/v). In one embodiment, the non-reducing sugar sucrose, trehalose, or raffinose. In another embodiment, the non-reducing sugar is sucrose. In a further embodiment, the sucrose is present at 6-8% w/v. In one embodiment, the sucrose is present at 6% (w/v). In one embodiment, the sucrose is present at 7% (w/v). In one embodiment, the sucrose is present at 8% (w/v).

The formulations described herein also comprise a surfactant. As used herein, a surfactant is a surface active agent that is amphipathic in nature. Surfactants may be added to the formulations herein to provide stability, reduce and/or prevent aggregation or to prevent and/or inhibit protein damage during processing conditions such as purification, filtration, freeze-drying, transportation, storage, and delivery. In one aspect of the invention, a surfactant may be useful for providing additional stability to the active ingredient(s).

Non-ionic surfactants that may be useful in the formulations and co-formulations described herein include, but are not limited to, polyoxyethylene sorbitan fatty acid esters (Polysorbates, sold under the trade name Tween® (Uniquema Americas LLC, Wilmington, DE)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate); polyoxyethylene alkyl ethers such as Brij® 58 (Uniquema Americas LLC, Wilmington, DE) and Brij® 35; poloxamers (e.g., poloxamer 188); Triton® X-100 (Union Carbide Corp., Houston, TX) and Triton® X-114; NP40; Span 20, Span 40, Span 60, Span 65, Span 80 and Span 85; copolymers of ethylene and propylene glycol (e.g., the Pluronic® series of nonionic surfactants such as Pluronic® F68, Pluronic® 10R5, Pluronic® F108, Pluronic® F127, Pluronic® F38, Pluronic® L44, Pluronic® L62 (BASF Corp., Ludwigshafen, Germany); and sodium dodecyl sulfate (SDS). In one embodiment, the non-ionic surfactant is polysorbate 80 or polysorbate 20. In one embodiment, the non-ionic surfactant is polysorbate 20. In another embodiment, the non-ionic surfactant is polysorbate 80.

The amount of non-ionic surfactant to be included in the formulations of the invention is an amount sufficient to perform the desired function, i.e., a minimal amount necessary to stabilize the active pharmaceutical ingredient (i.e., the anti-CTLA4 antibody or antigen binding fragment thereof, or both the anti-CTLA4 antibody or antigen binding fragment thereof and the anti-human PD-1 antibody or antigen binding fragment thereof) in the formulation. All percentages listed for polysorbate 80 are % w/v. Typically, the surfactant is present in a concentration of from about 0.008% to about 0.1% w/v. In some embodiments of this aspect of the invention, the surfactant is present in the formulation in an amount from about 0.01% to about 0.1%; from about 0.01% to about 0.09%; from about 0.01% to about 0.08%; from about 0.01% to about 0.07%; from about 0.01% to about 0.06%; from about 0.01% to about 0.05%; from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03%. In specific embodiments, the surfactant is present in an amount of about 0.02%. In alternative embodiments, the surfactant is present in an amount of about 0.01%, about 0.015%, about 0.025%, about 0.03%, about 0.035%, or about 0.04%.

In exemplary embodiments of the invention, the surfactant is a nonionic surfactant selected from the group consisting of: Polysorbate 20 and Polysorbate 80. In preferred embodiments, the surfactant is Polysorbate 80.

In specific embodiments, the formulations, including the co-formulations, of the invention comprise about 0.01% to about 0.04% w/v polysorbate 80. In further embodiments, the formulations described herein comprise polysorbate 80 in an amount of about 0.008% w/v, about 0.01% w/v. In one embodiment, the amount of polysorbate 80 is about 0.015 w/v %. In another embodiment, the amount of polysorbate 80 is about 0.02% w/v. In a further embodiment, the amount of polysorbate 80 is about 0.025% w/v. In another embodiment, the amount of polysorbate 80 is about 0.03% w/v. In a further embodiment, the amount of polysorbate 80 is about 0.035% w/v. In another embodiment, the amount of polysorbate 80 is about 0.04% w/v. In a further embodiment, the amount of polysorbate 80 is about 0.045% w/v. In particular embodiments, the formulations of the invention comprise about 0.02% w/v polysorbate 80.

The formulations, including the co-formulations, of the present invention also comprise methionine, or a pharmaceutically acceptable salt thereof. In one embodiment, the methionine is L-methionine. In another embodiment, the methionine is a pharmaceutically acceptable salt of L-methionine, such as, for example, methionine HCl. In an embodiment, methionine is present in the formulation at a concentration of about 1-20 mM (1, 2, 3, 4, 5, 6, 7, 8, 9, 10, 11, 12, 13, 14, 15, 16, 17, 18, 19 and 20 mM). In another embodiment, the methionine is present from about 5 mM to about 10 mM (5, 6, 7, 8, 9 and 10 mM). In another embodiment, the methionine is present at about 10 mM.

The formulations and co-formulations described herein may further comprise a chelating agent. In an embodiment of the invention, chelating agent is present in the formulation at a concentration of about 1-50 μM (e.g., 1, 5, 10, 15, 20, 25, 30, 35, 40, 45, or 50 μM). In one embodiment, the chelating agent is DTPA. In another embodiment, the chelating agent is EDTA. In some additional embodiment, the DTPA is the antioxidant which can be present in any of the following amounts 1, 5, 10, 15, 20, 25, 30, 35, 40, 45, or 50 μM in any of the formulations described herein.

### Lyophilized Compositions

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e., in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP, in an exemplary embodiment humanized anti-PD-1 antibody pembrolizumab, or antigen binding fragment thereof), i.e., in anticipation of reconstitution in a low volume of water. Subsequent dilution with water or isotonic buffer can then readily be used to dilute the DP to a lower concentration. Typically, excipients are included in a lyophilized formulation of the present invention at levels that will result in a roughly isotonic formulation when reconstituted at high DP concentration, e.g., for subcutaneous administration. Reconstitution in a larger volume of water to give a lower DP concentration will necessarily reduce the tonicity of the reconstituted solution, but such reduction may be of little significance in non-subcutaneous, e.g., intravenous, administration. If isotonicity is desired at lower DP concentration, the lyophilized powder may be reconstituted in the standard low volume of water and then further diluted with isotonic diluent, such as 0.9% sodium chloride.

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about-30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g., 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the present invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. In one embodiment, the protein concentration after reconstitution is about 10-300 mg/ml. In one embodiment, the protein concentration after reconstitution is about 20-250 mg/ml. In one embodiment, the protein concentration after reconstitution is about 150-250 mg/ml. In one embodiment, the protein concentration after reconstitution is about 180-220 mg/ml. In one embodiment, the protein concentration after reconstitution is about 50-150 mg/ml. In one embodiment, the protein concentration after reconstitution is about 100 mg/ml. In one embodiment, the protein concentration after reconstitution is about 75 mg/ml. In one embodiment, the protein concentration after reconstitution is about 50 mg/ml. In one embodiment, the protein concentration after reconstitution is about 25 mg/ml. High protein concentrations are particularly useful where subcutaneous delivery of the reconstituted formulation is intended. However, for other routes of administration, such as intravenous administration, lower concentrations of the protein may be desired (e.g., from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g., phosphate-buffered saline), sterile saline solution, Ringer's solution or dextrose solution.

### Liquid Compositions

A liquid antibody formulation can be made by taking the drug substance (e.g., anti-humanized PD-1) which is in liquid form (e.g., pembrolizumab in an aqueous formulation) and buffer exchanging it into the desired buffer as the last step of the purification process. There is no lyophilization step in this embodiment. The drug substance in the final buffer is concentrated to a desired concentration. Excipients such as sucrose and polysorbate 80 are added to the drug substance and it is diluted using the appropriate buffer to final protein concentration. The final formulated drug substance is filtered using 0.22 μm filters and filled into a final container (e.g., glass vials).

### III. Methods of Use

The invention also relates to a method of treating cancer in a subject, the method comprising administering an effective amount of any of the formulations of the invention; i.e., any formulation described herein, to the subject. In some specific embodiments of this method, the formulation is administered to the subject via intravenous administration. In other embodiments, the formulation is administered to the subject by subcutaneous administration. In one embodiment, the invention comprises a method of treating cancer in a human patient comprising administering any formulation of the invention to the patient.

In any of the methods of the invention, the cancer can be selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g., hormone refractory prostate adenocarcinoma), pancreatic cancer, colon cancer, esophageal cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma.

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In one embodiment, the invention comprises a method of treating metastatic non-small cell lung cancer (NSCLC) in a human patient comprising administering a formulation of the invention to the patient. In specific embodiments, the patient has a tumor with high PD-L1 expression [(Tumor Proportion Score (TPS)≥50%)] and was not previously treated with platinum-containing chemotherapy. In other embodiments, the patient has a tumor with PD-L1 expression (TPS≥1%) and was previously treated with platinum-containing chemotherapy. In still other embodiments, the patient has a tumor with PD-L1 expression (TPS≥1%) and was not previously treated with platinum-containing chemotherapy. In specific embodiments, the patient had disease progression on or after receiving platinum-containing chemotherapy. In certain embodiments, the PD-L1 TPS is determined by an FDA-approved test. In certain embodiments, the patient's tumor has no EGFR or ALK genomic aberrations. In certain embodiments, the patient's tumor has an EGFR or ALK genomic aberration and had disease progression on or after receiving treatment for the EGFR or ALK aberration(s) prior to receiving the anti-PD-1 antibody, or antigen binding fragment thereof.

In some embodiments, the cancer is metastatic colorectal cancer with high levels of microsatellite instability (MSI-H).

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

Additional cancers that can benefit from treatment with the formulations described herein include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

The formulations can also be used to prevent or treat infections and infectious disease. Thus, the invention provides a method for treating chronic infection in a mammalian subject comprising administering an effective amount of a formulation of the invention to the subject. In some specific embodiments of this method, the formulation is administered to the subject via intravenous administration. In other embodiments, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist.

Suitable routes of administration may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g., subcutaneous administration) require a lyophilized formulation to enable reconstitution at high concentration.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g., biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

Embodiments of the invention also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of and CTLA4 or PD-1-related disease; (g) halting or delaying the progression cancer; (h) stabilization of CTLA4 or PD-1-related disease; (i) inhibiting the growth or survival of tumor cells; (j) eliminating or reducing the size of one or more cancerous lesions or tumors; (k) reduction of the progression, onset or severity of CTLA4 or PD-1-related disease; (1) reducing the severity or duration of the clinical symptoms of CTLA4 or PD-1-related disease such as cancer (m) prolonging the survival of a patient relative to the expected survival in a similar untreated patient n) inducing complete or partial remission of a cancerous condition or other CTLA4 or PD-1 related disease, o) treatment of cancer; or p) treatment of chronic infections.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

### Example 1

Anti-CTLA4 Antibody Formulation Stability with or without Methionine

This study was conducted to study the effect of 10 mM L-Methionine on stability of an anti-CTLA4 antibody formulation. The effects of the following stresses were evaluated on anti-CTLA4 formulations with and without L-Methionine:
(1) Thermal stress at 5±3° C. (ambient humidity), 25° C. (60% relative humidity), 40° C. (75% relative humidity)—up to 3 months.
(2) Agitation stress in a horizontal position (300 rpm for 3 days)
(3) Freeze-thaw stress (five freeze-thaw cycles at −80° C. to 18-22° C. (room temperature for a 4 hour thaw)).
(4) Light stress (ICH conditions under 0.2×ICH, 0.5×ICH, 1×ICH).

Based on the data, a formulation containing L-methionine is more stable than a corresponding formulation without L-methionine.

### Materials and Methods

The following liquid formulations were prepared using an anti-CTLA4 antibody having the followings CDRs: CDRH1 of SEQ ID NO:35, CDRH2 of SEQ ID NO: 36, CDRH3 of SEQ ID NO: 37, CDRL1 of SEQ ID NO: 38, CDRL2 of SEQ ID NO: 39, and CDRL3 of SEQ ID NO: 40 on an IgG1 backbone. The variable heavy chain and variable light chain sequences for the anti-CTLA4 antibody are set forth in SEQ ID NO: 88 and 48, respectively. Each formulation was filled at 1 mL into 2-mL Type-1 glass vials. A total of 36 vials for formulation A1 and 19 vials for formulations A2 were filled. The target pH for each formulation was 5.5.

TABLE 10
anti-CTLA4 antibody formulations

| Formulation | | | | | |
|---|---|---|---|---|---|
| Number | Description | | | | |
| A1 | Anti-CTLA4 | 10 mM L- | 7% | 0.02% | 10 mM |
| | antibody (50 | Histidine | Sucrose | PS80 | L-Met |
| | mg/mL) | buffer | (w/v) | (w/v) | |
| A2 | Anti-CTLA4 | 10 mM L- | 7% | 0.02% | NA |
| | antibody (50 | Histidine | Sucrose | PS80 | |
| | mg/mL) | buffer | (w/v) | (w/v) | |

The vials were then incubated at three different storage conditions: 5° C. (ambient humidity), 25° C. (60% relative humidity), and 40° C. (75% relative humidity). Data was collected as follows:

Photostability studies were conducted using 1 mL liquid formulations of A1 and A2 in glass vials at room temperature under 0.2×ICH; 0.5×ICH; and 1×ICH.

Protein concentrations were measured by using UV absorbance at 280 nm.

Samples were equilibrated to room temperature and turbidity studies (A350) were conducted on the samples at spectrophotometric absorbance at 350 nm.

Samples were assessed by size exclusion chromatography (SEC) for purity in which the percentage of monomer was determined, as well as the percentages of high molecular weight species (HMW) and late eluting peaks (LMW species). Ultra Performance-Size Exclusion Chromatography (UP-SEC) was performed by diluting the samples to 5.0 mg/mL in mobile phase (50 mM sodium phosphate, 450 mM arginine monohydrochloride, pH 7.0). The diluted samples were injected (6 μL) into a UPLC equipped with a Waters BEH200 column and a UV detector. Proteins in the sample were separated by size and detected by UV absorption at 280 nm.

Ion exchange chromatography was performed to evaluate the chemical stability and to monitor the change in the charge variant profile over time. An ion exchange HPLC method was performed using a Dionex ProPac WCX-10 column and a UV detector at 280 nm. Samples were diluted in purified water, and 80 μg were injected for analysis. The mobile phase used for the IEX analysis of the thermal stability samples was a gradient of the following mobile phases (mobile phase A: 20 mM MOPS, pH 7.2; mobile phase B: 50 mM sodium phosphate, 60 mM sodium chloride pH 8.0). The assay is performed using a mobile phase gradient from 20 mM MOPS, pH 7.2 to 50 mM sodium phosphate, 60 mM NaCl, pH 8.0. UV detection is performed at 280 nm. These methods are considered equivalent, and results are presented as relative percentages based on the total area of the chromatogram.

Peptide mapping was performed by Lys-C digestion. Samples were injected on Q Exactive at 30 μl/sample. The data analysis was done by PinPoint software.

The results of the studies are set forth in the tables below:

TABLE 11

| | Storage Condition | | | |
|---|---|---|---|---|
| | 5° C. | | | |
| | Formulations | | | |
| | Formulation A1 | | | |
| | (with L-Methionine) | | | |
| | Time (weeks) | | | |
| | 0 | 4 | 8 | 12 |
| pH | 5.7 | 5.7 | 5.7 | NT |
| Concentration (mg/mL) | 54.8 | 54.1 | 53.7 | NT |
| (A₂₈₀ nm) | | | | |
| Turbidity (A₃₅₀ nm) | 0.135 | 0.135 | 0.133 | NT |
| UPSEC | | | | |
| High Molecular | 1.07 | 1.11 | 1.13 | 1.16 |
| Weight Species (%) | | | | |
| Low Molecular | 0.06 | 0.10 | 0.07 | 0.01 |
| Weight Peaks (%) | | | | |
| Monomer (%) | 98.9 | 98.8 | 98.8 | 98.8 |
| HP-IEX | | | | |
| Acidic variants (%) | 21.79 | 21.85 | 23.08 | 22.88 |
| Basic Variants (%) | 10.79 | 9.89 | 9.94 | 10.60 |
| Main (%) | 67.4 | 68.3 | 67.0 | 66.5 |
| Peptide Mapping | | | | |
| % LC-M4 oxidation | 0.1 | NT | NT | NT |
| % HC-M34 oxidation | 0.3 | NT | NT | NT |
| % HC-M250 oxidation | 2.3 | NT | NT | NT |
| % HC-M426 oxidation | 0.8 | NT | NT | NT |

TABLE 12

| | Storage Condition | | | |
|---|---|---|---|---|
| | 5° C. | | | |
| | Formulations | | | |
| | Formulation A2 | | | |
| | Without L-methionine | | | |
| | Time (weeks) | | | |
| | 0 | 4 | 8 | 12 |
| pH | 5.7 | 5.7 | 5.7 | NT |
| Concentration (mg/mL) | 53.6 | 54.3 | 54.0 | NT |
| (A₂₈₀ nm) | | | | |
| Turbidity (A₃₅₀ nm) | 0.132 | 0.135 | 0.132 | NT |
| UPSEC | | | | |
| High Molecular | 1.06 | 1.13 | 1.8 | NT |
| Weight Species (%) | | | | |
| Low Molecular | 0.05 | 0.06 | 0.07 | NT |
| Weight Peaks (%) | | | | |
| Monomer (%) | 98.9 | 98.8 | 98.8 | NT |
| HP-IEX | | | | |
| Acidic variants (%) | 21.94 | 23.20 | 22.97 | NT |
| Basic Variants (%) | 8.67 | 8.94 | 11.95 | NT |
| Main (%) | 69.4 | 67.8 | 65.1 | NT |
| Peptide Mapping | | | | |
| % LC-M4 oxidation | 0.1 | NT | NT | NT |
| % HC-M34 oxidation | 0.3 | NT | NT | NT |
| % HC-M250 oxidation | 2.3 | NT | NT | NT |
| % HC-M426 oxidation | 1.1 | NT | NT | NT |

TABLE 13

| | Storage Condition | | | |
|---|---|---|---|---|
| | 25° C. | | | |
| | Formulations | | | |
| | Formulation 1 | | | |
| | With L-Methionine | | | |
| | Time (weeks) | | | |
| | 0 | 4 | 8 | 12 |
| pH | 5.7 | 5.7 | 5.7 | NT |
| Concentration (mg/mL) | 54.8 | 54.6 | 53.8 | NT |
| (A₂₈₀ nm) | | | | |
| Turbidity (A₃₅₀ nm) | 0.135 | 0.139 | 0.143 | NT |
| UPSEC | | | | |
| High Molecular | 1.07 | 1.20 | 1.24 | 1.22 |
| Weight Species (%) | | | | |
| Low Molecular | 0.06 | 0.14 | 0.23 | 0.18 |
| Weight Peaks (%) | | | | |
| Monomer (%) | 98.9 | 98.7 | 98.5 | 98.6 |
| HP-IEX | | | | |
| Acidic variants (%) | 21.79 | 23.48 | 26.53 | 28.85 |
| Basic Variants (%) | 10.79 | 8.95 | 11.94 | 11.79 |
| Main (%) | 67.4 | 67.6 | 61.5 | 59.4 |
| Peptide Mapping | | | | |
| % LC-M4 oxidation | 0.1 | NT | NT | NT |
| % HC-M34 oxidation | 0.3 | NT | NT | NT |
| % HC-M250 oxidation | 2.3 | NT | NT | NT |
| % HC-M426 oxidation | 0.8 | NT | NT | NT |

TABLE 14

| | Storage Condition | | | |
|---|---|---|---|---|
| | 25° C. | | | |
| | Formulations | | | |
| | Formulation A2 | | | |
| | Without L-Methionine | | | |
| | Time (weeks) | | | |
| | 0 | 4 | 8 | 12 |
| pH | 5.7 | 5.8 | 5.8 | NT |
| Concentration (mg/mL) | 53.6 | 54.8 | 54.2 | NT |
| (A₂₈₀ nm) | | | | |
| Turbidity (A₃₅₀ nm) | 0.132 | 0.142 | 0.142 | NT |
| UPSEC | | | | |
| High Molecular | 1.06 | 1.24 | 1.33 | 1.30 |
| Weight Species (%) | | | | |
| Low Molecular | 0.05 | 0.14 | 0.25 | 0.17 |
| Weight Peaks (%) | | | | |
| Monomer (%) | 98.9 | 98.6 | 98.4 | 98.5 |
| HP-IEX | | | | |
| Acidic variants (%) | 21.94 | 25.28 | 26.91 | 29.69 |
| Basic Variants (%) | 8.67 | 10.97 | 11.91 | 12.01 |
| Main (%) | 69.4 | 63.8 | 61.2 | 58.3 |
| Peptide Mapping | | | | |
| % LC-M4 oxidation | 0.1 | NT | NT | NT |
| % HC-M34 oxidation | 0.3 | NT | NT | NT |
| % HC-M250 oxidation | 2.3 | NT | NT | NT |
| % HC-M426 oxidation | 1.1 | NT | NT | NT |

TABLE 15

| | Storage Condition | | | |
|---|---|---|---|---|
| | 40° C. | | | |
| | Formulations | | | |
| | Formulation A1 | | | |
| | With L-Methionine | | | |
| | Time (weeks) | | | |
| | 0 | 4 | 8 | 12 |
| pH | 5.7 | 5.7 | 5.7 | NT |
| Concentration (mg/mL) | 54.8 | 54.8 | 54.2 | NT |
| (A₂₈₀ nm) | | | | |
| Turbidity (A₃₅₀ nm) | 0.135 | 0.156 | 0.183 | NT |
| UPSEC | | | | |
| High Molecular | 1.07 | 1.22 | 1.29 | 1.64 |
| Weight Species (%) | | | | |
| Low Molecular | 0.06 | 0.53 | 0.96 | 2.32 |
| Weight Peaks (%) | | | | |
| Monomer (%) | 98.9 | 98.2 | 97.7 | 96.0 |
| HP-IEX | | | | |
| Acidic variants (%) | 21.79 | 36.96 | 49.35 | 61.09 |
| Basic Variants (%) | 10.79 | 11.74 | 13.82 | 12.51 |
| Main (%) | 67.4 | 51.3 | 36.8 | 26.4 |
| Peptide Mapping | | | | |
| % LC-M4 oxidation | 0.1 | NT | 0.2 | NT |
| % HC-M34 oxidation | 0.3 | NT | 0.4 | NT |
| % HC-M250 oxidation | 2.3 | NT | 2.6 | NT |
| % HC-M426 oxidation | 0.8 | NT | 1.0 | NT |

TABLE 16

| | Storage Condition | | | |
|---|---|---|---|---|
| | 40° C. | | | |
| | Formulations | | | |
| | Formulation A2 | | | |
| | Without L-Methionine | | | |
| | Time (weeks) | | | |
| | 0 | 4 | 8 | 12 |
| pH | 5.7 | 5.8 | 5.8 | NT |
| Concentration (mg/mL) | 53.6 | 54.9 | 54.2 | NT |
| (A₂₈₀ nm) | | | | |
| Turbidity (A₃₅₀ nm) | 0.132 | 0.168 | 0.212 | NT |
| UPSEC | | | | |
| High Molecular | 1.06 | 1.37 | 1.54 | 1.62 |
| Weight Species (%) | | | | |
| Low Molecular | 0.05 | 0.58 | 1.04 | 2.27 |
| Weight Peaks (%) | | | | |
| Monomer (%) | 98.9 | 98.1 | 97.5 | 96.1 |
| HP-IEX | | | | |
| Acidic variants (%) | 21.94 | 38.15 | 51.39 | 62.84 |
| Basic Variants (%) | 8.67 | 14.43 | 12.87 | 12.44 |
| Main (%) | 69.4 | 47.4 | 35.7 | 24.7 |
| Peptide Mapping | | | | |
| % LC-M4 oxidation | 0.1 | NT | 0.2 | NT |
| % HC-M34 oxidation | 0.3 | NT | 0.3 | NT |
| % HC-M250 oxidation | 2.3 | NT | 5.0 | NT |
| % HC-M426 oxidation | 1.1 | NT | 2.0 | NT |

TABLE 17

| | Formulation A1 | | | | | | |
|---|---|---|---|---|---|---|---|
| Formulation | With L-Methionine | | | | | | |
| | | | Agitation¹ | | Photostability (ICH) | | |
| Stress | T0 | Freeze-Thaw | 1 d | 3 d | 0.2X | 0.5X | 1X |
| pH | 5.7 | 5.8 | 5.8 | 5.8 | 5.7 | 5.7 | 5.7 |
| Concentration (mg/mL) | 54.8 | 54.8 | 54.3 | 53.2 | 54.5 | 55.2 | 53.8 |
| (A₂₈₀ nm) | | | | | | | |
| Turbidity (A₃₅₀ nm) | 0.135 | 0.133 | 0.132 | 0.133 | 0.181 | 0.248 | 0.412 |

UPSEC

| | | | | | | | |
|---|---|---|---|---|---|---|---|
| High Molecular Weight | 1.07 | 1.10 | 1.08 | 1.11 | 1.58 | 2.13 | 3.06 |
| Species (%) | | | | | | | |
| Low Molecular Weight | 0.06 | 0.06 | 0.05 | 0.06 | 0.16 | 0.26 | 0.45 |
| Peaks (%) | | | | | | | |
| Monomer (%) | 98.9 | 98.8 | 98.9 | 98.8 | 98.3 | 97.6 | 96.5 |

HP-IEX

| | | | | | | | |
|---|---|---|---|---|---|---|---|
| Acidic variants (%) | 21.79 | 22.65 | 23.08 | 22.73 | 26.19 | 25.89 | 37.26 |
| Basic Variants (%) | 10.79 | 11.50 | 10.84 | 11.15 | 20.75 | 20.48 | 30.92 |
| Main (%) | 67.4 | 65.9 | 66.1 | 66.1 | 53.1 | 53.6 | 31.8 |

Peptide Mapping

| | | | | | | | |
|---|---|---|---|---|---|---|---|
| % LC-M4 oxidation | 0.1 | NT | NT | NT | 0.3 | 0.4 | 0.8 |
| % HC-M34 oxidation | 0.3 | NT | NT | NT | 0.4 | 0.4 | 0.4 |
| % HC-M250 oxidation | 2.3 | NT | NT | NT | 10.7 | 18.8 | 31.6 |
| % HC-M426 oxidation | 0.8 | NT | NT | NT | 7.2 | 13.7 | 25.8 |

TABLE 18

| | Formulation A1 | | | | | | |
|---|---|---|---|---|---|---|---|
| Formulation | With L-Methionine | | | | | | |
| | | | Agitation¹ | | Photostability (ICH) | | |
| Stress | T0 | Freeze-Thaw | 1 d | 3 d | 0.2X | 0.5X | 1X |
| pH | 5.7 | NT | NT | NT | 5.7 | 5.7 | 5.6 |
| Concentration (mg/mL) | 53.6 | NT | NT | NT | 53.9 | 53.8 | 54.3 |
| (A₂₈₀ nm) | | | | | | | |
| Turbidity (A₃₅₀ nm) | 0.132 | NT | NT | NT | 0.187 | 0.272 | 0.466 |

UPSEC

| | | | | | | | |
|---|---|---|---|---|---|---|---|
| High Molecular Weight | 1.06 | NT | NT | NT | 1.77 | 2.48 | 3.72 |
| Species (%) | | | | | | | |
| Low Molecular Weight | 0.05 | NT | NT | NT | 0.16 | 0.28 | 0.61 |
| Peaks (%) | | | | | | | |
| Monomer (%) | 98.9 | NT | NT | NT | 98.1 | 97.3 | 95.9 |

HP-IEX

| | | | | | | | |
|---|---|---|---|---|---|---|---|
| Acidic variants (%) | 21.94 | NT | NT | NT | 27.81 | 32.36 | 38.17 |
| Basic Variants (%) | 8.67 | NT | NT | NT | 22.05 | 29.06 | 34.19 |
| Main (%) | 69.4 | NT | NT | NT | 50.1 | 38.6 | 27.6 |

Peptide Mapping

| | | | | | | | |
|---|---|---|---|---|---|---|---|
| % LC-M4 oxidation | 0.1 | NT | NT | NT | 0.3 | 0.4 | 0.7 |
| % HC-M34 oxidation | 0.3 | NT | NT | NT | 0.3 | 0.3 | 0.4 |
| % HC-M250 oxidation | 2.3 | NT | NT | NT | 13.3 | 24.9 | 43.2 |
| % HC-M426 oxidation | 1.1 | NT | NT | NT | 9.0 | 18.9 | 33.7 |

### Results

There were no measurable changes in protein concentration between the formulations for the conditions and duration of the study, as measured by UV absorbance at 280 nm.

There were no measurable changes in the pH between the formulations for the conditions tested and the duration of the study.

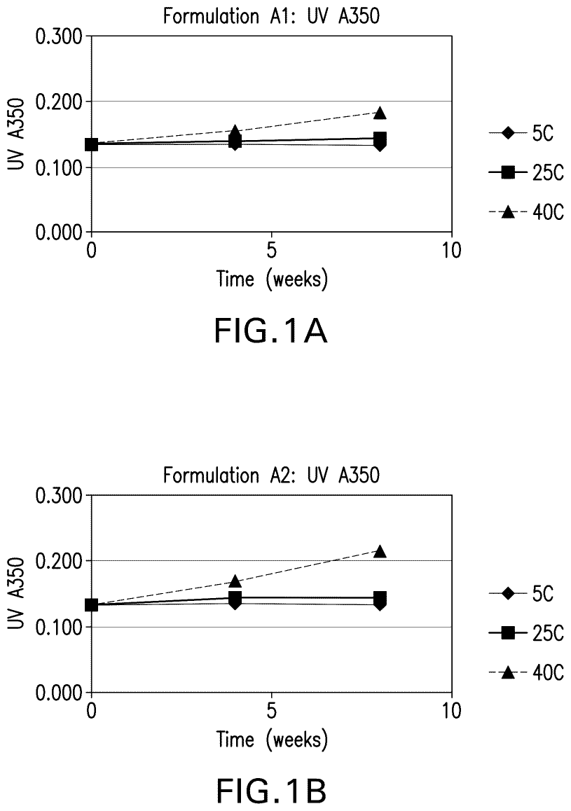

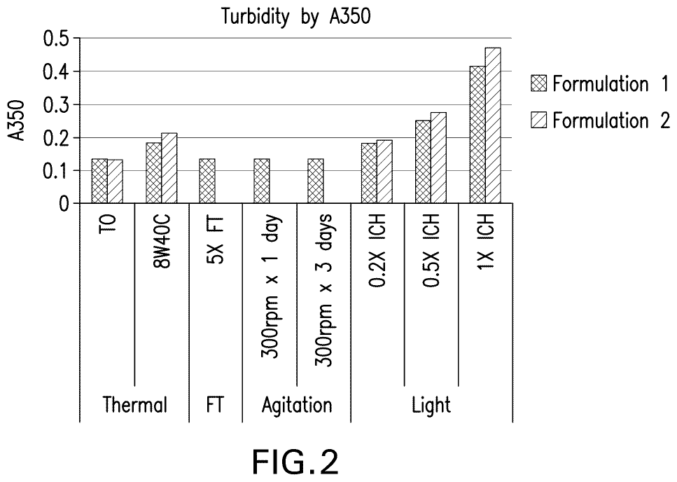

Turbidity (A350) data is shown in FIG. 1A and FIG. 1B. At 40° C., both the formulations showed a trend of increase in turbidity for up to 8-week time point. For both formulations, there was no substantial changes in turbidity for up to 8-week time point at 25° C. and 5° C. As shown in FIG. 2, Formulation A2 exhibited a slightly more (but consistent) increase in turbidity for all light-stress conditions as compared to Formulation A1. Treatment of Formulation A1 samples to either freeze-thaw (up to 5×) or agitation stresses (300 rpm, for up to 3 days) did not change sample turbidity compared to control (TO) samples. Formulation A2 was not subjected to freeze-thaw or agitation stresses. Thus, Formulation A1, containing L-methionine is slightly preferable based on the turbidity data.

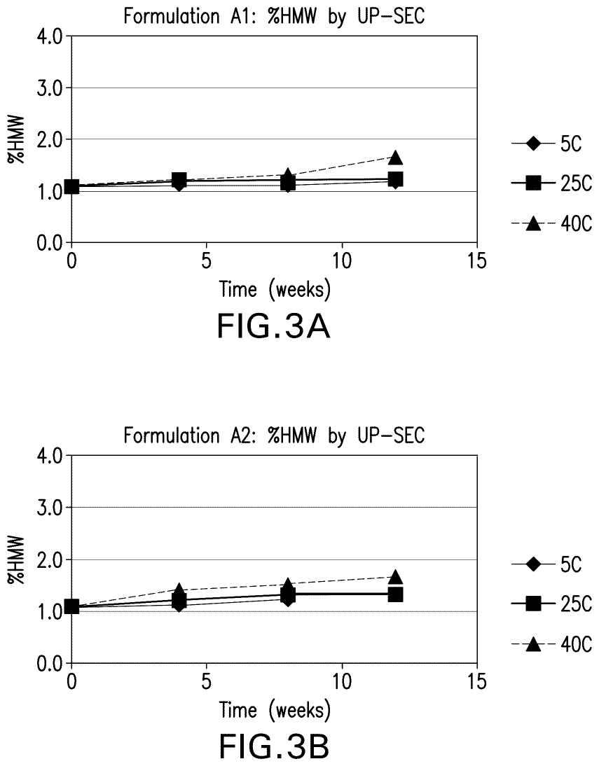

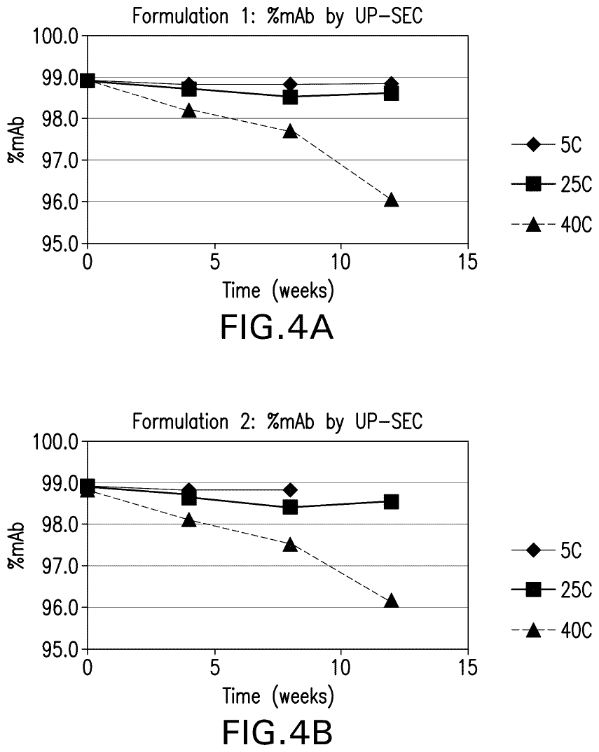

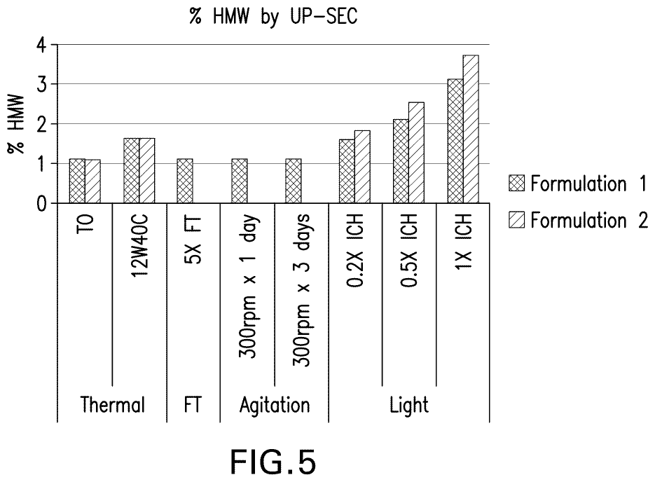

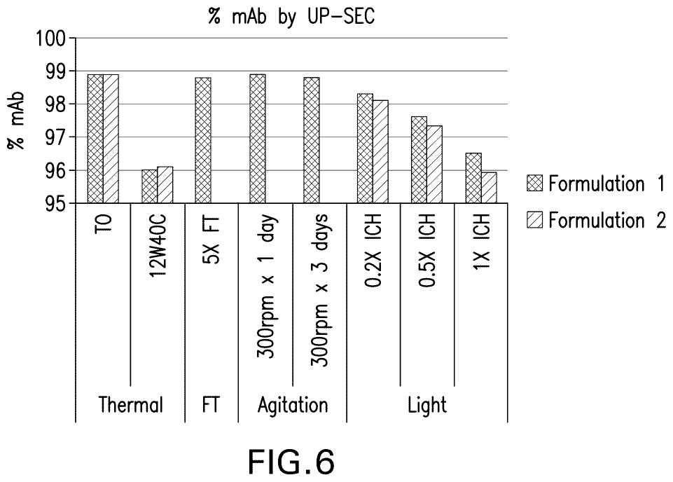

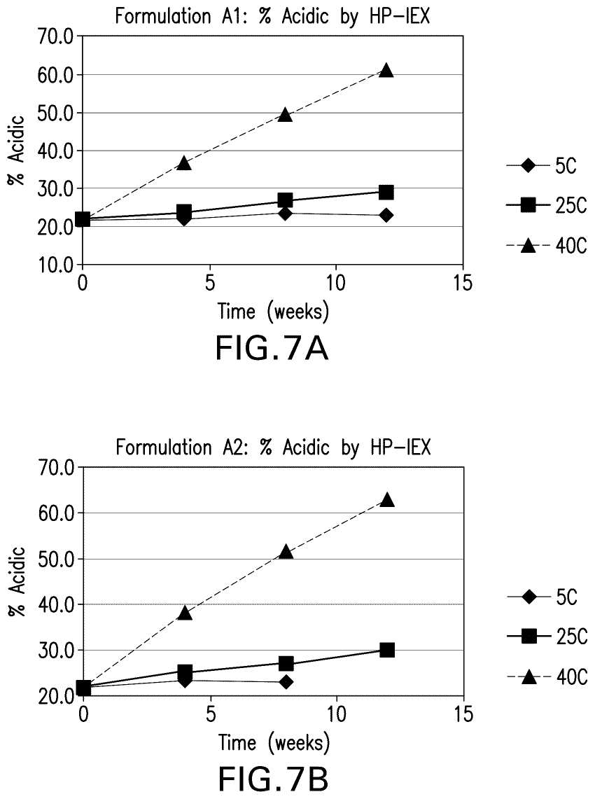

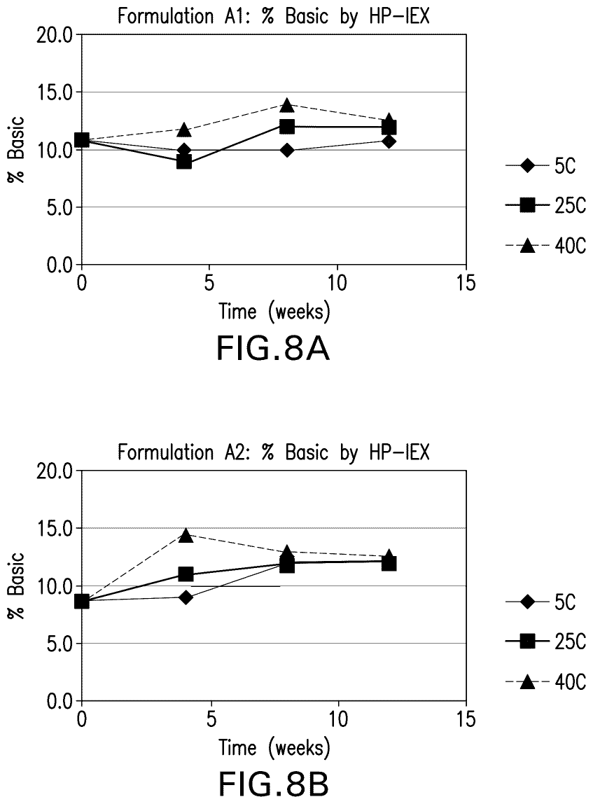

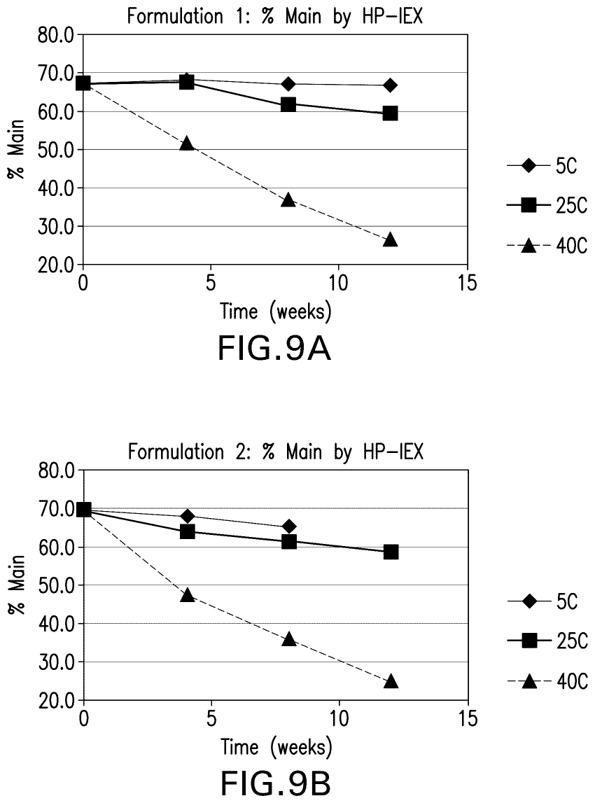

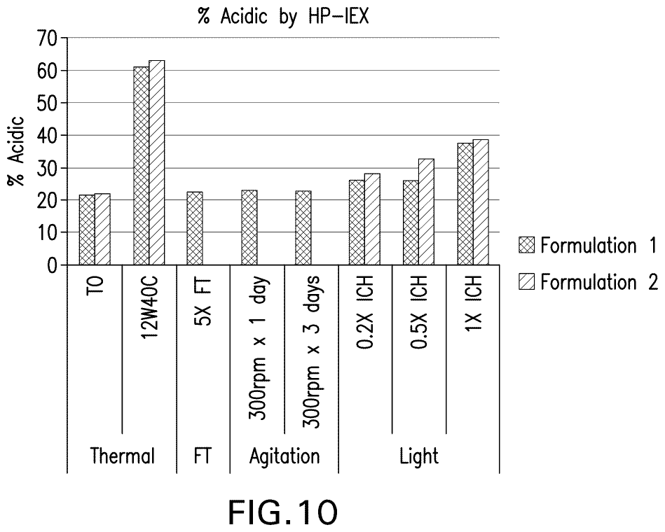

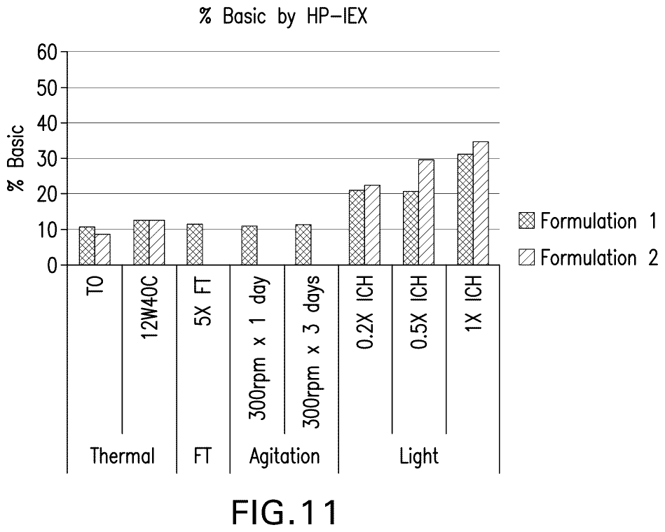

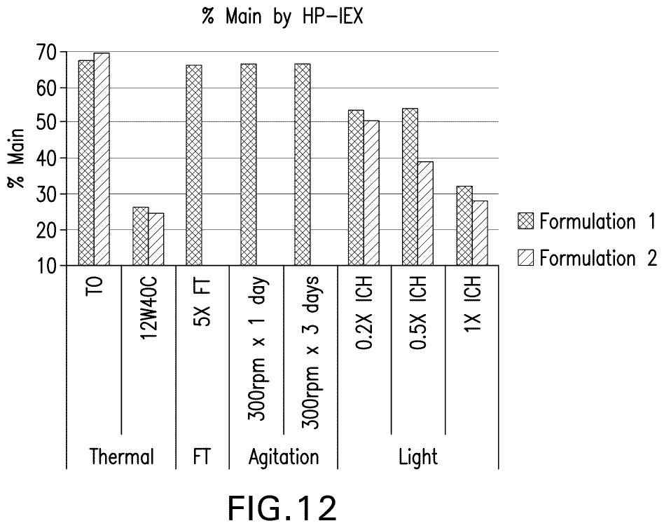

As shown in FIGS. 3A, 3B, 4A and 4B, UP-SEC analysis of the samples to determine the percentage of HMW and percentage of monomer indicated that at 40° C., both the formulations showed a trend of increase in % HMW peak and % LMW peak (and a consequent decrease in % monomer peak) for up to 12-week time point. At 25° C., both the formulations showed similar trends, but smaller changes, as compared to 40° C. At 5° C., no substantial changes were observed. As shown in FIG. 5, formulation A2 shows a slightly more, but consistent, increase in % HMW (and corresponding slightly more, but consistent, decrease in % monomer) for all light-stress conditions studied as compared to Formulation A1. Treatment of Formulation A1 samples to either freeze-thaw (up to 5×) or agitation stresses (300 rpm, for up to 3 days) did not change % HMW or % monomer compared to control (TO) samples (see FIG. 5 and FIG. 6). Formulation A2 was not subjected to freeze-thaw or agitation stresses. Thus, Formulation A1, containing L-methionine, is slightly preferable based on the UP-SEC data. As shown in FIGS. 7A, 7B, 8A, 8B, 9A and 9B, the HP-IEX data indicates that at 40° C., both the formulations showed a trend of increase in % acidic peak and % basic peak up to 12-week time point, along with a corresponding trend of decrease in the % main peak. At 25° C., both the formulations showed similar trends, but smaller changes, as compared to 40° C. At 5° C., no substantial changes were observed for Formulation 1 or Formulation 2, except for 8-week time point for Formulation A2, where a small increase in % basic peak and corresponding small decrease in % main peak was observed. This trend could not be confirmed at 12-week/5° C. time point since Formulation A2 samples were not tested. As shown in FIGS. 10-12, Formulation A2 shows a slightly more, but consistent, increase in % acidic and % basic peaks (and corresponding slightly more, but consistent, decrease in % main peak) for all light-stress conditions, along with a corresponding decrease in the % main peak (as compared to Formulation A1). Also shown in FIGS. 10-12, treatment of Formulation A1 samples to either freeze-thaw (up to 5×) or agitation stresses (300 rpm, for up to 3 days) did not change % acidic peak, % basic peak, or % main peak compared to control (TO) samples (Formulation 2 was not subjected to freeze-thaw or agitation stresses). Thus, Formulation A1, containing L-methionine, is slightly preferable based on the HP-IEX data.

Monoclonal antibodies frequently have methionine residues in the CDR region and the Fc region that may be liable for oxidation under light stress. For the anti-CTLA4 antibody, LC-M4, HC-M34, HC-M250 and HC-M426 could be liable for oxidation under light stress. Peptide mapping studies were performed to determine the changes in oxidation level of these residues upon 8 week exposure at 40° C. or 0.2×/0.5×/1×ICH light stress treatment.

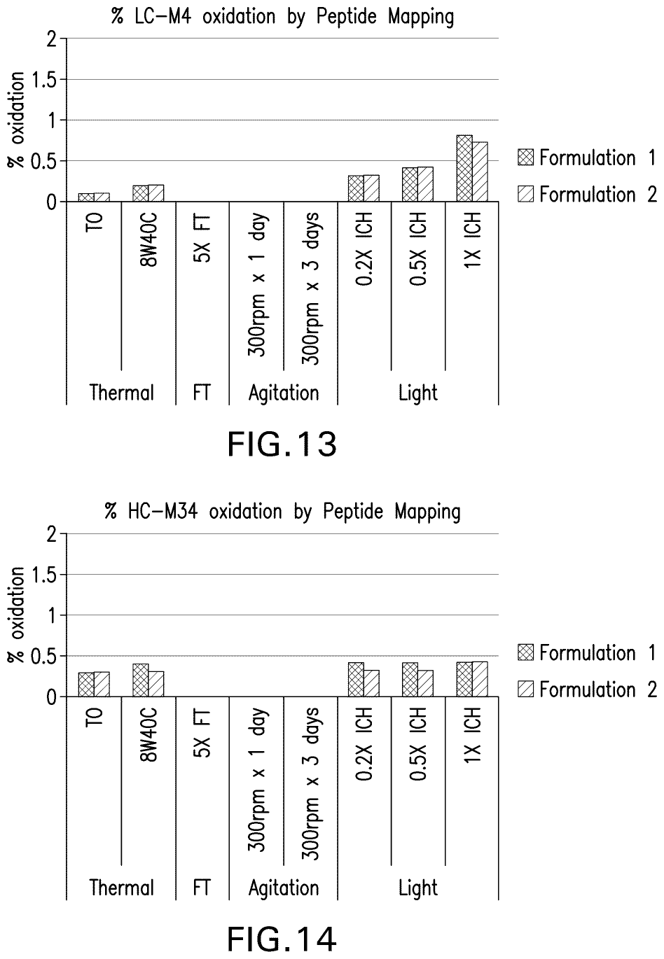

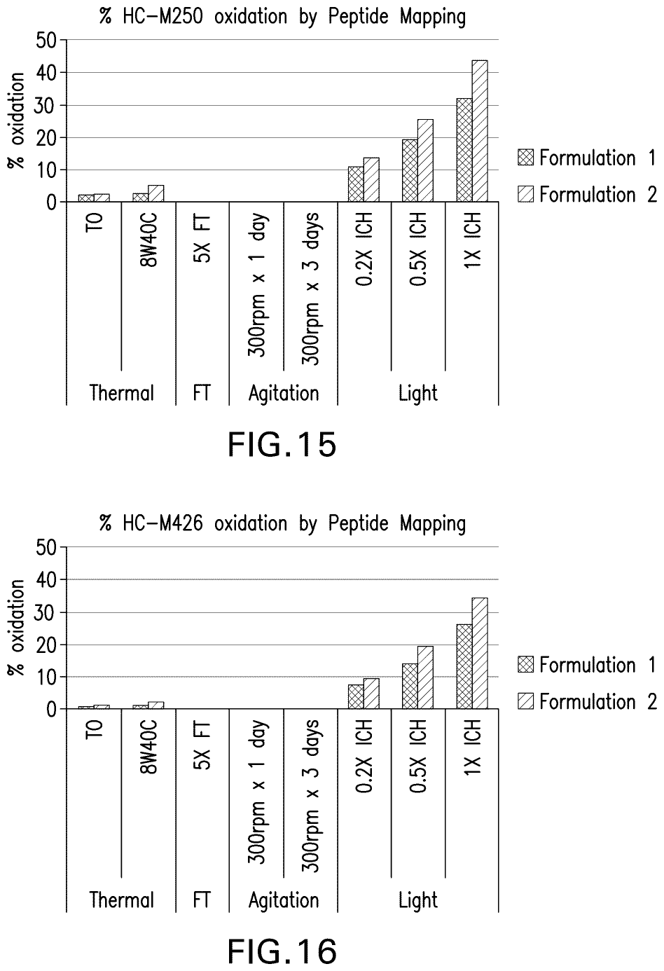

The results of the peptide mapping studies showing oxidation percent oxidation in residues LC-M4, HC-M34, HC-M250 and HC-M426 are represented in FIG. 13, FIG. 14, FIG. 15 and FIG. 16 respectively.

As showing in FIGS. 13-16, under light stress conditions there is an increased oxidation of certain methionine residues. Notably, residues HC-M250 and HC-M426 showed significant increase in oxidation levels upon 0.5× and 1×ICH light stress treatment in both formulations. However, presence of 10 mM L-methionine in Formulation A1 resulted in smaller increase in oxidation levels of M250 and M426 as compared to Formulation A2 which did not contain L-methionine. Thus, Formulation 1 (with L-Met) would seem slightly preferable based on the peptide-mapping data.

Based on a comparison of the analytical data from the above studies, formulation A1 as compared to formulation A1 exhibited (i) an increase in turbidity for all light-stress conditions, (ii) lower increase in aggregate levels (% HMW) for all light stress conditions, (iii) slightly less, but consistent, decrease in the % main peak for all light-stress conditions, and (iv) lower increase in oxidation levels of residues HC M250 and HC M426 following light stress.

### Example 2

### Anti-CTLA4 Antibody Formulation Buffer Screen

This study compares the stability of an anti-CLTA4 antibody comprising a heavy chain variable region comprising SEQ ID NO: 88 and a light chain variable region comprising SEQ ID NO: 48 in two different viable formulation buffers (L-histidine and acetate) in the presence of sucrose, polysorbate 80 and L-methionine. Protein-protein interactions (indicative of colloidal and thermal stability) of the two formulations were measured (in L-histidine and acetate buffers as shown below). A repulsive protein-protein interaction, as indicated by a positive diffusion interaction parameter (K_{D}) values (K_{D}>0), indicates a stable formulation with low propensity for aggregation. The K_{D} for both the formulations at three different pH (pH 5, 5.5 and 6) were measured at least three times each (N=3) to obtain a standard deviation. Based on the protein-protein interactions (data not shown or Fig Y), the anti-CTLA4 antibody is stable in both the L-histidine and acetate buffer across a pH range of 5.0-6.0. Hence, the two formulations were placed on additional thermal stability at 5° C., 25° C. and 40° C. at pH 5.5.

To evaluate the stability of the formulations, the effects of the following stresses were evaluated on the two anti-CTLA4 formulations (L-histidine buffer and acetate buffer):
(1) Thermal stress at 5±3° C. (ambient humidity), 25° C. (60% relative humidity), 40° C. (75% relative humidity)—up to 3 months.
(2) Agitation stress in a horizontal position (300 rpm for 3 days)
(3) Freeze-thaw stress (five freeze-thaw cycles at −80° C. to 18-22° C. (room temperature for a 4 hour thaw)).
(4) Light stress (ICH conditions under 0.2×ICH, 0.5×ICH, 1×ICH).

Based on the data, the anti-CTLA4 antibody is stable in both the L-histidine buffer and the acetate buffer.

### Materials and Methods

The following liquid formulations were prepared using an anti-CTLA4 antibody of having the followings CDRs: HCDR1 of SEQ ID NO:35, HCDR2 of SEQ ID NO: 36, HCDR3 of SEQ ID NO: 37, LCDR1 of SEQ ID NO: 38, LCDR2 of SEQ ID NO: 39, and LCDR3 of SEQ ID NO: 40 on an IgG1 backbone. Each formulation was filled at 1 mL into 2-mL Type-1 glass vials. A total of 72 batches each for the two formulations were manufactured. The table below lists the compositions of the two formulations. Sucrose 7% (w/v) is added as a stabilizer; PS-80 is a surfactant which imparts stability against agitation induced stress; and L-methionine is an anti-oxidant as it reduced methionine oxidation under light stress conditions (see Example 1 above).

TABLE 19
Formulation

| Formulation | | | | | |
|---|---|---|---|---|---|
| Number | Description | | | | |
| B1 | Anti- | 10 mM L- | 7% (w/v) | 0.02% | 10 mM |
| | CTLA4 | Histidine | Sucrose | (w/v) | L-Met |
| | antibody | buffer | | poly- | |
| | (50 mg/mL) | | | sorbate 80 | |
| B2 | Anti- | 10 mM | 7% (w/v) | 0.02% | 10 mM |
| | CTLA4 | Acetate | Sucrose | (w/v) | L-Met |
| | antibody | | | poly- | |
| | (50 mg/mL) | | | sorbate 80 | |

The vials were then incubated at three different storage conditions: 5° C. (ambient humidity), 25° C. (60% relative humidity), and 40° C. (75% relative humidity). Data was collected as follows:

TABLE 20
Schedule for Formulation B1

| | | | | | | Freeze | Agitation | | Light | | |
|---|---|---|---|---|---|---|---|---|---|---|---|
| | Initial | 2-week | 4-week | 6-week | 8-week | Thaw | (300 RPM) | | (ICH) | | |
| 5 C. | X | | X | | X | X | X | X | X | X | X |
| | | | | | | | | | (0.2) | (0.5) | (1) |
| 25 C. | | | X | | X | | | | | | |
| 40 C. | | X | X | X | X | | | | | | |

TABLE 21
Schedule for Formulation B2

| | | | | | | Freeze | Agitation | Light | | |
|---|---|---|---|---|---|---|---|---|---|---|
| | Initial | 2-week | 4-week | 6-week | 8-week | Thaw | (300 RPM) | (ICH) | | |
| 5 C. | X | | X | | X | | | X | X | X |
| | | | | | | | | (0.2) | (0.5) | (1) |
| 25 C. | | | X | | X | | | | | |
| 40 C. | | | X | X | X | | | | | |

Photostability studies were conducted using 1 mL liquid formulations of B1 and B2 in glass vials at room temperature under 0.2×ICH; 0.5×ICH; and 1×ICH.

Protein concentrations were measured by using UV absorbance at 280 nm.

Samples were equilibrated to room temperature and turbidity studies (A350) were conducted on the samples at spectrophotometric absorbance at 350 nm

Samples were assessed by size exclusion chromatography (SEC) for purity in which the percentage of monomer was determined, as well as the percentages of high molecular weight species (HMW) and late eluting peaks (LMW species). Ultra Performance-Size Exclusion Chromatography (UP-SEC) was performed by diluting the samples to 5.0 mg/mL in mobile phase (50 mM sodium phosphate, 450 mM arginine monohydrochloride, pH 7.0). The diluted samples were injected (6 μL) into a UPLC equipped with a Waters BEH200 column and a UV detector. Proteins in the sample were separated by size and detected by UV absorption at 280 nm.

Ion exchange chromatography was performed to evaluate the chemical stability and to monitor the change in the charge variant profile over time. An ion exchange HPLC method was performed using a Dionex ProPac WCX-10 column and a UV detector at 280 nm. Samples were diluted in purified water, and 80 μg were injected for analysis. The mobile phase used for the IEX analysis of the thermal stability samples was a gradient of the following mobile phases (mobile phase A: 20 mM MOPS, pH 7.2; mobile phase B: 50 mM sodium phosphate, 60 mM sodium chloride pH 8.0). The assay is performed using a mobile phase gradient from 20 mM MOPS, pH 7.2 to 50 mM sodium phosphate, 60 mM NaCl, pH 8.0. UV detection is performed at 280 nm. These methods are considered equivalent, and results are presented as relative percentages based on the total area of the chromatogram.

Peptide mapping was performed by Lys-C digestion. Samples were injected on Q Exactive at 30 μl/sample. The data analysis was done by PinPoint software.

The results of the studies are set forth in the tables below:

TABLE 22

| Storage Condition | 5° C. | | | | | |
|---|---|---|---|---|---|---|
| | Formulation B1 | | | Formulation B2 | | |
| Formulations | (L-Histidine Buffer) | | | (Acetate Buffer) | | |
| Time (weeks) | 0 | 4 | 8 | 0 | 4 | 8 |
| pH | 5.6 | 5.6 | 5.6 | 5.6 | 5.6 | 5.6 |
| Concentration (mg/mL) | 48.5 | 48.9 | 49.5 | 49.2 | 48.9 | 49.5 |
| (A₂₈₀ nm) | | | | | | |
| Turbidity (A₃₅₀ nm) | 0.071 | 0.069 | 0.068 | 0.068 | 0.071 | 0.067 |

HPSEC

| | | | | | | |
|---|---|---|---|---|---|---|
| High Molecular Weight | 1.17 | 1.20 | 1.24 | 1.15 | 1.19 | 1.17 |
| Species (%) | | | | | | |
| Low Molecular Weight | ND | ND | 0.01 | ND | ND | ND |
| Peaks (%) | | | | | | |
| Monomer (%) | 98.8 | 98.8 | 98.8 | 98.8 | 98.8 | 98.8 |

HP-IEX

| | | | | | | |
|---|---|---|---|---|---|---|
| Acidic variants (%) | 13.15 | 13.11 | 13.25 | 13.05 | 13.07 | 13.12 |
| Basic Variants (%) | 12.65 | 12.75 | 12.71 | 12.74 | 12.81 | 12.79 |
| Main (%) | 74.2 | 74.1 | 74.0 | 74.2 | 74.1 | 74.1 |

Peptide Mapping

| | | | | | | |
|---|---|---|---|---|---|---|
| % LC-M4 oxidation | 0.1 | NT | NT | 0.1 | NT | NT |
| % HC-M34 oxidation | 0.2 | NT | NT | 0.2 | NT | NT |
| % HC-M250 oxidation | 1.2 | NT | NT | 1.3 | NT | NT |
| % HC-M426 oxidation | 0.6 | NT | NT | 0.6 | NT | NT |

TABLE 23

| Storage Condition | 25° C. | | | | | |
|---|---|---|---|---|---|---|
| | Formulation B1 | | | Formulation B2 | | |
| Formulations | (L-Histidine Buffer) | | | (Acetate Buffer) | | |
| Time (weeks) | 0 | 4 | 8 | 0 | 4 | 8 |
| pH | 5.6 | 5.6 | 5.6 | 5.6 | 5.6 | 5.6 |
| Concentration (mg/mL) | 48.5 | 49.1 | 49.2 | 49.2 | 49.0 | 49.5 |
| (A₂₈₀ nm) | | | | | | |
| Turbidity (A₃₅₀ nm) | 0.071 | 0.071 | 0.074 | 0.068 | 0.069 | 0.073 |

HPSEC

| | | | | | | |
|---|---|---|---|---|---|---|
| High Molecular Weight | 1.17 | 1.28 | 1.34 | 1.15 | 1.29 | 1.33 |
| Species (%) | | | | | | |
| Low Molecular Weight | ND | 0.06 | 0.12 | ND | 0.06 | 0.11 |
| Peaks (%) | | | | | | |
| Monomer (%) | 98.8 | 98.7 | 98.6 | 98.8 | 98.7 | 98.6 |

HP-IEX

| | | | | | | |
|---|---|---|---|---|---|---|
| Acidic variants (%) | 13.15 | 15.10 | 17.50 | 13.05 | 15.73 | 18.77 |
| Basic Variants (%) | 12.65 | 13.39 | 13.87 | 12.74 | 13.41 | 13.96 |
| Main (%) | 74.2 | 71.5 | 68.6 | 74.2 | 70.9 | 67.3 |

Peptide Mapping

| | | | | | | |
|---|---|---|---|---|---|---|
| % LC-M4 oxidation | 0.1 | NT | NT | 0.1 | NT | NT |
| % HC-M34 oxidation | 0.2 | NT | NT | 0.2 | NT | NT |
| % HC-M250 oxidation | 1.2 | NT | NT | 1.3 | NT | NT |
| % HC-M426 oxidation | 0.6 | NT | NT | 0.6 | NT | NT |

TABLE 24

| Storage Condition | 40° C. | | | | | |
|---|---|---|---|---|---|---|
| | Formulation B1 | | | Formulation B2 | | |
| Formulations | (L-Histidine Buffer) | | | (Acetate Buffer) | | |
| Time (weeks) | 0 | 4 | 8 | 0 | 4 | 8 |
| pH | 5.6 | 5.6 | 5.6 | 5.6 | 5.6 | 5.6 |
| Concentration (mg/mL) | 48.5 | 48.7 | 49.3 | 49.2 | 48.8 | 49.4 |
| (A₂₈₀ nm) | | | | | | |
| Turbidity (A₃₅₀ nm) | 0.071 | 0.081 | 0.101 | 0.068 | 0.085 | 0.094 |

HPSEC

| | | | | | | |
|---|---|---|---|---|---|---|
| High Molecular Weight | 1.17 | 1.34 | 1.47 | 1.15 | 1.42 | 1.60 |
| Species (%) | | | | | | |
| Low Molecular Weight | ND | 0.65 | 1.47 | ND | 0.58 | 1.38 |
| Peaks (%) | | | | | | |
| Monomer (%) | 98.8 | 98.0 | 97.0 | 98.8 | 98.0 | 97.0 |

HP-IEX

| | | | | | | |
|---|---|---|---|---|---|---|
| Acidic variants (%) | 13.15 | 29.60 | 44.85 | 13.05 | 32.83 | 49.48 |
| Basic Variants (%) | 12.65 | 15.77 | 15.55 | 12.74 | 15.03 | 14.74 |
| Main (%) | 74.2 | 54.6 | 39.6 | 74.2 | 52.1 | 35.8 |

Peptide Mapping

| | | | | | | |
|---|---|---|---|---|---|---|
| % LC-M4 oxidation | 0.1 | NT | 0.1 | 0.1 | NT | 0.1 |
| % HC-M34 oxidation | 0.2 | NT | 0.2 | 0.2 | NT | 0.2 |
| % HC-M250 oxidation | 1.2 | NT | 1.4 | 1.3 | NT | 1.7 |
| % HC-M426 oxidation | 0.6 | NT | 0.7 | 0.6 | NT | 0.7 |

TABLE 25

| Formulations | Formulation B1 (L-Histidine Buffer) | | | | | | |
|---|---|---|---|---|---|---|---|
| | | | Agitation | | Photostability (ICH) | | |
| Stress | T0 | Freeze-Thaw | 1 d | 3 d | 0.2X | 0.5X | 1X |
| pH | 5.6 | 5.6 | 5.6 | 5.6 | 5.6 | 5.6 | 5.5 |
| Concentration (mg/mL) | 48.5 | 49.6 | 49.0 | 49.4 | 49.2 | 49.2 | 49.6 |
| (A₂₈₀ nm) | | | | | | | |
| Turbidity (A₃₅₀ nm) | 0.071 | 0.069 | 0.067 | 0.07 | 0.081 | 0.137 | 0.193 |

HPSEC

| | | | | | | | |
|---|---|---|---|---|---|---|---|
| High Molecular Weight | 1.17 | 1.19 | 1.20 | 1.20 | 1.62 | 2.00 | 3.01 |
| Species (%) | | | | | | | |
| Low Molecular Weight | ND | ND | ND | 0.01 | 0.02 | 0.07 | 0.16 |
| Peaks (%) | | | | | | | |
| Monomer (%) | 98.8 | 98.8 | 98.8 | 98.8 | 98.4 | 97.9 | 96.8 |

HP-IEX

| | | | | | | | |
|---|---|---|---|---|---|---|---|
| Acidic variants (%) | 13.15 | 13.29 | 13.19 | 13.30 | 15.53 | 18.36 | 23.66 |
| Basic Variants (%) | 12.65 | 12.91 | 12.75 | 12.89 | 19.70 | 25.85 | 34.22 |
| Main (%) | 74.2 | 73.8 | 74.1 | 73.8 | 64.8 | 55.8 | 42.1 |

Peptide Mapping

| | | | | | | | |
|---|---|---|---|---|---|---|---|
| % LC-M4 oxidation | 0.1 | NT | NT | NT | 0.2 | 0.3 | 0.6 |
| % HC-M34 oxidation | 0.2 | NT | NT | NT | 0.3 | 0.3 | 0.4 |
| % HC-M250 oxidation | 1.2 | NT | NT | NT | 6.2 | 11.5 | 22.7 |
| % HC-M426 oxidation | 0.6 | NT | NT | NT | 4.6 | 8.8 | 18.8 |

TABLE 26

| Formulations | Formulation B2 (Acetate Buffer) | | | | | | |
|---|---|---|---|---|---|---|---|
| | | | Agitation | | Photostability (ICH) | | |
| Stress | T0 | Freeze-Thaw | 1d | 3d | 0.2X | 0.5X | 1X |
| pH | 5.6 | 5.6 | 5.6 | 5.6 | 5.6 | 5.6 | 5.6 |
| Concentration (mg/mL) | 49.18 | 49.4 | 49.1 | 48.8 | 49.0 | 49.1 | 49.6 |
| (A₂₈₀ nm) | | | | | | | |
| Turbidity (A₃₅₀ nm) | 0.068 | 0.066 | 0.066 | 0.069 | 0.075 | 0.095 | 0.110 |

HPSEC

| | | | | | | | |
|---|---|---|---|---|---|---|---|
| High Molecular Weight | 1.15 | 1.18 | 1.16 | 1.16 | 2.00 | 3.12 | 4.69 |
| Species (%) | | | | | | | |
| Low Molecular Weight | ND | ND | ND | ND | 0.02 | 0.09 | 0.17 |
| Peaks (%) | | | | | | | |
| Monomer (%) | 98.8 | 98.8 | 98.8 | 98.8 | 98.0 | 96.8 | 95.1 |

HP-IEX

| | | | | | | | |
|---|---|---|---|---|---|---|---|
| Acidic variants (%) | 13.05 | 13.17 | 13.16 | 13.15 | 14.30 | 16.39 | 18.33 |
| Basic Variants (%) | 12.74 | 13.12 | 12.89 | 12.70 | 22.07 | 30.87 | 39.93 |
| Main (%) | 74.2 | 73.7 | 74.0 | 74.1 | 63.6 | 52.7 | 41.7 |

Peptide Mapping

| | | | | | | | |
|---|---|---|---|---|---|---|---|
| % LC-M4 oxidation | 0.1 | NT | NT | NT | 0.3 | 0.5 | 0.8 |
| % HC-M34 oxidation | 0.2 | NT | NT | NT | 0.3 | 0.4 | 0.5 |
| % HC-M250 oxidation | 1.3 | NT | NT | NT | 7.9 | 16.0 | 29.1 |
| % HC-M426 oxidation | 0.6 | NT | NT | NT | 6.2 | 12.5 | 26.1 |

There were no measurable changes in protein concentration between the formulations for the conditions and duration of the study, as measured by UV absorbance at 280 nm.

There were no measurable changes in the pH between the formulations for the conditions tested and the duration of the study.

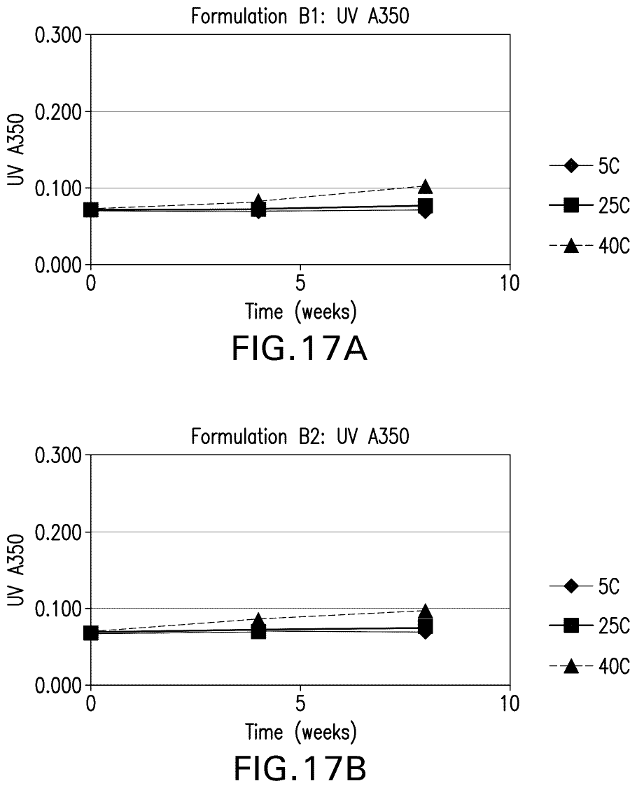

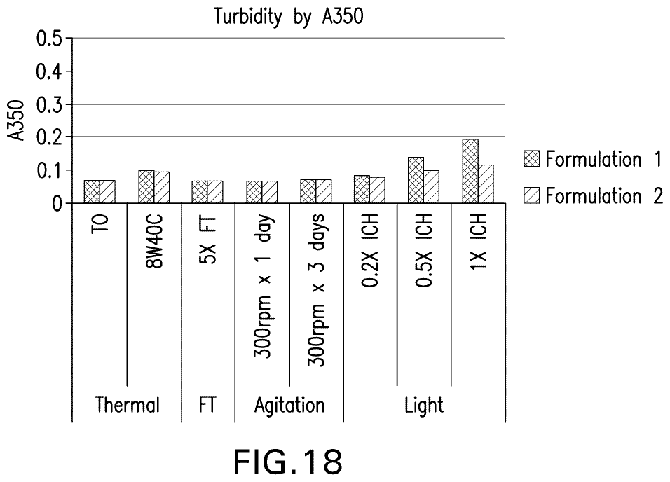

Turbidity (A350) data is shown in FIG. 17A FIG. 17B and FIG. 18. Upon comparing the data, it was found that at 40° C., both the formulations showed a trend of increase in turbidity for up to 8-week time point. At 25° C. and 5° C., both formulations showed no substantial changes in turbidity for up to 8-week time point. As shown in FIG. 14 it was also observed that Formulation B1 showed a slightly more, but consistent, increase in turbidity following light-stress conditions (0.5×ICH and 1×ICH) as compared to Formulation B2. Treatment of samples to either freeze-thaw (up to 5×) or agitation stresses (300 rpm, for up to 3 days) did not change sample turbidity compared to control (TO) samples.

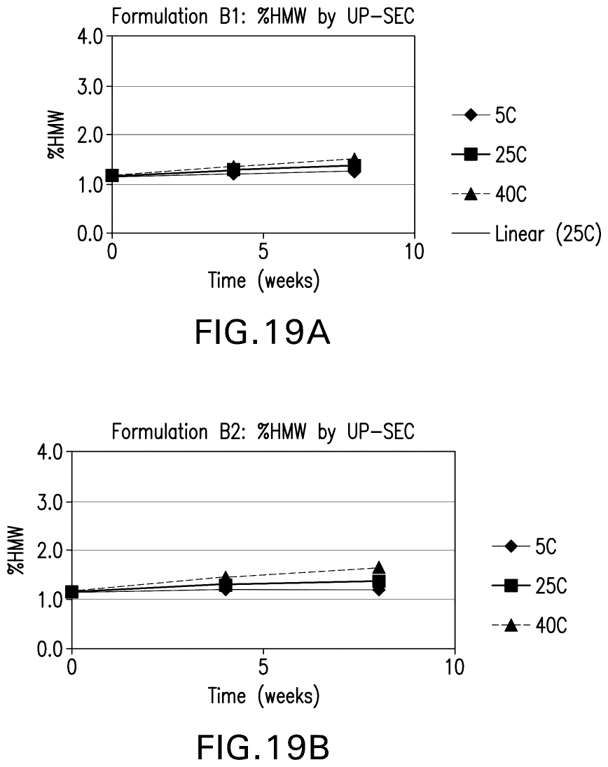

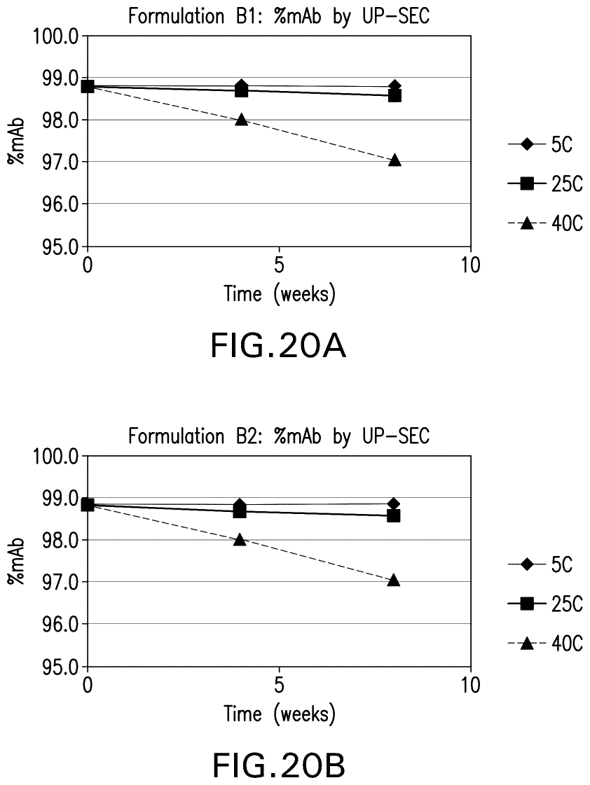

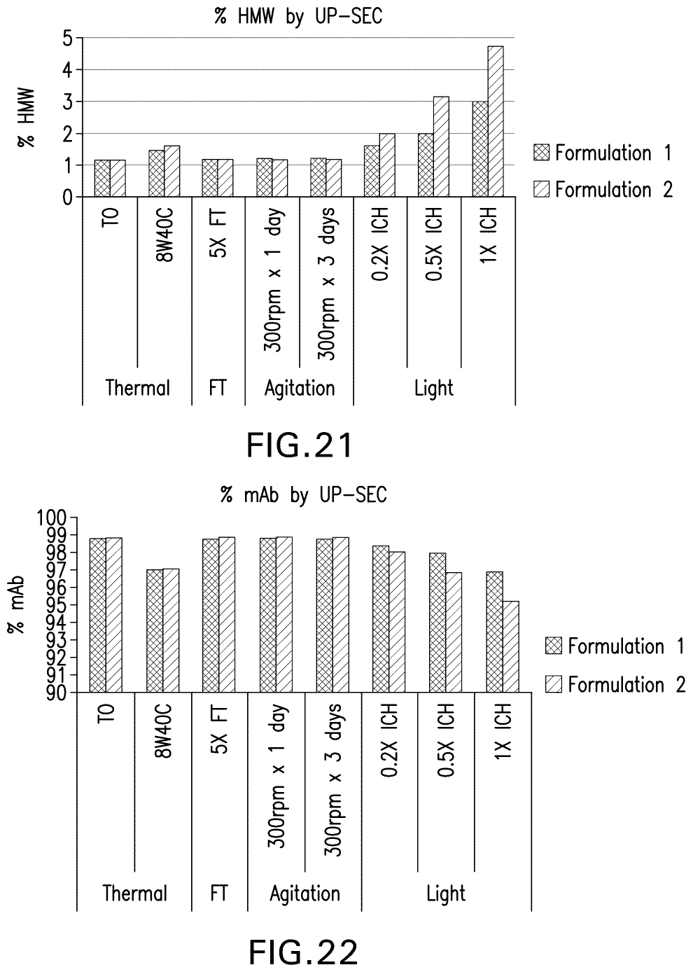

As shown in FIGS. 19A, 19B, 20A, and 20B, UP-SEC analysis of the samples to determine the percentage of HMW and percentage of monomer indicated that at 40° C. both the formulations showed a trend of increase in % HMW peak and % LMW peak (and a consequent decrease in % monomer peak) for up to 8-week time point. At 25° C., both the formulations showed similar trends, but smaller changes, as compared to 40° C. At 5° C., no substantial changes were observed. As shown in FIGS. 21 and 22, Formulation B2 shows a slightly more (but consistent) increase in % HMW (and corresponding slightly more, but consistent, decrease in % monomer) for all light-stress conditions studied as compared to Formulation B1. Treatment of samples to either freeze-thaw (up to 5×) or agitation stresses (300 rpm, for up to 3 days) did not change % HMW or % monomer compared to control (TO) samples (FIGS. 21 and 22)

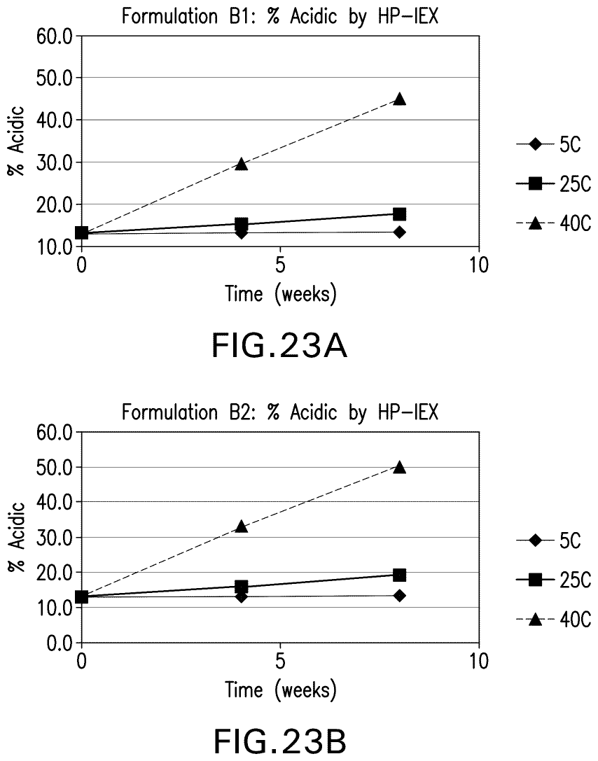

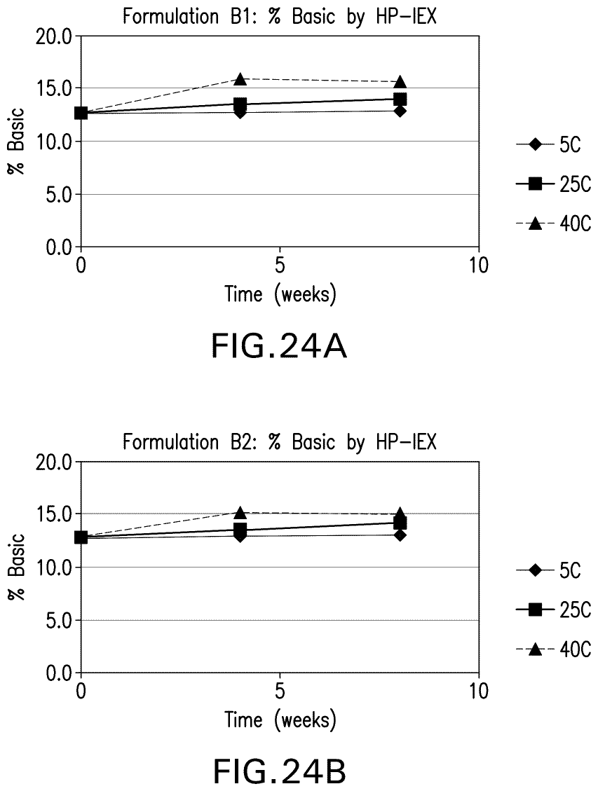

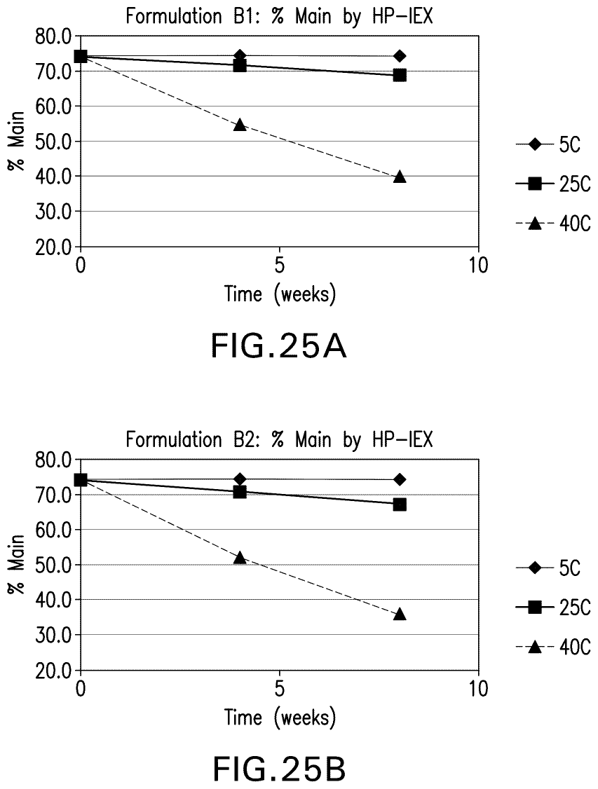

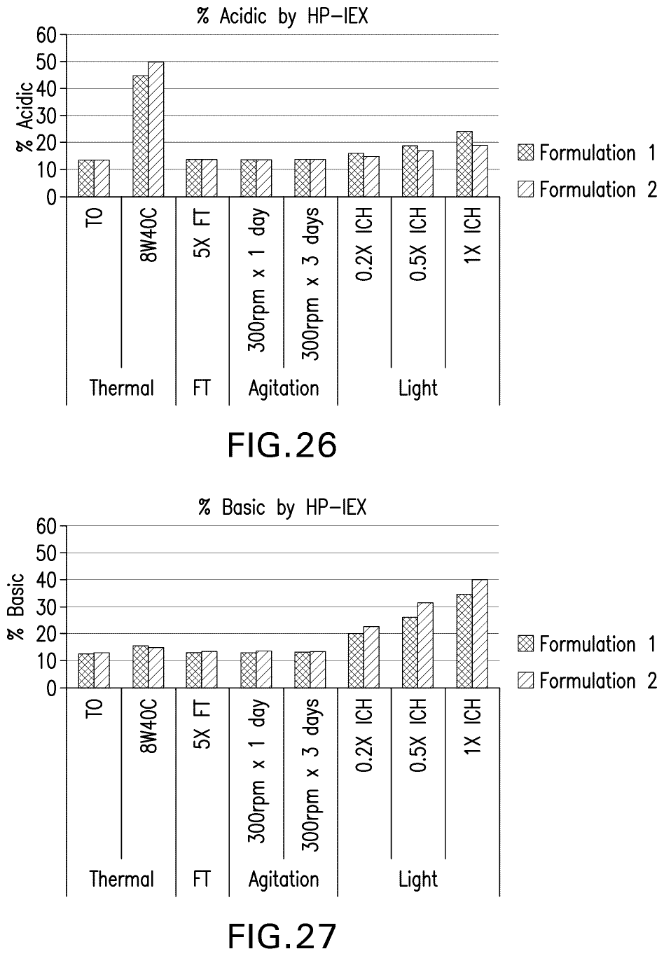

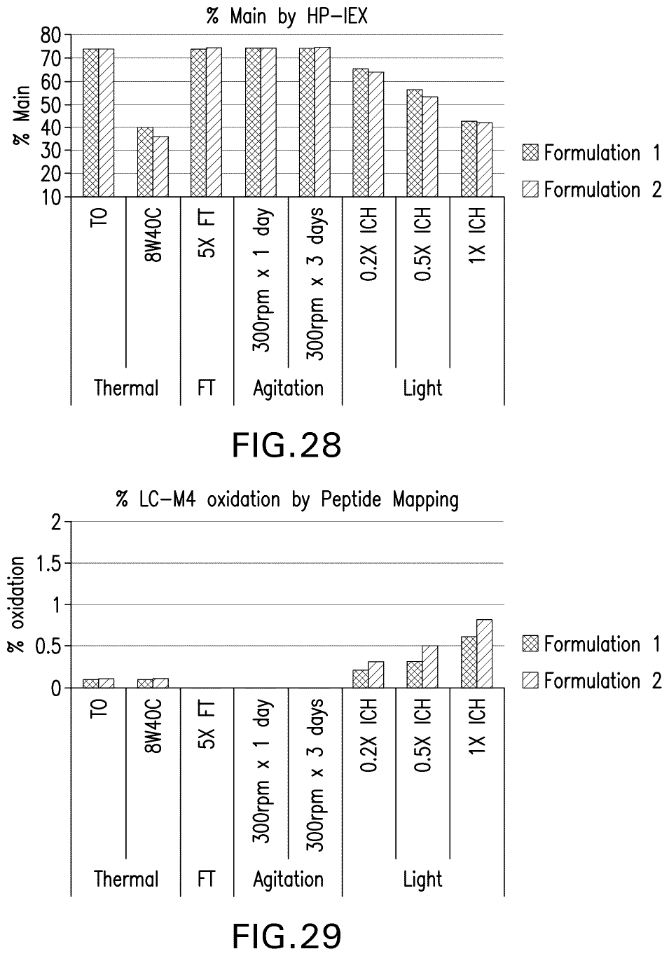

As shown in FIGS. 23A, 23B, 24A, 24B, 25A and 25B, the HP-IEX data indicates that at 40° C., both the formulations showed a trend of increase in % acidic peak and % basic peak up to 8-week time point, along with a corresponding trend of decrease in the % main peak. However, Formulation B2 showed a slightly more, but consistent, decrease of % main peak as compared to Formulation B1. At 25° C., both the formulations showed similar trends, but smaller changes, as compared to 40° C. As shown in FIGS. 26, 27, and 28, following light-stress conditions (0.5×ICH and 1×ICH), Formulation B1 shows a slightly more, but consistent, increase in % acidic peaks, while Formulation B2 shows a slightly more (but consistent) increase in % basic peaks. However, corresponding decrease in % main peak was comparable for the two formulations. Treatment of samples to either freeze-thaw (up to 5×) or agitation stresses (300 rpm, for up to 3 days) did not change % acidic peak, % basic peak, or % main peak compared to control (TO) samples (FIGS. 26-28).

Peptide mapping studies were performed to determine the changes in oxidation level of these residues upon 8 week exposure at 40° C. or 0.2×/0.5×/1×ICH light stress treatment.

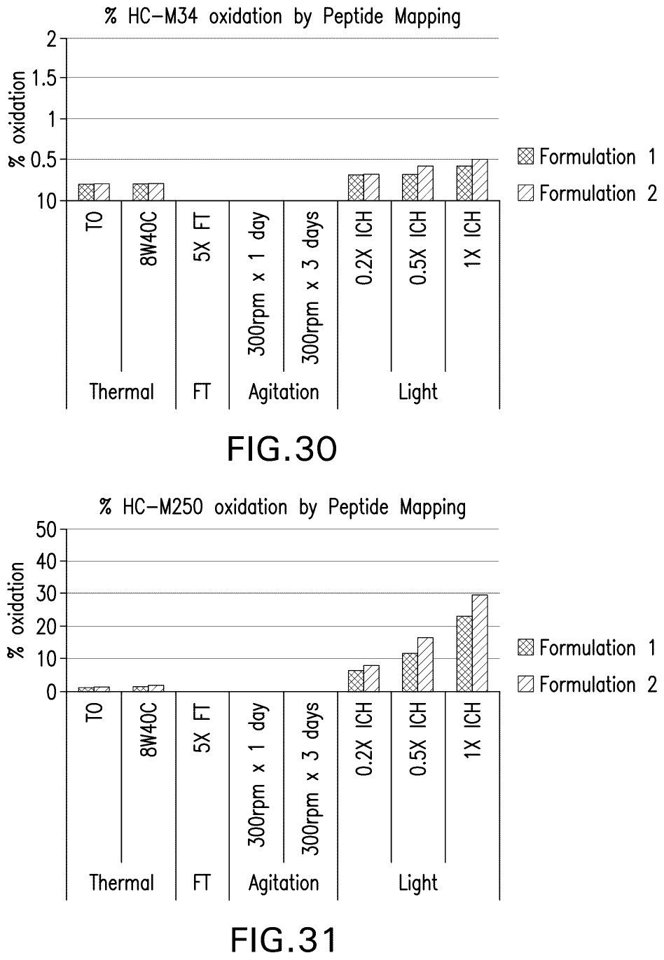

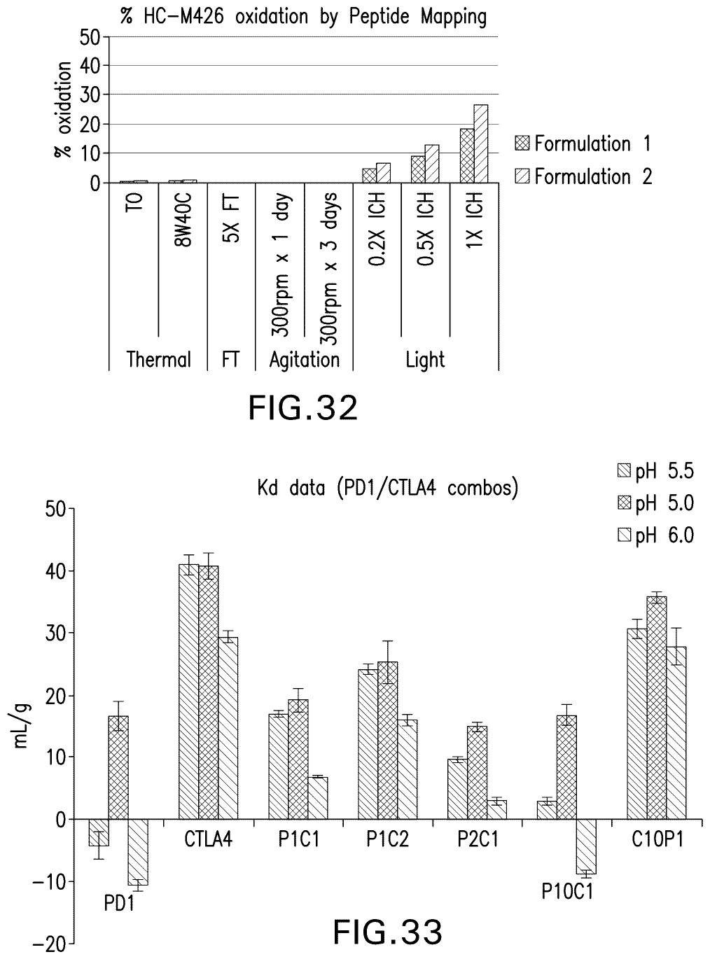

The results of the peptide mapping studies showing oxidation percent oxidation in residues LC-M4, HC-M34, HC-M250 and HC-M426 are represented in FIG. 29, FIG. 30, FIG. 31 and FIG. 32 respectively.

As shown in FIGS. 29-32, under light stress conditions there increased oxidation of certain methionine residues. Notably, residues HC-M250 and HC-M426 showed significant increase in oxidation levels upon 0.5× and 1×ICH light stress treatment in both formulations. However, Formulation B1 resulted in smaller increase in oxidation levels of M250 and M426 as compared to Formulation B2.

Based on a comparison of the analytical data from the above studies, formulation B1 (L-histidine buffer) as compared to formulation B2 (acetate buffer) exhibited (i) lower increase in aggregate levels (% HMW) for all light-stress conditions, (ii) slightly less, but consistent, decrease in the % main peak at 40 C, and (iii) lower increase in oxidation levels of residues HC M250 and HC M426 following light stress. Hence, based on the protein-protein interaction data and the stability data, it is shown that the anti-CTLA4 antibody is stable in both the L-histidine buffer and the acetate buffer.

### Example 3

### Co-Formulation of an Anti-CTLA4 Antibody and an Anti-PD-1 Antibody.

Co-formulation of two antibodies into a single formulation is more convenient for patients and increases patient compliance. Based on the protein-protein interactions (shown below), all the co-formulations (shown below) were found to be stable across pH 5.0-6.0. Hence, three co-formulations (P1C1, P1C2 and P2C1) at pH 5.5 were chosen and placed on additional thermal stability at 5° C., 25° C. and 40° C. along with the two controls (anti-PD1 and anti-CTLA4).

This study evaluates the stability of an anti-CLTA4 antibody having the followings CDRs: HCDR1 of SEQ ID NO:35, HCDR2 of SEQ ID NO: 36, HCDR3 of SEQ ID NO: 37, LCDR1 of SEQ ID NO: 38, LCDR2 of SEQ ID NO: 39, and LCDR3 of SEQ ID NO: 40 on an IgG1 backbone co-formulated with pembrolizumab at various concentrations as follows:

TABLE 27

| | Pembrolizumab/ | | Anti- | Total |
|---|---|---|---|---|
| Co- | Anti-CTLA4 | | CTLA4 | Con- |
| formulations | ratio (w/w) | Pembro | antibody | centration |
| P2C1 | 2:1 | 25 mg/mL | 12.5 mg/mL | 37.5 mg/mL |
| P1C1 | 1:1 | 25 mg/mL | 25 mg/mL | 50 mg/mL |
| P1C2 | 1:2 | 25 mg/mL | 50 mg/mL | 75 mg/mL |
| P1C10 | 1:10 | 22.72 mg/mL | 2.3 mg/mL | 25 mg/mL |
| P10C1 | 1:10 | 2.27 mg/mL | 22.7 mg/mL | 25 mg/mL |
| P1 (Control) | 1:0 | 25 mg/mL | None | 25 mg/mL |
| C1 (Control) | 0:1 | None | 50 mg/mL | 50 mg/mL |
| 25/200 | 8:1 | 23.5 mg/mL | 2.9 mg/mL | 26.4 mg/mL |
| 75/200 | 8:3 | 21.1 mg/mL | 7.9 mg/mL | 29.0 mg/mL |

The formulations were prepared as liquid formulations as follows:

TABLE 28

| | | | Cryoprotectant/ | | |
|---|---|---|---|---|---|
| | | | Tonicity | | Anti- |
| Formulation | Buffer | pH | modifier | Surfactant | oxidant |
| P2C1 | L-Histidine | 5, 5.5, 6 | Sucrose (7%) | PS-80 | 10 mM |
| | (10 mM) | | | (0.02%) | L-Met |
| P1C1 | L-Histidine | 5, 5.5, 6 | Sucrose (7%) | PS-80 | 10 mM |
| | (10 mM) | | | (0.02%) | L-Met |
| P1C2 | L-Histidine | 5, 5.5, 6 | Sucrose (7%) | PS-80 | 10 mM |
| | (10 mM) | | | (0.02%) | L-Met |
| P1C10 | L-Histidine | 5, 5.5, 6 | Sucrose (7%) | PS-80 | 10 mM |
| | (10 mM) | | | (0.02%) | L-Met |
| P10C1 | L-Histidine | 5, 5.5, 6 | Sucrose (7%) | PS-80 | 10 mM |
| | (10 mM) | | | (0.02%) | L-Met |
| P1 (Control) | L-Histidine | 5, 5.5, 6 | Sucrose (7%) | PS-80 | 10 mM |
| | (10 mM) | | | (0.02%) | L-Met |
| C1 (Control) | L-Histidine | 5, 5.5, 6 | Sucrose (7%) | PS-80 | 10 mM |
| | (10 mM) | | | (0.02%) | L-Met |

Each formulation was filled at 1 mL into 2R vials. Stability was measured by visual inspection, turbidity by PD350, protein concentration, Microwflow Imaging (MFI) (evaluation of particulates), mixed mode size exclusion chromatography (SEC) (evaluation of aggregation), cIEF (evaluation of charge variants), IEX (evaluation of charge variants), and UP-SEC (evaluation of aggregation). Because anti-CTLA4 and anti-PD1 are co-eluted in UP-SEC, for the co-formulation, anti-PD1 and anti-CTLA4 were separated by mixed mode SEC to evaluate stability of coformulation. Co-formulation at pH 5.5 were used in the thermal stability studies. The thermal stability protocol is as follows:

TABLE 29

| | T0 | 1 month | 3 Month | 6 Month | 9 month | 12 Month | Extra |
|---|---|---|---|---|---|---|---|
| 5° C. | 3 combo + | 3 combo + | 3 combo + | 3 combo + | 3 combo | 3 combo + | 9 combo + |
| (ambient | 2 mono | 2 mono | 2 mono | 2 mono | | 2 mono | 9 mono |
| humitidy) | | | | | | | |
| 25° C. | | 3 combo + | 3 combo + | 3 combo + | 3 combo | 3 combo + | N/A |
| (60% | | 2 mono | 2 mono | 2 mono | | 2 mono | |
| relative | | | | | | | |
| humidity) | | | | | | | |
| 40° C. | | 3 combo + | 3 combo + | 3 combo + | N/A | N/A | N/A |
| (75% | | 2 mono | 2 mono | 2 mono | | | |
| relative | | | | | | | |
| humidity) | | | | | | | |

Protein-protein interactions, which are indicative of colloidal and thermal stability, of the different co-formulations were measures (3 co-formulations and 2 controls). A repulsive protein-protein interaction, as indicated by a positive diffusion interaction parameter (K_{D}) value of K_{D}>0 indicates a stable formulation with low propensity for aggregation. The Kd for all formulations at here different pH values (pH 5.0, 5.5, and 6.0) were measured at least three times to obtain a standard deviation. As shown in FIG. 33, combos P1C1, P2C2, and P1C2 are stable at each of the three pH values since all the co-formulations have positive K_{D} values. Pembrolizumab (PD1) and pembrolizumab rich combinations (P2C1) are more stable at pH of 5.0 as compared to CTLA4 rich combinations (P1C2). The CTLA4-rich combinations (P1C2) are equally stable at pH 5.0 and pH 5.5. That is, co-formulations with a greater fraction of pembrolizumab are more stable at a pH of 5.0 as compared to co-formulations with a greater fraction of CTLA4, which are equally stable at pH 5.0 and 5.5.

### 12 Month Stability Results

At twelve months, not much change in total protein concentration (determined by UV 280) was observed at any condition (data not shown).

Turbidity changes: Turbidity changes were observed for up to 12 months in the following order: 5° C.<25° C.<40° C. for each formulation. The rate of change of turbidity at 40° C. for up to 6 months data appeared directly proportional to the total protein concentration in each formulation. (data not shown).

The number of particulates (measured by MFI) in all formulation at all conditions appears fairly low (data not shown). Microflow (MFI) imaging was used to characterize the co-formulated samples. One milliliter of sample is drawn into a pipet tip, and gently pumped (0.17 mL/min) through a flow cell (150 micron depth of field) of a microscopic system to enable particle count and image capture of particles by a digital camera. As the samples passes through the flow cell, bright-field images are captured in real time in continuous succession. The output at the end of the analysis, is particle count and particle concentration data. MFI images can also be processed using system software for different morphological parameters, such as size, intensity, transparency and shape.

For each of the anti-PD-1 and anti-CTLA4 antibodies, an increase in % acidic charge variants, indicative of deamidation, for each antibody was observed at higher temperatures (25° and 40° C.). An ion exchange HPLC method was performed using a Dionex ProPac WCX-10 column and a UV detector at 280 nm. Samples were diluted in purified water, and 80 μg were injected for analysis. The mobile phase used for the IEX analysis of the thermal stability samples was a gradient of the following mobile phases (mobile phase A: 24 mM MES pH 6, 4% acetonitrile; mobile phase B: 20 mM NaPO4, 95 mM NaCl pH 8, 4% acetonitrile). A summary of the normalized cIEF data (initial, 3 and 6 months) is listed below:

TABLE 30

| | | aPD1 | aPD1 | aPD1 | aCTLA | aCTLA | aCTLA |
|---|---|---|---|---|---|---|---|
| Sample | | acidic | Main | basic | acidic | main | basic |
| P2C1 | initial | 20.83 | 66.99 | 12.18 | 19.84 | 75.13 | 5.03 |
| P1C1 | initial | 20.24 | 66.38 | 13.38 | 19.59 | 75.26 | 5.15 |
| P1C2 | initial | 18.10 | 67.84 | 14.06 | 18.86 | 75.77 | 5.37 |
| P1 | initial | 23.70 | 63.24 | 13.07 | | | |
| C1H | initial | | | | 20.12 | 73.70 | 6.18 |
| P2C1 | 3M5C | 21.18 | 65.94 | 12.88 | 16.07 | 78.16 | 5.77 |
| P1C1 | 3M5C | 20.22 | 66.79 | 12.98 | 24.71 | 68.71 | 6.59 |
| P1C2 | 3M5C | 18.51 | 68.83 | 12.66 | 17.06 | 77.63 | 5.30 |
| P1 | 3M5C | 24.12 | 63.15 | 12.73 | | | |
| C1H | 3M5C | | | | 28.13 | 65.33 | 6.54 |
| P2C1 | 3M25C | 25.44 | 62.77 | 11.79 | 24.03 | 69.51 | 6.46 |
| P1C1 | 3M25C | 24.15 | 64.99 | 10.86 | 24.35 | 69.31 | 6.34 |
| P1C2 | 3M25C | 24.63 | 65.60 | 9.77 | 29.18 | 64.52 | 6.30 |
| P1 | 3M25C | 28.97 | 60.41 | 10.62 | | | |
| C1H | 3M25C | | | | 27.23 | 65.63 | 7.14 |
| P2C1 | 3M40C | 59.56 | 36.14 | 4.30 | 23.94 | 39.68 | 36.38 |
| P1C1 | 3M40C | 59.24 | 36.22 | 4.54 | 62.92 | 31.53 | 5.55 |
| P1C2 | 3M40C | 56.68 | 39.25 | 4.07 | 63.29 | 31.17 | 5.55 |
| P1 | 3M40C | 43.81 | 50.20 | 5.99 | | | |
| C1H | 3M40C | | | | 60.31 | 34.14 | 5.55 |
| P2C1 | 6M5C | 21.57 | 35.69 | 7.08 | 7.29 | 25.82 | 2.55 |
| P1C1 | 6M5C | 15.98 | 38.73 | 9.64 | 10.69 | 22.35 | 2.6 |
| P1C2 | 6M5C | 16.62 | 26.47 | 7.65 | 14.02 | 31.67 | 3.59 |
| P1 | 6M5C | 36.84 | 49.21 | 13.94 | | | |
| C1H | 6M5C | | | | 27.56 | 67.01 | 5.45 |
| P2C1 | 6M25C | 20.09 | 29.69 | 6.34 | 13.71 | 27.5 | 2.67 |
| P1C1 | 6M25C | 28.35 | 29.98 | 8.46 | 13.17 | 18.09 | 1.95 |
| P1C2 | 6M25C | 21.9 | 21.66 | 5.41 | 21 | 26.15 | 3.87 |
| P1 | 6M25C | 45.43 | 46.07 | 8.52 | | | |
| C1H | 6M25C | | | | 35.46 | 57.47 | 7.05 |
| P2C1 | 6M40C | 56.37 | 7.93 | 2.3 | 27.97 | 5.44 | 0 |
| P1C1 | 6M40C | 38.81 | 16.47 | 9.93 | 9.039 | 10.94 | 9.37 |
| P1C2 | 6M40C | 28.56 | 4.33 | 2.74 | 55.77 | 6.7 | 1.93 |
| P1 | 6M40C | 83.33 | 12.15 | 4.53 | | | |
| C1H | 6M40C | | | | 89.15 | 8 | 2.86 |

Aggregation was measured using UPSEC and mixed mode SEC. Although the anti-CTLA4 and anti-PD1 antibody co-elute in UP-SEC, they are separated and visualized by the mixed mode SEC. HMW aggregates and LMW aggregates increased with temperature. UP-SEC results indicated that very low percentage of aggregates (% HMW) species were detected up to 12 months at 5° C. and 25° C. and there are no major changes as compared to the initial time point.

TABLE 31

| | HMW % | LMW % | Main % |
|---|---|---|---|
| Sample | Initial Value (T0) | Initial Value (T0) | Initial Value (T0) |
| C1H | 0.93 | 0 | 99.07 |
| P1 | 0.53 | 0 | 99.47 |
| P1C2 | 1.12 | 0 | 98.88 |
| P1C1 | 0.89 | 0 | 99.11 |
| P2C1 | 0.73 | 0 | 99.27 |

| | 1 M | 1 M | 1 M | 1 M | 1 M | 1 M | 1 M | 1 M | 1 M |
|---|---|---|---|---|---|---|---|---|---|
| Sample | 5° C. | 25° C. | 40° C. | 5° C. | 25° C. | 40° C. | 5° C. | 25° C. | 40° C. |
| C1H | 0.93 | 1.21 | 0.80 | 0.00 | 0.08 | 0.47 | 99.1 | 99.1 | 98.7 |
| P1 | 0.48 | 0.56 | 0.63 | 0.00 | 0.00 | 0.00 | 99.5 | 99.5 | 99.4 |
| P1C2 | 0.95 | 0.99 | 1.68 | 0.00 | 0.00 | 0.32 | 98.9 | 99.1 | 99.0 |
| P1C1 | 0.71 | 0.79 | 1.33 | 0.00 | 0.00 | 0.24 | 99.1 | 99.3 | 99.2 |
| P2C1 | 0.65 | 0.61 | 1.20 | 0.00 | 0.00 | 0.16 | 99.3 | 99.4 | 99.4 |
| | 3 M | 3 M | 3 M | 3 M | 3 M | 3 M | 3 M | 3 M | 3 M |
| Sample | 5° C. | 25° C. | 40° C. | 5° C. | 25° C. | 40° C. | 5° C. | 25° C. | 40° C. |
| C1H | 0.82 | 0.84 | 1.32 | 0 | 0.15 | 1.21 | 99.18 | 99.01 | 97.46 |
| P1 | 0.56 | 0.49 | 0.9 | 0 | 0 | 0.03 | 99.4 | 99.51 | 99.07 |
| P1C2 | 0.84 | 1.2 | 2.04 | 0 | 0.16 | 0.95 | 99.16 | 98.64 | 97.07 |
| P1C1 | 0.59 | 0.83 | 1.53 | 0 | 0 | 0.8 | 99.41 | 99.04 | 97.67 |
| P2C1 | 0.52 | 0.52 | 0.98 | 0 | 0.02 | 0.37 | 99.48 | 99.45 | 98.65 |
| | 6 M | 6 M | 6 M | 6 M | 6 M | 6 M | 6 M | 6 M | 6 M |
| Sample | 5° C. | 25° C. | 40° C. | 5° C. | 25° C. | 40° C. | 5° C. | 25° C. | 40° C. |
| C1H | 0.68 | 0.71 | 0.77 | 0 | 0.3 | 1.5 | 99.32 | 99 | 97.74 |
| P1 | 0.66 | 0.48 | 1.13 | 0 | 0 | 0 | 99.34 | 99.52 | 98.87 |
| P1C2 | 0.66 | 0.9 | 1.87 | 0 | 0.15 | 1.29 | 99.34 | 98.95 | 96.83 |
| P1C1 | 0.62 | 0.72 | 1.58 | 0 | 0 | 1 | 99.38 | 99.28 | 97.41 |
| P2C1 | 0.58 | 0.62 | 1.25 | 0 | 0 | 0.67 | 99.42 | 99.38 | 98.08 |
| | 9 M | 9 M | 9 M | 9 M | 9 M | 9 M | 9 M | 9 M | 9 M |
| Sample | 5° C. | 25° C. | 40° C. | 5° C. | 25° C. | 40° C. | 5° C. | 25° C. | 40° C. |
| C1H | 1.31 | 1.35 | ND | 0.08 | 0.77 | ND | 98.62 | 97.88 | ND |
| P1 | 0.7 | 0.86 | ND | 0 | 0.07 | ND | 99.3 | 99.07 | ND |
| P1C2 | 1.28 | 1.63 | ND | 0.09 | 0.54 | ND | 98.63 | 97.83 | ND |
| P1C1 | 1.04 | 1.3 | ND | 0.08 | 0.43 | ND | 98.88 | 98.28 | ND |
| P2C1 | 0.91 | 1.14 | ND | 0.08 | 0.34 | ND | 99.01 | 98.52 | ND |
| | 12 M | 12 M | 12 M | 12 M | 12 M | 12 M | 12 M | 12 M | 12 M |
| Sample | 5° C. | 25° C. | 40° C. | 5° C. | 25° C. | 40° C. | 5° C. | 25° C. | 40° C. |
| C1H | 1.34 | 1.38 | ND | 0.09 | 0.94 | ND | 98.57 | 97.69 | ND |
| P1 | 0.68 | 0.98 | ND | 0 | 0.09 | ND | 99.32 | 98.92 | ND |
| P1C2 | 1.31 | 1.75 | ND | 0.07 | 0.72 | ND | 98.62 | 97.54 | ND |
| P1C1 | 1.05 | 1.39 | ND | 0.06 | 0.54 | ND | 98.9 | 98.06 | ND |
| P2C1 | 0.9 | 1.23 | ND | 0.06 | 0.44 | ND | 99.04 | 98.34 | ND |

Mixed-Mode SEC was used to analyze the two antibodies in the co-formulation for aggregates and oxidation species. The results are set forth in the table below. Pembrolizumab showed an increase of oxidized species 1 and 2 at high temperatures, reflecting the oxidation of M105 on one and two arms, respectively. M105 is in CDR3 of pembrolizumab. An exposed methionine residue or a methionine residue in the CDR of an antibody has the potential of impacting the biological activity of the antibody through oxidation. Methionine reduces the oxidation of Met105 within the pembrolizumab heavy chain CDR. Minor changes in oxidation species as compared to the initial indicates that the co-formulation is stable up to 12 months at 5° C.

| | | | | Pembrolizumab | | | | |
|---|---|---|---|---|---|---|---|---|
| | Anti-CTLA4 Antibody | | | | Met105 | Met105 | | |
| Sample | % HMW | Monomer | % LMW | % HMW | (2 arms) | (1 arm) | Monomer | % LMW |
| P2C1 | 0.4 | 98.7 | 0.9 | 0.0 | 6.2 | 1.3 | 92.4 | 0.00 |
| Initial | | | | | | | | |
| P1C1 | 0.4 | 98.7 | 0.9 | 0.0 | 5.6 | 1.4 | 93.0 | 0.00 |
| Initial | | | | | | | | |
| P1C2 | 0.5 | 98.6 | 0.9 | 0.0 | 4.3 | 1.4 | 94.2 | 0.00 |
| Initial | | | | | | | | |
| P1 | N/A | N/A | N/A | 0.2 | 6.4 | 1.3 | 92.1 | 0.00 |
| Initial | | | | | | | | |
| C1H | 0.6 | 98.5 | 1.0 | N/A | N/A | N/A | N/A | N/A |
| Initial | | | | | | | | |
| P2C1 | 0.9 | 99.1 | 0.0 | 0.0 | 8.6 | 4.6 | 86.8 | 0.00 |
| 12 M/5 C. | | | | | | | | |
| P1C1 | 0.8 | 99.2 | 0.0 | 0.0 | 9.0 | 4.7 | 86.3 | 0.00 |
| 12 M/5 C. | | | | | | | | |
| P1C2 | 0.9 | 99.1 | 0.0 | 0.0 | 9.6 | 4.8 | 85.6 | 0.00 |
| 12 M/5 C. | | | | | | | | |
| P1 | N/A | N/A | N/A | 0.5 | 8.4 | 4.5 | 86.6 | 0.01 |
| 12 M/5 C. | | | | | | | | |
| C1H | 0.9 | 98.5 | 0.6 | N/A | N/A | N/A | N/A | N/A |
| 12 M/5 C. | | | | | | | | |

Based on the twelve month data, the antibodies, when co-formulated, behaved well in solution, similar to the single antibody formulation. The co-formulation is shown to be stable at pH 5.0-6.0 with repulsive protein-protein interaction as measured by the protein diffusion interaction parameter kD, an indicator of colloidal and thermal stability.

### Example 4

### Additional Co-Formulations

This study evaluates the stability of an anti-CLTA4 antibody having the followings CDRs: HCDR1 of SEQ ID NO:35, HCDR2 of SEQ ID NO: 36, HCDR3 of SEQ ID NO: 37, LCDR1 of SEQ ID NO: 38, LCDR2 of SEQ ID NO: 39, and LCDR3 of SEQ ID NO: 40 on an IgG1 backbone co-formulated with pembrolizumab at various concentrations as follows:

TABLE 32

| | Pembrolizumab/ | | Anti- | Total |
|---|---|---|---|---|
| Co- | Anti-CTLA4 | | CTLA4 | Con- |
| formulations | ratio (w/w) | Pembro | antibody | centration |
| 25/200 | 8:1 | 23.5 mg/mL | 2.9 mg/mL | 26.4 mg/mL |
| 75/200 | 8:3 | 21.1 mg/mL | 7.9 mg/mL | 29.0 mg/mL |

The formulations were prepared as liquid formulations as follows:

TABLE 33

| | | | Cryoprotectant/ | | |
|---|---|---|---|---|---|
| | | | Tonicity | | Anti- |
| Formulation | Buffer | pH | modifier | Surfactant | oxidant |
| 25/200 A | L-Histidine | 5.5 | Sucrose (7%) | PS-80 | 0 |
| | (10 mM) | | | (0.02%) | |
| 25/200 B | L-Histidine | 5.5 | Sucrose (7%) | PS-80 | 1.6 mM |
| | (10 mM) | | | (0.02%) | |
| 25/200 C | L-Histidine | 5.5 | Sucrose (7%) | PS-80 | 10 mM |
| | (10 mM) | | | (0.02%) | |
| 75/200 A | L-Histidine | 5.5 | Sucrose (7%) | PS-80 | 0 |
| | (10 mM) | | | (0.02%) | |
| 75/200 B | L-Histidine | 5.5 | Sucrose (7%) | PS-80 | 1.6 mM |
| | (10 mM) | | | (0.02%) | |
| 75/200 C | L-Histidine | 5.5 | Sucrose (7%) | PS-80 | 10 mM |
| | (10 mM) | | | (0.02%) | |

The formulations were placed at three different storage conditions: 5° C. (ambient humidity), 25° C. (60% relative humidity), and 40° C. (75% relative humidity). The 75/200 formulations were also exposed to light stress (0 ICH, 0.5×ICH, or 1×ICH).

### Results

The results indicate that increasing methionine concentration lowers the subvisible particulates at all conditions, based on the measurement of MFI (data not shown). Increasing the methionine concentration lowered the % HMW species at 40° C. as observed by UP-SEC. Increasing the methionine concentration slightly lowers the turbidity at 40° C.

| Formulation 75/200 A | | Formulation 75/200 B | | Formulation 75/200 C | |
|---|---|---|---|---|---|
| 3 M, 5 C. | 3 M, 40 C. | 3 M 5 C. | 3 M 40 C. | 3 M 5 C. | 3 M 40 C. |

Purity by UPSEC %

| | | | | | | |
|---|---|---|---|---|---|---|
| High Molecular | 1.10 | 2.70 | 1.07 | 2.41 | 1.07 | 2.25 |
| Weight Species(%) | | | | | | |
| Monomer (%) | 98.8 | 96.7 | 98.9 | 97.0 | 98.9 | 97.1 |
| Low Molecular | 0.07 | 0.63 | 0.07 | 0.62 | 0.07 | 0.62 |
| Weight Species (%) | | | | | | |

Charge Variants by HP-IEX %

| | | | | | | |
|---|---|---|---|---|---|---|
| Acidic Variants anti- | 20.5 | 59.1 | 20.3 | 58.4 | 19.9 | 58.1 |
| PD1 | | | | | | |
| Total Main anti-PD1 | 56.4 | 29.3 | 56.6 | 29.6 | 56.6 | 29.6 |
| Basic Variants anti- | 23.1 | 11.6 | 23.1 | 12.0 | 23.5 | 12.3 |
| PD1 | | | | | | |
| Acidic Variants anti- | 14.8 | 60.5 | 14.9 | 59.2 | 14.9 | 59.1 |
| CTLA4 | | | | | | |
| Total Main anti- | 75.5 | 30.2 | 75.2 | 30.8 | 75.4 | 31.0 |
| CTLA4 | | | | | | |
| Basic Variants anti- | 9.8 | 9.3 | 9.9 | 10.0 | 9.7 | 9.9 |
| CTLA4 | | | | | | |
| pH | 5.4 | 5.4 | 5.4 | 5.4 | 5.4 | 5.4 |
| UV A350 | 0.065 | 0.105 | 0.067 | 0.097 | 0.065 | 0.090 |

### Example 5

### Long Term Stability of CTLA4 Mono-Formulation

The following liquid formulation was prepared using an anti-CTLA4 antibody having the following CDRs: 100 mg/mL anti-CTLA4 antibody, 10 mM L-Histidine buffer, 7% w/v Sucrose, 0.02% w/v Polysorbate 80, at pH5.5. The product was dispenses into 2R Type I Glass with an elastomeric stopper and aluminum seal. The fill volume was 2.0 mL, with an excess fill of 0.2 mL. Samples were placed on stability under the following conditions:
(i) 5° C./ambient Humidity
(ii) 25° C./60% Relative Humidity
(iii) 40° C./75% Relative Humidity
   Samples were tested initially, and at months 1, 3, 6, 9, and 12. The results are set forth in the following tables:

TABLE 34
5° C./ambient Humidity

| | Time Point (months) | | | | | |
|---|---|---|---|---|---|---|
| Attribute Measured | Initial | 1 | 3 | 6 ^{c} | 9 | 12 |
| Biological Potency | 95 | 96 | 94 | 106 | 97 | 101 |
| by Binding ELISA | | | | | | |

Purity by UPSEC %

| | | | | | | |
|---|---|---|---|---|---|---|
| High Molecular | 1.11 | 1.16 | 1.24 | 1.31 | 1.33 | 1.39 |
| Weight Species(%) | | | | | | |
| Monomer (%) | 98.9 | 98.8 | 98.7 | 98.7 | 98.6 | 98.6 |
| Low Molecular | <QL | <QL | <QL | <QL | <QL | <QL |
| Weight Species (%) | | | | | | |

Charge Variants by HP-IEX %

| | | | | | | |
|---|---|---|---|---|---|---|
| Acidic Variants | 15.42 | 15.46 | 15.00 | 15.71 | 15.79 | 15.96 |
| Total Main | 73.9 | 73.6 | 73.3 | 73.1 | 73.2 | 72.7 |
| Basic Variants | 10.73 | 10.95 | 11.65 | 11.17 | 11.02 | 11.30 |
| Purity by non- | 97.6 | 97.5 | 97.5 | 97.6 | 97.5 | 97.5 |
| reduced CE-SDS % | | | | | | |
| Purity by Reduced | 96.7 | 96.7 | 96.8 | 96.0 | 97.2 | 96.2 |
| CE-SDS % | | | | | | |
| pH | 5.6 | 5.6 | 5.6 | 5.6 | 5.6 | 5.6 |
| Protein | 52.3 | 52.7 | 53.2 | 53.3 | 52.0 | 52.3 |
| Concentration | | | | | | |
| UV A350 | 0.081 | 0.084 | 0.082 | 0.096 | 0.088 | 0.084 |

QL = Quantitation Limit (0.10%)
6 month samples pulled 2 weeks earlier than scheduled.

TABLE 35
25° C./60% Relative Humidity

| | Time Point (months) | | | | | |
|---|---|---|---|---|---|---|
| Attribute Measured | Initial | 1 | 3 | 6 ^{c} | 9 | 12 |
| Biological Potency | 95 | 92 | 92 | 95 | 99 | 96 |
| by Binding ELISA | | | | | | |

Purity by UPSEC %

| | | | | | | |
|---|---|---|---|---|---|---|
| High Molecular | 1.11 | 1.23 | 1.25 | 1.34 | 1.36 | 1.42 |
| Weight Species(%) | | | | | | |
| Monomer (%) | 98.9 | 98.7 | 98.5 | 98.3 | 98.0 | 97.5 |
| Low Molecular | <QL | <QL | 0.30 | 0.39 | 0.69 | 1.05 |
| Weight Species (%) | | | | | | |

Charge Variants by HP-IEX %

| | | | | | | |
|---|---|---|---|---|---|---|
| Acidic Variants | 15.42 | 16.92 | 20.74 | 26.70 | 33.41 | 38.97 |
| Total Main | 73.9 | 71.3 | 66.5 | 60.5 | 53.7 | 47.8 |
| Basic Variants | 10.73 | 11.81 | 12.78 | 12.83 | 12.87 | 13.20 |
| Purity by non- | 97.6 | 97.2 | 96.7 | 96.2 | 94.6 | 94.3 |
| reduced CE-SDS % | | | | | | |
| Purity by Reduced | 96.7 | 96.6 | 96.2 | 95.5 | 94.9 | 94.7 |
| CE-SDS % | | | | | | |
| pH | 5.6 | 5.6 | 5.6 | 5.6 | 5.6 | 5.6 |
| Protein | 52.3 | 52.8 | 53.4 | 52.8 | 52.3 | 51.9 |
| Concentration | | | | | | |
| UV A350 | 0.081 | 0.085 | 0.090 | 0.096 | 0.104 | 0.109 |

QL = Quantitation Limit (0.10%)
6 month samples pulled 2 weeks earlier than scheduled.

TABLE 36
40° C./75% Relative Humidity

| | Time Point (months) | | | | | |
|---|---|---|---|---|---|---|
| Attribute Measured | Initial | 1 | 3 | 6 ^{c} | 9 | 12 |
| Biological Potency | 95 | 89 | 100 | 91 | 95 | 89 |
| by Binding ELISA | | | | | | |

Purity by UPSEC %

| | | | | | | |
|---|---|---|---|---|---|---|
| High Molecular | 1.11 | 1.28 | 1.58 | 1.98 | 1.11 | 1.28 |
| Weight Species(%) | | | | | | |
| Monomer (%) | 98.9 | 98.1 | 95.7 | 93.9 | 98.9 | 98.1 |
| Low Molecular | <QL | 0.59 | 2.75 | 4.12 | <QL | 0.59 |
| Weight Species (%) | | | | | | |

Charge Variants by HP-IEX %

| | | | | | | |
|---|---|---|---|---|---|---|
| Acidic Variants | 15.42 | 33.50 | 60.13 | 79.20 | 15.42 | 33.50 |
| Total Main | 73.9 | 52.1 | 26.8 | 11.6 | 73.9 | 52.1 |
| Basic Variants | 10.73 | 14.38 | 13.08 | 9.16 | 10.73 | 14.38 |
| Purity by non- | 97.6 | 95.5 | 91.4 | 86.1 | 97.6 | 95.5 |
| reduced CE-SDS % | | | | | | |
| Purity by Reduced | 96.7 | 94.8 | 91.8 | 86.3 | 96.7 | 94.8 |
| CE-SDS % | | | | | | |
| pH | 5.6 | 5.6 | 5.6 | 5.6 | 5.6 | 5.6 |
| Protein | 52.3 | 52.9 | 53.4 | 53.0 | 52.3 | 52.9 |
| Concentration | | | | | | |
| UV A350 | 0.081 | 0.084 | 0.082 | 0.096 | 0.088 | 0.084 |

QL = Quantitation Limit (0.10%)
6 month samples pulled 2 weeks earlier than scheduled.

### Results:

As shown in the data above, no significant changes were observed when the formulation was stored at 5° C. It is expected that such formulation would be stable for a period of 24 months at 5° C. The biological potency of the formulation as determined by binding ELISA showed that under all conditions, no significant change in potency was observed. There was no change in pH as a function of storage time or condition.

Protein concentration of the samples under all storage conditions showed no significant change across all three conditions tested up to 12 months on stability.

Charge Variants (% acidic variants, % main and % basic variants) were determined by HP-IEX. For the 5° C. condition, no significant change was observed over the 12 months. For the 25° C. condition, there was an increase in % acidic variants, and a decrease in the % total main peak, over the 12 months, with the % basic variants remaining unchanged. These results are not unexpected considering the storage conditions. For the 40° C. condition, the % total main peak showed a significant decrease from the initial time point to the 6 month time point, the % acidic variants significantly increased with the % basic variants remained unchanged. These results are not unexpected considering the storage conditions.

Purity was determined by UP-SEC (% HMW species, % monomer, and % LMW species). There is no change in the % monomer or the % HMW species up to 12 months of stability at 5° C. No peaks were detected for LMW species for the 5° C. condition over the 12 months. For the 25° C. storage conditions, there was no change in HMW species, a slight decrease in % monomer and a slight increase in LMW species over the 12 months. At the 40° C. condition, there is a slight increase in % HMW species up to 6 months, and the % monomer decreased below 85% at the 6 month time point. These results are not unexpected given the storage condition.

There was no change in pH as a function of storage time or condition.

### Example 6

### Addition of Chelator as Optional Excipient

The stability of formulations in the presence or absence of a chelator (DTPA) was evaluated. To evaluate the stability of the formulations, the two formulations were filled into vials and staged on stability at 5° C. (ambient humidity), 25° C. (60% relative humidity), and 40° C. (75% relative humidity)—for twelve weeks protected from light. The two formulations are as follows:

Formulation

| Number | Description | | | | | |
|---|---|---|---|---|---|---|
| 1 | Anti- | 10 mM L- | 7% | 10 mM | 0.02% | NA |
| | CTLA4 | Histidine | Sucrose | L-Met | PS80 | |
| | antibody | buffer | (w/v) | | (w/v) | |
| | (50 mg/ml) | (pH = 5.5) | | | | |
| 2 | Anti- | 10 mM L- | 7% | 10 mM | 0.02% | 20 uM |
| | CTLA4 | Histidine | Sucrose | L-Met | PS80 | DTPA |
| | antibody | buffer | (w/v) | | (w/v) | |
| | (50 mg/ml) | (pH = 5.5) | | | | |

The colloidal stability of the samples were assessed by size exclusion chromatography (SEC) for purity in which the percentage of monomer was determined, as well as the percentages of high molecular weight species (HMW) and late eluting peaks (LMW species). The UPSEC data to evaluate the levels of High Molecular Weight Species (HMW or aggregates), % monomer and LMW (Low Molecular Weight species) is in the Table below.

TABLE 37

| | HMW % | LMW % | Main % |
|---|---|---|---|
| Formulation | Initial Value (T0) | Initial Value (T0) | Initial Value (T0) |
| 1 | 0.17 | 0.01 | 99.8 |
| 2 | 0.16 | 0.03 | 99.8 |

| | 2 W | 2 W | 2 W | 2 W | 2 W | 2 W | 2 W | 2 W | 2 W |
|---|---|---|---|---|---|---|---|---|---|
| Sample | 5° C. | 25° C. | 40° C. | 5° C. | 25° C. | 40° C. | 5° C. | 25° C. | 40° C. |
| 1 | ND | ND | 0.30 | ND | ND | 0.18 | ND | ND | 99.5 |
| 2 | ND | ND | 0.28 | ND | ND | 0.17 | ND | ND | 99.5 |
| | 4 W | 4 W | 4 W | 4 W | 4 W | 4 W | 4 W | 4 W | 4 W |
| Sample | 5° C. | 25° C. | 40° C. | 5° C. | 25° C. | 40° C. | 5° C. | 25° C. | 40° C. |
| 1 | 0.19 | 0.29 | 0.35 | 0.01 | 0.06 | 0.38 | 99.8 | 99.7 | 99.3 |
| 2 | 0.17 | 0.26 | 0.33 | 0.03 | 0.07 | 0.35 | 99.8 | 99.7 | 99.8 |
| | 8 W | 8 W | 8 W | 8 W | 8 W | 8 W | 8 W | 8 W | 8 W |
| Sample | 5° C. | 25° C. | 40° C. | 5° C. | 25° C. | 40° C. | 5° C. | 25° C. | 40° C. |
| 1 | 0.21 | 0.33 | 0.44 | 0.01 | 0.11 | 0.76 | 99.8 | 99.6 | 98.8 |
| 2 | 0.19 | 0.30 | 0.41 | 0.02 | 0.12 | 0.68 | 99.8 | 99.6 | 98.9 |
| | 12 W | 12 W | 12 W | 12 W | 12 W | 12 W | 12 W | 12 W | 12 W |
| Sample | 5° C. | 25° C. | 40° C. | 5° C. | 25° C. | 40° C. | 5° C. | 25° C. | 40° C. |
| 1 | 0.28 | 0.44 | 0.82 | 0.5 | 0.25 | 2.12 | 99.7 | 99.3 | 97.1 |
| 2 | 0.29 | 0.42 | 0.68 | 0.04 | 0.23 | 1.79 | 99.7 | 99.3 | 97.5 |

As shown in the table above, UP-SEC analysis of the samples to determine the percentage of HMW and percentage of monomer indicated that at 5° C., 25° C. and 40° C., both the formulations showed a trend of increase in % HMW peak and % LMW peak (and a consequent decrease in % monomer peak) for up to 12-week time point. At 25° C., both the formulations showed similar trends, but smaller changes, as compared to 40° C. At 5° C., no substantial changes were observed. Based on the data, Formulation 1 (no DTPA) showed a slight increase in % HMW and % LMW as compared to Formulation 2 (with DTPA). In addition, Formulation 1 showed a greater decrease of % monomer as compared to Formulation 2.

HP-IEX analysis of the samples to determine the chemical stability indicated that at 5° C., 25° C. and 40° C., both the formulations showed a trend of increase in % acidic peak and a consequent decrease in % monomer peak for up to 12-week time point. At 25° C., both the formulations showed similar trends, but smaller changes, as compared to 40° C. At 5° C., no substantial changes were observed (data not shown).

The results in the Table below show a trend of decreasing PS80 concentration with time up to the 8-week time point. At 25° C., both the formulations showed similar trends, but smaller changes, as compared to 40° C. At 5° C., no substantial changes were observed. Less degradation of PS80 was seen in Formulation 2 (anti-CTLA4 with DTPA) as compared to Formulation 1 (anti-CTLA4 antibody without DTPA) at 40° C.

| | Form. | T0 | 4 W | 8 W | 4 W | 8 W | 2 W | 4 W | 8 W |
|---|---|---|---|---|---|---|---|---|---|
| Polysorbate | 1 | 0.19 | 0.18 | 0.18 | 0.17 | 0.15 | 0.17 | 0.16 | 0.13 |
| 80 conc. (mg/ml) | 2 | 0.19 | 0.18 | 0.18 | 0.17 | 0.15 | 0.17 | 0.17 | 0.16 |

Thus, DTPA can also provide even greater stability to the formulations described herein.

What is claimed is:

1. A co-formulation comprising:
(i) about 1 mg/ml to about 25 mg/ml of an anti-CTLA4 antibody, wherein the anti-CTLA4 antibody comprises a heavy chain variable region comprising SEQ ID NO: 88, and a light chain variable region comprising SEQ ID NO: 48.
(ii) about 1 mg/ml to about 25 mg/ml of an anti-PD1 antibody, wherein the anti-PD1 antibody is pembrolizumab;
(iii) about 10 mM of L-histidine buffer;
(iv) about 7% weight/volume (w/v) sucrose;
(v) about 0.02% w/v polysorbate 80; and
(vi) about 10 mM L-methionine, and
wherein the co-formulation has a pH between 5.0 and 6.0,
wherein the anti-CTLA4 antibody and pembrolizumab are co-formulated.

2. The co-formulation of claim 1, wherein the ratio of the anti-PD1 antibody to the anti-CTLA4 antibody is 1:2, 1:1, 2:1, 10:1, 1:10, 8:3, or 8:1 w/w.

3. The co-formulation of claim 2, wherein the ratio of the anti-PD1 antibody to the anti-CTLA4 antibody is 8:1 w/w.

4. The co-formulation of claim 1, wherein the concentration of the anti-CTLA4 antibody is about 1.25 mg/ml, 2.5 mg/ml, 2.9 mg/ml, 5 mg/ml, 7.9 mg/ml, 10 mg/ml, 12.5 mg/ml, 25 mg/ml.

5. The co-formulation of claim 4, wherein the concentration of the anti-PD1 antibody or antigen binding fragment thereof is about 25 mg/mL, 22.7 mg/mL, 2.27 mg/mL, 21.1 mg/mL or 23.5 mg/ml.

6. The co-formulation of claim 1, comprising about 23.5 mg/mL of the anti-PD1 antibody and about 2.9 mg/mL anti-CTLA4 antibody.

7. The co-formulation of claim 1, further comprising a chelator, wherein the chelator is diethylenetriaminepentaacetic acid (DTPA).

8. The co-formulation of claim 1, wherein the co-formulation is contained in a glass vial or an injection device.

9. The co-formulation of claim 1, that is a liquid co-formulation, or that is frozen to at least below −70° C., or that is a reconstituted solution from a lyophilized co-formulation.

10. A method of treating cancer or chronic infection in a human patient in need thereof, the method comprising administering an effective amount of the co-formulation of claim 1.


SEQUENCE LISTING (100 sequences)

SEQ ID NO: 1 - protein, 15 aa, synthetic construct
  REGION 1..15: note = CDR1
RASKGVSTSGYSYLH

SEQ ID NO: 2 - protein, 7 aa, synthetic construct
  REGION 1..7: note = CDR2
LASYLES

SEQ ID NO: 3 - protein, 9 aa, synthetic construct
  REGION 1..9: note = CDR3
QHSRDLPLT

SEQ ID NO: 4 - protein, 111 aa, synthetic construct [sequence omitted]

SEQ ID NO: 5 - protein, 218 aa, synthetic construct
  REGION 1..218: note = Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
  REGION 1..5: note = CDR1
NYYMY

SEQ ID NO: 7 - protein, 17 aa, synthetic construct
  REGION 1..17: note = CDR2
GINPSNGGTNFNEKFKN

SEQ ID NO: 8 - protein, 11 aa, synthetic construct
  REGION 1..11: note = CDR3
RDYRFDMGFDY

SEQ ID NO: 9 - protein, 120 aa, synthetic construct [sequence omitted]

SEQ ID NO: 10 - protein, 447 aa, synthetic construct
  REGION 1..447: note = Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
  REGION 1..11: note = CDR1
RASQSVSSYLA

SEQ ID NO: 12 - protein, 7 aa, synthetic construct
  REGION 1..7: note = CDR2
DASNRAT

SEQ ID NO: 13 - protein, 9 aa, synthetic construct
  REGION 1..9: note = CDR3
QQSSNWPRT

SEQ ID NO: 14 - protein, 107 aa, synthetic construct [sequence omitted]

SEQ ID NO: 15 - protein, 214 aa, synthetic construct
  REGION 1..214: note = Light Chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = CDR1
NSGMH

SEQ ID NO: 17 - protein, 17 aa, synthetic construct
  REGION 1..17: note = CDR2
VIWYDGSKRYYADSVKG

SEQ ID NO: 18 - protein, 4 aa, synthetic construct
  REGION 1..4: note = CDR3
NDDY

SEQ ID NO: 19 - protein, 113 aa, synthetic construct [sequence omitted]

SEQ ID NO: 20 - protein, 440 aa, synthetic construct
  REGION 1..440: note = Heavy Chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, synthetic construct
  REGION 1..15: note = CDRL1
RASKSVSTSGFSYLH

SEQ ID NO: 22 - protein, 7 aa, synthetic construct
  REGION 1..7: note = CDRL2
LASNLES

SEQ ID NO: 23 - protein, 9 aa, synthetic construct
  REGION 1..9: note = CDRL3
QHSWELPLT

SEQ ID NO: 24 - protein, 5 aa, synthetic construct
  REGION 1..5: note = CDRH1
SYYLY

SEQ ID NO: 25 - protein, 17 aa, synthetic construct
  REGION 1..17: note = CDRH2
GVNPSNGGTNFSEKFKS

SEQ ID NO: 26 - protein, 11 aa, synthetic construct
  REGION 1..11: note = CDRH3
RDSNYDGGFDY

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 111 aa, synthetic construct
  REGION 1..111: note = Light Chain VR
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, synthetic construct
  REGION 1..111: note = Light Chain VR
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, synthetic construct; identical to SEQ ID NO: 10
  REGION 1..447: note = Heavy Chain

SEQ ID NO: 32 - protein, 218 aa, synthetic construct; identical to SEQ ID NO: 5
  REGION 1..218: note = Light Chain

SEQ ID NO: 33 - protein, 218 aa, synthetic construct
  REGION 1..218: note = Light Chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, synthetic construct
  REGION 1..218: note = Light Chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 8 aa, synthetic construct
  REGION 1..8: note = HCDR1
GFTFSDNW

SEQ ID NO: 36 - protein, 10 aa, synthetic construct
  REGION 1..10: note = HCDR2
IRNKPYNYET

SEQ ID NO: 37 - protein, 6 aa, synthetic construct
  REGION 1..6: note = HCDR3
TAQFAY

SEQ ID NO: 38 - protein, 6 aa, synthetic construct
  REGION 1..6: note = LCDR1
ENIYGG

SEQ ID NO: 39 - skipped

SEQ ID NO: 40 - protein, 9 aa, synthetic construct
  REGION 1..9: note = LCDR3
QNVLRSPFT

SEQ ID NO: 41 - protein, 9 aa, synthetic construct
  REGION 1..9: note = LCDR3
QNVLSRHPG

SEQ ID NO: 42 - protein, 9 aa, synthetic construct
  REGION 1..9: note = LCDR3
QNVLSSRPG

SEQ ID NO: 43 - protein, 115 aa, synthetic construct
  REGION 1..115: note = 8D2/8D2 (RE) VH
EVKLDETGGGLVQPGRPMKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 44 - protein, 106 aa, synthetic construct
  REGION 1..106: note = 8D2/8D2 (RE)
DIQMTQSPASLSASVGETVTITCGTSENIYGGLNWYQRKQGKSPQLLIFGATNLADGMSSRFSGSGSGRQYSLKISSLHPDDVATYYCQNVLRSPFTFGSGTKLEI

SEQ ID NO: 45 - protein, 115 aa, synthetic construct
  REGION 1..115: note = 8D2H1L1 VH
EVQLVESGGGLVQPGGSMRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 46 - protein, 107 aa, synthetic construct
  REGION 1..107: note = 8D2H1L1 VL
DIQMTQSPSSLSASVGDRVTITCRTSENIYGGLNWYQRKQGKSPKLLIYGATNLASGMSSRFSGSGSGTDYTLKISSLHPDDVATYYCQNVLRSPFTFGSGTKLEIK

SEQ ID NO: 47 - protein, 115 aa, synthetic construct
  REGION 1..115: note = 8D2H2L2 VH
EVQLVESGGGLVQPGGSMRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 48 - protein, 107 aa, synthetic construct
  REGION 1..107: note = 8D2H2L2 VH
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIK

SEQ ID NO: 49 - protein, 115 aa, synthetic construct
  REGION 1..115: note = 8D3H3L3 VH
EVQLVESGGGLVQPGGSLRLSCAASGFTFSDNWMNWVRQAPGKGLEWVAQIRNKPYNYETEYAASVKGRFTISRDDSKNSAYLQMNSLKTEDTAVYYCTAQFAYWGQGTLVTVSS

SEQ ID NO: 50 - protein, 107 aa, synthetic construct
  REGION 1..107: note = 8D3H3L3 VL
DIQMTQSPSSLSASVGDRVTITCRASENIYGGLNWYQQKPGKAPKLLIYGATSLASGVPSRFSGSGSGTDYTLTISSLQPEDFATYYCQNVLRSPFTFGSGTKLEIK

SEQ ID NO: 51 - protein, 115 aa, synthetic construct; identical to SEQ ID NO: 47
  REGION 1..115: note = 8D2H2L15 VH

SEQ ID NO: 52 - protein, 107 aa, synthetic construct
  REGION 1..107: note = 8D2H2L15 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSRHPGFGSGTKLEIK

SEQ ID NO: 53 - protein, 115 aa, synthetic construct; identical to SEQ ID NO: 47
  REGION 1..115: note = 8D2H2L17 VH

SEQ ID NO: 54 - protein, 107 aa, synthetic construct
  REGION 1..107: note = 8D2H2L17 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSSRPGFGSGTKLEIK

SEQ ID NO: 55 - protein, 115 aa, synthetic construct
  REGION 1..115: note = 4G10 murine
QVKLQESGPELVKPGASMKISCKASGYSFTGYTMNWVKQSHGKNLEWIGLINPYNNITNYNQKFMGKATFTVDKSSSTAYMELLRLTSEDSGVYFCARLDYRSYWGQGTLVTVSA

SEQ ID NO: 56 - protein, 126 aa, synthetic construct
  REGION 1..126: note = 4G10 murine
QAVVTQESALTTSPGETVTLTCRSSTGAVTTSNFANWVQEKPDHLFTSLIGGTNNRAPGVPARFSGSLIGDKAALTITGAQTEDEAIYFCALWYSNHWVFGGGTKLTVLGQPKSSPSVTLFQGQFC

SEQ ID NO: 57 - protein, 8 aa, synthetic construct
  REGION 1..8: note = HCDR1
GYSFTGYT

SEQ ID NO: 58 - protein, 5 aa, synthetic construct
  REGION 1..5: note = HCDR2
  SITE 4: note = MISC_FEATURE - X is M,V,L,I,G,A,S,T
GYTXN

SEQ ID NO: 59 - protein, 8 aa, synthetic construct
  REGION 1..8: note = HCDR
  SITE 6: note = MISC_FEATURE - X is N, D or E
  SITE 8: note = MISC_FEATURE - X is T, D, E, G or A
INPYNXIX

SEQ ID NO: 60 - protein, 17 aa, synthetic construct
  REGION 1..17: note = HCDR
  SITE 7: note = MISC_FEATURE - X is N, D
  SITE 9: note = MISC_FEATURE - X is T, D, E, G, or A
  SITE 12: note = MISC_FEATURE - X is A or N
  SITE 16: note = MISC_FEATURE - X is Q or M
LINPYNXIXNYXQKFXG

SEQ ID NO: 61 - protein, 6 aa, synthetic construct
  REGION 1..6: note = HCDR
LDYRSY

SEQ ID NO: 62 - protein, 8 aa, synthetic construct
  REGION 1..8: note = HCDR
ARLDYRSY

SEQ ID NO: 63 - protein, 9 aa, synthetic construct
  REGION 1..9: note = LCDR
TGAVTTSNF

SEQ ID NO: 64 - protein, 14 aa, synthetic construct
  REGION 1..14: note = LCDR
  SITE 13: note = MISC_FEATURE - X is P or A
GSSTGAVTTSNFXN

SEQ ID NO: 65 - protein, 7 aa, synthetic construct
  REGION 1..7: note = LCDR
  SITE 5: note = MISC_FEATURE - X is K, R or any amino acid except M or C
  SITE 7: note = MISC_FEATURE - X is S or P
GTNNXAX

SEQ ID NO: 66 - protein, 8 aa, synthetic construct
  REGION 1..8: note = LCDR
  SITE 3: note = MISC_FEATURE - X is W or any amino acid except M or C
  SITE 8: note = MISC_FEATURE - X is W or any amino acid except M or C
ALXYSNHX

SEQ ID NO: 67 - protein, 9 aa, synthetic construct
  REGION 1..9: note = LCDR
  SITE 3: note = MISC_FEATURE - X is W or any amino acid except M or C
  SITE 8: note = MISC_FEATURE - X is W or any amino acid except M or C
ALXYSNHXV

SEQ ID NO: 68 - protein, 115 aa, synthetic construct
  REGION 1..115: note = 4G10H1 humanized VH
QVQLVESGAELVKPGASMKISCKASGYSFTGYTMNWVKQAPGQGLEWIGLINPYNNITNYNQKFMGKATFTVDKSISTAYMELSRLTSDDSGVYFCARLDYRSYWGQGTLVTVSA

SEQ ID NO: 69 - protein, 115 aa, synthetic construct
  REGION 1..115: note = 4G10H3 humanized VH
QVQLVESGAEVKKPGASVKVSCKASGYSFTGYTMNWVRQAPGQGLEWIGLINPYNNITNYAQKFQGRVTFTVDTSISTAYMELSRLRSDDTGVYFCARLDYRSYWGQGTLVTVSA

SEQ ID NO: 70 - protein, 115 aa, synthetic construct
  REGION 1..115: note = 4G10H4 humanized VH
QVQLVESGAEVKKPGASVKVSCKASGYSFTGYTMNWVRQAPGQGLEWIGLINPYNDITNYAQKFQGRVTFTVDTSISTAYMELSRLRSDDTGVYFCARLDYRSYWGQGTLVTVSA

SEQ ID NO: 71 - protein, 115 aa, synthetic construct
  REGION 1..115: note = 4G10H5 humanized VH
QVQLVESGAEVKKPGASVKVSCKASGYSFTGYTMNWVRQAPGQGLEWIGLINPYNNIDNYAQKFQGRVTFTVDTSISTAYMELSRLRSDDTGVYFCARLDYRSYWGQGTLVTVSA

SEQ ID NO: 72 - protein, 115 aa, synthetic construct
  REGION 1..115: note = 4G10H consensus humanized VH
  SITE 11: note = MISC_FEATURE - X = V or L
  SITE 18: note = MISC_FEATURE - X = V or M
  SITE 20: note = MISC_FEATURE - X = V or I
  SITE 34: note = MISC_FEATURE - X = M, V, L, I, G, A, S, T
  SITE 38: note = MISC_FEATURE - X = R or K
  SITE 56: note = MISC_FEATURE - X = N or D or E
  SITE 58: note = MISC_FEATURE - X = T or D or E or G or A
  SITE 61: note = MISC_FEATURE - X = A or N
  SITE 65: note = MISC_FEATURE - X = Q or M
  SITE 67: note = MISC_FEATURE - X = R or K
  SITE 68: note = MISC_FEATURE - X = V or A
  SITE 74: note = MISC_FEATURE - X = T or K
  SITE 86: note = MISC_FEATURE - X = R or T
  SITE 87: note = misc_feature - Xaa can be any naturally occurring amino acid
  SITE 91: note = MISC_FEATURE - X =T or S
QVQLVESGAEXKKPGASXKXSCKASGYSFTGYTXNWVXQAPGQGLEWIGLINPYNXIXNYXQKFXGXXTFTVDXSISTAYMELSRLXSDDXGVYFCARLDYRSYWGQGTLVTVSA

SEQ ID NO: 73 - protein, 109 aa, synthetic construct
  REGION 1..109: note = 4G10L1 humanized VL
QAVVTQEPSLTVSPGGTVTLTCGSSTGAVTTSNFANWVQEKPGQAFRSLIGGTNNRASWVPARFSGSLLGGKAALTISGAQPEDEAEYFCALWYSNHWVFGGGTKLTVL

SEQ ID NO: 74 - protein, 109 aa, synthetic construct
  REGION 1..109: note = 4G10L3 humanized VL
QAVVTQEPSLTVSPGGTVTLTCGSSTGAVTTSNFPNWVQQKPGQAPRSLIGGTNNKASWTPARFSGSLLGGKAALTISGAQPEDEAEYYCALWYSNHWVFGGGTKLTVL

SEQ ID NO: 75 - protein, 109 aa, synthetic construct
  REGION 1..109: note = 4G10L consensus humanized VL
  SITE 35: note = misc_feature - Xaa can be any naturally occurring amino acid
  SITE 40: note = misc_feature - Xaa can be any naturally occurring amino acid
  SITE 46: note = misc_feature - Xaa can be any naturally occurring amino acid
  SITE 56: note = misc_feature - Xaa can be any naturally occurring amino acid
  SITE 58: note = misc_feature - Xaa can be any naturally occurring amino acid
  SITE 60: note = misc_feature - Xaa can be any naturally occurring amino acid
  SITE 89: note = misc_feature - Xaa can be any naturally occurring amino acid
  SITE 93: note = misc_feature - Xaa can be any naturally occurring amino acid
  SITE 98: note = misc_feature - Xaa can be any naturally occurring amino acid
QAVVTQEPSLTVSPGGTVTLTCGSSTGAVTTSNFXNWVQXKPGQAXRSLIGGTNNXAXWXPARFSGSLLGGKAALTISGAQPEDEAEYXCALXYSNHXVFGGGTKLTVL

SEQ ID NO: 76 - protein, 115 aa, synthetic construct; identical to SEQ ID NO: 68
  REGION 1..115: note = 4G10H1L1 VH

SEQ ID NO: 77 - protein, 109 aa, synthetic construct; identical to SEQ ID NO: 73
  REGION 1..109: note = 4G10H1L1 VL

SEQ ID NO: 78 - protein, 115 aa, synthetic construct; identical to SEQ ID NO: 69
  REGION 1..115: note = 4G10H3L3 VH

SEQ ID NO: 79 - protein, 109 aa, synthetic construct; identical to SEQ ID NO: 74
  REGION 1..109: note = 4G10H3L3 VL

SEQ ID NO: 80 - protein, 115 aa, synthetic construct; identical to SEQ ID NO: 70
  REGION 1..115: note = 4G10H4L3 VH

SEQ ID NO: 81 - protein, 109 aa, synthetic construct; identical to SEQ ID NO: 74
  REGION 1..109: note = 4G10H4L3 VL

SEQ ID NO: 82 - protein, 115 aa, synthetic construct; identical to SEQ ID NO: 71
  REGION 1..115: note = 4G10H5L3 VH

SEQ ID NO: 83 - protein, 109 aa, synthetic construct; identical to SEQ ID NO: 74
  REGION 1..109: note = 4G10H5L3 VL

SEQ ID NO: 84 - protein, 448 aa, synthetic construct
  REGION 1..448: note = Heavy Chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 215 aa, synthetic construct
  REGION 1..215: note = Light Chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 115 aa, synthetic construct
  REGION 1..115: note = 8D2/8D2 RE Variant 1 VH
EVKLDETGGGLVQPGRPIKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 87 - protein, 115 aa, synthetic construct
  REGION 1..115: note = 8D2H1L1 VARIANT 1 VH
EVQLVESGGGLVQPGGSIRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 88 - protein, 115 aa, synthetic construct
  REGION 1..115: note = 8D2H2L2 VARIANT 1 VH
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 89 - protein, 115 aa, synthetic construct
  REGION 1..115: note = 8D2H2L15 VARIANT 1
EVQLVESGGGLVQPGGSIRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 90 - protein, 115 aa, synthetic construct; identical to SEQ ID NO: 89
  REGION 1..115: note = 8D2H2L17 VARIANT 1 VH

SEQ ID NO: 91 - protein, 12 aa, synthetic construct
  REGION 1..12: note = CDRL1
RASQSVGSSYLA

SEQ ID NO: 92 - protein, 7 aa, synthetic construct
  REGION 1..7: note = CDRL2
GAFSRAT

SEQ ID NO: 93 - protein, 9 aa, synthetic construct
  REGION 1..9: note = CDRL3
QQYGSSPWT

SEQ ID NO: 94 - protein, 5 aa, synthetic construct
  REGION 1..5: note = CDRH1
SYTMH

SEQ ID NO: 95 - protein, 17 aa, synthetic construct
  REGION 1..17: note = CDRH2
FISYDGNNKYYADSVKG

SEQ ID NO: 96 - protein, 9 aa, synthetic construct
  REGION 1..9: note = CDRH3
TGWLGPFDY

SEQ ID NO: 97 - protein, 118 aa, synthetic construct
  REGION 1..118: note = HC variable
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 98 - protein, 108 aa, synthetic construct
  REGION 1..108: note = LC variable
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 99 - protein, 445 aa, synthetic construct
  REGION 1..445: note = Heavy chain
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 214 aa, synthetic construct
  REGION 1..214: note = Light Chain
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC